SEQUENCE LISTING (48 sequences)

SEQ ID NO: 1 - protein, 5 aa, Artificial Sequence
  CDR1-H1 Bax 8
IYTMD

SEQ ID NO: 2 - protein, 17 aa, Artificial Sequence
  CDR2-H1 Bax 8
YISPSGGNTSYADSVKG

SEQ ID NO: 3 - protein, 17 aa, Artificial Sequence
  CDR3-H1 Bax 8
RQYVLRYFDWSADAFDI

SEQ ID NO: 4 - protein, 11 aa, Artificial Sequence
  CDR1-L1 Bax 8
RASQSISSYLN

SEQ ID NO: 5 - protein, 7 aa, Artificial Sequence
  CDR2-L1 Bax 8
AASSLQS

SEQ ID NO: 6 - protein, 9 aa, Artificial Sequence
  CDR3-L1 Bax 8
QQSYSTPWT

SEQ ID NO: 7 - protein, 5 aa, Artificial Sequence
  CDR1-H1 Imalumab
IYSMN

SEQ ID NO: 8 - protein, 17 aa, Artificial Sequence
  CDR2-H1 Imalumab
SIGSSGGTTYYADSVKG

SEQ ID NO: 9 - protein, 9 aa, Artificial Sequence
  CDR3-H1 Imalumab
SQWLYGMDV

SEQ ID NO: 10 - protein, 11 aa, Artificial Sequence
  CDR1-L1 Imalumab
RSSQRIMTYLN

SEQ ID NO: 11 - protein, 7 aa, Artificial Sequence
  CDR2-L1 Imalumab
VASHSQS

SEQ ID NO: 12 - protein, 9 aa, Artificial Sequence
  CDR3-L1 Imalumab
QQSFWTPLT

SEQ ID NO: 13 - protein, 5 aa, Artificial Sequence
  CDR1-H1 Bax 74
KYYMI

SEQ ID NO: 14 - protein, 17 aa, Artificial Sequence
  CDR2-H1 Bax 74
WIGPSGGFTFYADSVKG

SEQ ID NO: 15 - protein, 12 aa, Artificial Sequence
  CDR3-H1 Bax 74
GTPDYGGNSLDH

SEQ ID NO: 16 - protein, 11 aa, Artificial Sequence
  CDR1-L1 Bax 74
RASQSIGTYLS

SEQ ID NO: 17 - protein, 7 aa, Artificial Sequence
  CDR2-L1 Bax 74
ATSRLQS

SEQ ID NO: 18 - protein, 9 aa, Artificial Sequence
  CDR3-L1 Bax 74
QQTYSTPLT

SEQ ID NO: 19 - protein, 5 aa, Artificial Sequence
  CDR1-H1 Bax 94
IYAMD

SEQ ID NO: 20 - protein, 17 aa, Artificial Sequence
  CDR2-H1 Bax 94
GIVPSGGFTKYADSVKG

SEQ ID NO: 21 - protein, 18 aa, Artificial Sequence
  CDR3-H1 Bax 94
VNVIAVAGTGYYYYGMDV

SEQ ID NO: 22 - protein, 12 aa, Artificial Sequence
  CDR1-L1 Bax 94, Bax A10
RASQGVSSSSLA

SEQ ID NO: 23 - protein, 7 aa, Artificial Sequence
  CDR2-L1 Bax 94
GTSSRAT

SEQ ID NO: 24 - protein, 8 aa, Artificial Sequence
  CDR3-L1 Bax 94
QQYGRSLT

SEQ ID NO: 25 - protein, 7 aa, Artificial Sequence
  CDR2-L1 Bax 152
GASNRAT

SEQ ID NO: 26 - protein, 5 aa, Artificial Sequence
  CDR1-H1 BaxA10
WYAMD

SEQ ID NO: 27 - protein, 17 aa, Artificial Sequence
  CDR2-H1 Bax A10
GIYPSGGRTKYADSVKG

SEQ ID NO: 28 - protein, 12 aa, Artificial Sequence
  CDR1-L1 Bax A10, Bax94
RASQGVSSSSLA

SEQ ID NO: 29 - protein, 118 aa, Artificial Sequence
  Sequences VH (wt)
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSS

SEQ ID NO: 30 - protein, 118 aa, Artificial Sequence
  VH L5Q
EVQLQESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSS

SEQ ID NO: 31 - protein, 118 aa, Artificial Sequence
  VH L5Q W97Y
EVQLQESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQYLYGMDVWGQGTTVTVSS

SEQ ID NO: 32 - protein, 107 aa, Artificial Sequence
  Sequences VL (wt)
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIK

SEQ ID NO: 33 - protein, 107 aa, Artificial Sequence
  VL F49Q A51G
DIQMTQSPSSLSASVGDRVTITCRSSQRIMTYLNWYQQKPGKAPKLLIYVGSHSQSGVPSRFRGSGSETDFTLTISGLQPEDSATYYCQQSFWTPLTFGGGTKVEIK

SEQ ID NO: 34 - protein, 107 aa, Artificial Sequence
  VL F49Y/A51G/W93F
DIQMTQSPSSLSASVGDRVTITCRSSQRIMTYLNWYQQKPGKAPKLLIYVGSHSQSGVPSRFRGSGSETDFTLTISGLQPEDSATYYCQQSFFTPLTFGGGTKVEIK

SEQ ID NO: 35 - protein, 107 aa, Artificial Sequence
  VL M30L/F49Y/A51G/W93F
DIQMTQSPSSLSASVGDRVTITCRSSQRILTYLNWYQQKPGKAPKLLIYVGSHSQSGVPSRFRGSGSETDFTLTISGLQPEDSATYYCQQSFFTPLTFGGGTKVEIK

SEQ ID NO: 36 - protein, 107 aa, Artificial Sequence
  VL M30L/F49Y/A51G/P80S/W93F
DIQMTQSPSSLSASVGDRVTITCRSSQRILTYLNWYQQKPGKAPKLLIYVGSHSQSGVPSRFRGSGSETDFTLTISGLQSEDSATYYCQQSFFTPLTFGGGTKVEIK

SEQ ID NO: 37 - protein, 329 aa, Artificial Sequence
  CH1-CH3 wt
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 329 aa, Artificial Sequence
  CH1-CH3, L234A/L235A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 329 aa, Artificial Sequence
  CH1-CH3, L234A/L235A/I253A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 329 aa, Artificial Sequence
  CH1-CH3, L234A/L235A/H310A/H435Q
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 41 - protein, 107 aa, Artificial Sequence [sequence omitted]

SEQ ID NO: 42 - protein, 214 aa, Artificial Sequence
  C0090 LC
DIQMTQSPSSLSASVGDRVTITCRSSQRILTYLNWYQQKPGKAPKLLIYVGSHSQSGVPSRFRGSGSETDFTLTISGLQSEDSATYYCQQSFFTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 447 aa, Artificial Sequence
  C0090 HC
EVQLQESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQYLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 4 aa, Artificial Sequence
  MIF catalytic motif
CALC

SEQ ID NO: 45 - protein, 7 aa, Artificial Sequence
  oxMIF epitope
EPCALCS

SEQ ID NO: 46 - protein, 214 aa, Artificial Sequence
  C0083/C0115 LC
DIQMTQSPSSLSASVGDRVTITCRSSQRIMTYLNWYQQKPGKAPKLLIYVGSHSQSGVPSRFRGSGSETDFTLTISGLQPEDSATYYCQQSFFTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 447 aa, Artificial Sequence
  C0115/C0118 HC
EVQLQESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQYLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - DNA, 23 bp, Artificial Sequence
  MIF gene
ttggtgtttacgatgaacatcgg

(19) United States
(12) Patent Application Publication
(10) Pub. No.: US 2024/0307562 A1
(43) Pub. Date: Sep. 19, 2024

(54) ANTI-oxMIF RADIOIMMUNOCONJUGATE

(71) Applicant: OncoOne Research & Development GmbH, Klosterneuburg (AT)

(72) Inventors: Alexander SCHINAGL, Vienna (AT); Randolf KERSCHBAUMER, Klosterneuburg (AT); Robert Michael THIELE, Seyring (AT)

(21) Appl. No.: 18/275,179

(22) PCT Filed: Feb. 2, 2022
(86) PCT No.: PCT/EP2022/052463

§ 371 Date: Jul. 31, 2023

(30) Foreign Application Priority Data
Feb. 3, 2021 (EP) 21155064.5

(51) Int. Cl. A61K 51/10; A61K 9/00

(52) U.S. Cl. CPC A61K 51/1021; A61K 9/0019; A61K 51/1096; A61K 2121/00; A61K 2123/00

(57) ABSTRACT

The present invention relates to the development of methods and tools effective for treating, preventing, and diagnosing cancer. Specifically, the present invention is directed to a radioisotope conjugated to an anti-oxMIF antibody (anti-oxMIF radioim-munoconjugate) with improved properties such as reduced aggregation potential and reduced hydrophobicity due to selected amino acid substitutions in the light and heavy chain variable domains and methods of treating, preventing, and diagnosing cancer comprising using the anti-oxMIF radioimmunoconjugate.

### FIELD OF THE INVENTION

The present invention relates to the development of methods and tools effective for treating, preventing, and diagnosing cancer. Specifically, the present invention is directed to a radioisotope conjugated to an anti-oxMIF antibody (anti-oxMIF radioimmunoconjugate) with improved properties such as reduced aggregation potential and reduced hydrophobicity due to selected amino acid substitutions in the light and heavy chain variable domains and methods of treating, preventing, and diagnosing cancer comprising using the anti-oxMIF radioimmunoconjugate.

### BACKGROUND OF THE INVENTION

The treatment of cancer by radio-immunotherapy (RIT) involves injecting the patient with a radioactive isotope ‘bullet’ connected to a specific cancer cell vector such as a monoclonal antibody, with the aim of selectively destroying targeted tumor cells. During radioactive decay, photons, electrons or even heavier particles are emitted and damage or kill cells along their trajectory. Radio-immunotherapy (RIT) is still a relatively new modality for cancer therapy, which started using beta-emitting radionuclides. During the last decade the RIT armamentarium of radioisotopes has been enriched by the addition of commercially available Lutetium-177 (177Lu), an intermediate energy beta emitter (beta max 0.13 MeV) with 0.7 mm range in tissue and a long physical half-life of 6.7 days. 177Lu demonstrated encouraging results in therapeutic clinical trials especially of somatostatin receptors-binding radiolabeled peptides. Due to its radiolanthanide chemistry, 177Lu is a residualizing radioisotope which is excreted primarily via hepatobiliary route (Phaeton R. et al., 2016). Recent experiments have also proved alpha emitters to be effective in destroying tumor cells. They are considered to be especially attractive for the treatment of blood-borne cancers and micro-metastatic tumors (where cancer cells are typically present throughout the body). Another likely area of use for alpha-immunotherapy is in treating the small numbers of cancer cells that may remain after high-dose chemotherapy or surgery. However, radio-immunotherapy applications predominantly relate to hematologic malignancies. Therapeutic efficacy of solid tumor RIT is often limited by the inadequate penetration of radioimmunoconjugate (RIC) as insufficient tumor penetration is a key factor limiting the efficacy of RIT (Huang C-Y, et al., 2012).

Antibodies are used as cancer vectors for radio-imaging of selected tumors. This involves injecting the patient with a radioactive isotope as ‘diagnostic probe’ connected to an antibody specific for a tumor. After injection the patient undergoes non-invasive neuroimaging techniques, such as SPECT or PET, which allows localization of the tumor and monitoring of disease progression. ⁸⁹Zr is considered as an optimal nuclide for PET due to its half-life, physicochemical characteristics for protein conjugation and availability (Warram J. M. et al., 2014; Carter L. M. et al., 2018).

RIT or radio-imaging is performed by administering to cancer patients so-called radioimmunoconjugates, which are constructs comprising a radionuclide with desirable properties linked to an antibody. Generally, the linking of the radionuclide to the antibody is done by means of a chelating agent. In the body, the antibody will carry the radionuclide to a diseased tissue expressing a corresponding antigen. RIT and radio-imaging require a tumor specific target with specific expression in tumor tissue, but no or minimal expression in normal tissue.

The cytokine Macrophage Migration Inhibitory Factor (MIF) has been described as early as 1966 (David, J. R., 1966; Bloom B. R. and Bennet, B., 1966). MIF, however, is markedly different from other cytokines and chemokines, because it is constitutively expressed, stored in the cytoplasm and present in the circulation of healthy subjects. Due to the ubiquitous nature of this protein, MIF can be considered as an inappropriate target for therapeutic intervention. However, MIF occurs in two immunologically distinct conformational isoforms, termed reduced MIF (redMIF) and oxidized MIF (oxMIF) (Thiele M. et al., 2015). RedMIF was found to be the abundantly expressed isoform of MIF which is abundantly expressed in healthy and diseased subjects and might reflect a latent zymogenic form of MIF (Schinagl. A. et al., 2018). In contrast, oxMIF seems to be a disease related isoform which can be detected in tumor tissue, specifically in tumor tissue from patients with colorectal, pancreatic, ovarian and lung cancer outlining a high tumor specificity of oxMIF (Schinagl. A. et al., 2016). Although oxMIF seems to fulfill the requirements as tumor specific target, several facts did not make oxMIF an obvious target. OxMIF is not precisely characterized, the molecular mechanism of disease related generation of oxMIF is still unclear and oxMIF is not considered a cell surface receptor.

Antibodies targeting oxMIF showed efficacy in in vitro and in vivo models of cancer (Hussain F. et al., 2013; Schinagl. A. et al., 2016). An oxMIF specific antibody (Imalumab) demonstrated an acceptable safety profile, satisfactory tissue penetration and indications for anti-tumor activity in a phase 1 clinical trial. However, a phase IIa combination study investigating Imalumab plus 5-fluorouracil/leucovorin or panitumumab versus standard of care in patients with mCRC, was terminated prematurely based on an overall benefit-risk assessment by the data safety monitoring board. (Mahalingam D. et al., 2015; Mahalingam D. et al., 2020).

Thiele M. et al., (2015) reports accumulation of a radiolabeled anti-oxMIF antibody in a murine pancreatic tumor model.

WO2019/234241A1 discloses anti-oxMIF/anti-CD3 bispecific antibodies which can be labeled with a radioisotope.

WO2009/086920A1 describes the anti-oxMIF antibody Bax69 (Imalumab).

Protein aggregation, specifically antibody aggregation is frequently observed at several stages of bioprocessing, including protein expression, purification and storage. Antibody aggregation can affect the overall yield of therapeutic protein manufacturing processes and may contribute to stability and immunogenicity of therapeutic antibodies. Protein aggregation of antibodies thus continues to be a significant problem in their developability and remains a major area of focus in antibody production. Antibody aggregation can be triggered by hydrophobic patches and partial unfolding of its domains, leading to monomer-monomer association followed by nucleation and aggregate growth. Although the aggregation propensities of antibodies and antibody-based proteins can be affected by the external experimental conditions, they are strongly dependent on the intrinsic antibody properties as determined by their sequences and structures.

The aggregation-resistance or aggregation-propensity of antibodies and proteins comprising antigen binding domains thereof is usually limited by the most aggregation prone domain(s) contained therein and by the strength of its interaction with surrounding domains (if present). Constant domains of antibodies generally do not aggregate and do not vary considerably. Accordingly, the weakest domains of an antibody regarding aggregation potential and stability are generally considered to be the variable domains (e.g., heavy chain variable domain (V_{H}) and/or light chain variable domain (V_{L}), Ewert S. et al., 2003). In this regard, incorporation of aggregation-prone V_{H} or V_{L} domains into otherwise stable recombinant antibody products often imparts these generally undesirable traits to the new recombinant design. Thus, engineering a variable domain to be aggregation-resistant is most likely rendering the entire protein, which comprises that variable domain, aggregation-resistant. Various strategies have been proposed for reducing aggregation of variable domains, e.g., rational design of aggregation-resistant proteins, complementarity determining region (CDR) grafting, introducing disulfide bonds into a variable domain or removing surface exposed hydrophobic patches. However, these methods show disadvantages, as they often alter binding properties such as affinity or specificity, are not generally applicable as they require specialized knowledge, are often laborious and might introduce immunogenic epitopes.

Further intrinsic properties of proteins such as hydrophobicity also play important roles in antibody solubility. Low solubility of these therapeutic proteins due to surface hydrophobicity may render formulation development more difficult and may lead to poor bio-distribution, undesirable pharmacokinetics behavior and immunogenicity in vivo. Decreasing the overall surface hydrophobicity of candidate monoclonal antibodies could thus provide benefits and cost savings relating to purification and dosing regimens.

Although increasing efforts are made in RIT and radio-imaging and despite the growing number of monoclonal antibodies used in clinical trials, there is a continuous need for improved radioimmunotherapy and imaging providing efficient therapeutic effects in cancer treatment and diagnosis, specifically involving antibodies with highly selective binding to tumor associated antigens.

### SUMMARY OF THE INVENTION

It is the objective of the present invention to provide a radioimmunoconjugate with highly selective tumor-binding properties.

The objective is solved by the subject matter of the present invention. Although oxMIF seems to fulfill the requirements as tumor specific target, several facts did not make oxMIF an obvious target for radioimmunotherapy. OxMIF is not precisely characterized, the molecular mechanism of disease related generation of oxMIF is still unclear and oxMIF is not considered a cell surface receptor.

In spite of this, it has been surprisingly shown that a radiolabeled anti-oxMIF antibody or antigen binding fragment thereof (anti-oxMIF radioimmunoconjugate) is highly selective due to its oxMIF-specificity and is highly cytotoxic due to the presence of a radioisotope, when used in radio-immunotherapy.

The radioimmunoconjugate can also be readily detected in tumor tissue due to its oxMIF-specificity and the appropriate radionuclide, when used in radio-imaging.

The radioimmunoconjugate is also highly selective due to its oxMIF-specificity and allows diagnosis and therapy due to the radionuclide, when used in a theranostic approach. The combination of a radionuclide and an anti-oxMIF antibody has surprisingly shown that the radiolabeled anti-oxMIF antibody has a relevant biodistribution, tumor uptake and efficacy, already proven in a mouse model. Therefore, the inventive radioimmunoconjugate is highly efficient for cancer therapy and also highly valuable for tumor detection and diagnosis.

The present invention discloses a radioimmunoconjugate (RIC) comprising a recombinant anti-oxMIF antibody or an antigen binding fragment thereof.

The present invention specifically provides a radioimmunoconjugate (RIC) comprising a recombinant anti-oxMIF antibody or an antigen binding fragment thereof, comprising:
(a1) a light chain variable domain comprising SEQ ID NO: 32 having at least one of amino acid substitutions M30L, F49Y, A51G, P80S, W93F, or
(a2) a light chain variable domain comprising SEQ ID NO: 32 with 1, 2, 3, 4, or 5 amino acid substitutions further comprising
   a conserved tyrosine at position 36, and
   at least one of amino acid substitutions M30L, F49Y, A51G, P80S, W93F; and
(b1) a heavy chain variable domain comprising SEQ ID NO: 29; or
(b2) a heavy chain variable domain comprising SEQ ID NO: 29 and the amino acid substitutions L5Q and/or W97Y, or
(b3) a heavy chain variable domain comprising SEQ ID NO: 29 having at least one of amino acid substitutions L5Q or W97Y, and 1, 2, 3, 4 or 5 further amino acid substitutions;
wherein amino acid positions are numbered according to Kabat,
wherein said antibody or antigen binding fragment thereof has reduced aggregation potential and reduced hydrophobicity compared to an antibody or an antigen binding fragment thereof comprising SEQ ID NO: 32 and SEQ ID NO: 29 lacking the amino acid substitutions.

The related germline V_{L} sequences of the anti-oxMIF antibody can have a high variability at positions D1, M4, S10, and L11 of framework 1.

According to a specific embodiment, the anti-oxMIF antibody therefore can further comprise a light chain variable domain comprising SEQ ID NO: 32 with 1, 2, 3, 4, or 5 amino acid substitutions in addition to further substitutions at positions D1, M4, S10, and L11.

The related germline VH sequences can have a high variability at the following positions: E1, S74 and T77.

According to a further specific embodiment, the anti-oxMIF antibody therefore can further comprise a heavy chain variable domain comprising SEQ ID NO: 29 with 1, 2, 3, 4, or 5 amino acid substitutions in addition to further substitutions at positions E1, S74 and T77.

In an alternative embodiment of the invention, herein provided is a radioimmunoconjugate (RIC) comprising a recombinant anti-oxMIF antibody or an antigen binding fragment thereof, comprising:
(a) an anti-oxMIF antibody light chain variable domain, comprising a light chain variable domain with at least 95% sequence identity to SEQ ID NO: 32, comprising a tyrosine at position 36 and further comprising at least one of amino acid substitutions M30L, F49Y, A51G, P80S, W93F, and
(b) a heavy chain variable domain comprising SEQ ID NO: 29 and amino acid substitution W97Y, or a heavy chain variable domain comprising at least 95% sequence identity to SEQ ID NO: 29 and further comprising amino acid substitution W97Y.

According to yet a further embodiment, the radioimmunoconjugate contains a light chain variable domain with the amino acid substitution W93F and a heavy chain variable region comprising the amino acid substitution W97Y.

According to a specific embodiment of the invention, the anti-oxMIF antibody or an antigen binding fragment thereof is directly labelled or labelled using acyclic, monocyclic, macrocyclic bifunctional chelating agents, in particular a chelating agent selected from p-SCN-bn-DOTA, p-NH2-Bn-DOTA, DOTA-NHS and p-SCN-Bn-deferoxamine comprising a chelating group and a functional/reactive linker.

According to a specific embodiment, the radioimmunoconjugate comprises a chelating group, specifically a chelating group resulting from coupling the anti-oxMIF antibody or an antigen binding fragment thereof to a chelating agent, in particular a chelating group selected from DOTA, a hexadentate chelator such as deferoxamine B (CAS No. 70-51-9), and an octadentate chelator such as the DFO derivative DFO* or an oxygen containing analogue thereof, such as oxoDFO*.

Specifically, the DFO can be of following structure:

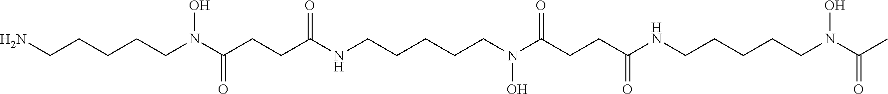 [SMILES: CC(=O)N(O)CCCCCNC(=O)CCC(=O)N(O)CCCCCNC(=O)CCC(=O)N(O)CCCCCN]

Specifically, DFO* can be of following structure:

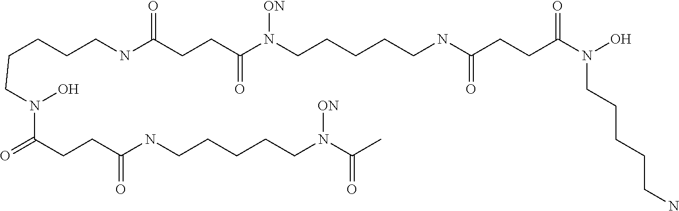

According to the invention, any alpha- or beta-emitting radioisotope useful in therapeutic application can be linked to the anti-oxMIF antibody, specifically it can be ¹¹C, ¹³N, ¹⁵O, ¹⁸F, ³²P, ⁶⁴Cu, ⁶⁷Cu, ⁶⁷Ga, ⁸⁹Zr, ⁹⁰Y, ⁹⁹mTc, ¹⁰³Pd, ¹⁰⁵Rh, ¹⁰⁹Pd, ¹¹¹Ag, ¹¹¹In, ¹²³I, ¹²⁴I, ¹²⁵I, ¹³¹I, ¹⁴⁰La, ¹⁴⁹Tb, ¹⁴⁹Pm, ¹⁵³Sm, ¹⁵⁹Gd, ¹⁶⁵Dy, ¹⁶⁶Dy, ¹⁶⁶Ho, ¹⁶⁹Yb, ¹⁷⁵Yb, ¹⁷⁷Lu, ²²⁷Th, ¹⁸⁶Re, ¹⁸⁸Re, ¹⁹²Ir, ¹⁹³mPt, ¹⁹⁵mPt, ¹⁹⁸Au, ¹⁹⁹Au, ²¹¹At, ²¹²Pb, ²¹²Bi, ²¹³Bi, ²²⁵Ac, ²²³Ra, ²²⁷Th, specifically the radioisotope is ⁶⁷Ga, ⁸⁹Zr, ¹¹¹In, ¹²⁴I, ¹³¹I, ¹⁷⁷Lu, ²²⁵Ac.

In a specific embodiment there is provided a radioimmunoconjugate as described herein, comprising a Fc variant domain of a wild-type human IgG1 constant domain having SEQ ID NO: 37, comprising at least one amino acid substitution at any one of positions L234, L235, G236, G237, N297, L328, or P329, and wherein said radioimmunoconjugate exhibits decreased binding to Fcγ-receptors compared to a radioimmunoconjugate comprising the wildtype IgG1 Fc region.

In a further specific embodiment, the radioimmunoconjugate comprises an Fc variant domain of a wild-type human IgG1 constant domain of SEQ ID NO: 37, comprising one, two or three amino acid substitutions at any one of positions, I253, H310, H435, and wherein said radioimmunoconjugate exhibits reduced affinity to the human FcRn compared to a radioimmunoconjugate comprising the wildtype IgG1 Fc region.

Specifically, the radioimmunoconjugate comprises SEQ ID NO: 38 and exhibits reduced affinity to the human FcRn compared to a radioimmunoconjugate comprising the wildtype IgG1 Fc region.

In a specific embodiment, the radioimmunoconjugate comprises an amino acid sequence selected from the group consisting of SEQ ID NOs: 31, 33, 34, 35, and 36.

In a further specific embodiment, the radioimmunoconjugate comprises SEQ ID NOs: 29 and 34; SEQ ID NOs: 30 and 33, SEQ ID NOs: 30 and 34, SEQ ID NOs: 31 and 34, SEQ ID NOs: 31 and 36, or SEQ ID NOs: 31 and 33, in combination with any one of SEQ ID NOs: 37, 38, 39, and 40.

More specifically, the radioimmunoconjugate described herein can be, but is not limited to an IgG, IgG-fusion protein, Fv, scFv, scFv-fusion protein, diabody, diabody fusion protein, Fab, Fab-fusion protein, scFab, scFab-fusion protein, Fab′, and F(ab′)2, Fab′-SH, Fcab, Fcab fusion protein, a fusion protein of two or more single chain antibodies, minibody, and small immune protein (SIP) format.

According to an embodiment, the radioimmunoconjugate described herein is for use in the preparation of a medicament.

In a specific embodiment, a pharmaceutical composition is provided herein, comprising the radioimmunoconjugate described herein, optionally together with a pharmaceutical carrier or adjuvant.

Specifically, the pharmaceutical composition is formulated for intravenous administration.

According to a further embodiment, the pharmaceutical composition is for use in the treatment of a subject suffering from cancer, specifically in the treatment of tumors, specifically of hematological or solid tumors, more specifically for the treatment of colorectal cancer, ovarian cancer, breast cancer, brain tumors, prostate cancer, pancreas cancer, and lung cancer.

An aspect of the present invention relates to the use of an effective amount of the pharmaceutical composition of the present invention in a method for treatment of any one of the diseases or symptoms listed herein.

An embodiment of the present invention relates to the use of the radioimmunoconjugate of the present invention in combination with or in addition to other therapy.

In an embodiment of the present invention the other therapy is selected from chemotherapy, monoclonal antibody therapy, surgery, radiotherapy, and/or photodynamic therapy.

According to an embodiment, the radioimmunoconjugate described herein is used for the detection or determination of the oxMIF level in the cancer cells of a subject, specifically detection is performed ex vivo in a sample of the subject. Potential patient groups may then be divided into subgroups (strata, blocks) by stratification according to the oxMIF level, wherein each strata represents a particular section of the patient population.

Specifically, the radioimmunoconjugate is used for cancer diagnosis in a subject.

In an embodiment herein provided is a method for in vitro diagnosing cancer, wherein the radioimmunoconjugate is used for in vitro detecting tumor cells in a sample of a subject or for in vivo detecting tumor cells or tumors in a subject. Said sample can be for example a blood sample, a tissue sample, e.g. from biopsy.

Further provided herein is a method for diagnosing cancer, wherein the radioimmunoconjugate is used for detecting tumor cells in a subject or, in vitro, in a sample of the subject.

A further embodiment relates to a diagnostic kit comprising the radioimmunoconjugate described herein.

In an alternative embodiment, a kit for production of the radioimmunoconjugate is provided, comprising two or more vials, wherein one vial contains a conjugate comprising the chelator linked to an anti-ox-MIF antibody and a second vial contains a radioisotope, specifically selected from ⁶⁷Ga, ⁸⁹Zr, ¹¹¹In, ¹²⁴I, ¹³¹I, ¹⁷⁷Lu, ²²⁵Ac. Specifically, the content of one or several of the vials is lyophilized or in solution. More specifically, the kit further comprises a manual containing instructions to prepare the radioimmunoconjugate described herein.

Further provided herein is a method for treating cancer using the radioimmunoconjugate described herein or a pharmaceutical composition comprising said radioimmunoconjugate.

Further provided is an isolated nucleic acid encoding the recombinant anti-oxMIF antibody or antigen binding fragment thereof of the radioimmunoconjugate described herein.

In a further embodiment, an expression vector comprising the nucleic acid molecule(s) encoding the recombinant anti-oxMIF antibody or antigen binding fragment thereof of the radioimmunoconjugate.

In a further embodiment there is provided a host cell containing the nucleic acid of the oxMIF antibody described herein or an expression vector comprising the nucleic acid molecule(s) described herein.

In yet a further embodiment, a method for producing the radioimmunoconjugate is provided, comprising expressing a nucleic acid encoding an oxMIF antibody or antigen binding fragment in a host cell and further labeling said antibody or antigen binding fragment with a radioisotope.

### FIGURES

FIG. 1: Purity and integrity analysis of DOTA-C0008; A: DOTA-C0008 SE-HPLC profile (λ=280 nm). B: Coomassie stained SDS PAGE: 1: molecular weight standard; 2: C0008 non-reduced; 3: C0008 reduced; 4: Concentrated C0008 (labeling batch) non-reduced; 5: Concentrated C0008 (labeling batch) reduced; 6: DOTA-C0008 non-reduced; 7: DOTA-C0008 reduced.

FIG. 2: Determination of the number of DOTA molecules grafted to C0008 determined by ITLC: ITLC separation of DOTA-C0008 which was incubated with a mixture of tracer amounts of radioactive indium (¹¹¹In) and stable indium (¹¹⁵In) at 2 (lane 6), 4 (lane 7), 6 (lane 8) or 8 (lane 9) fold excess of ^{111/115} In to the DOTA conjugated antibody.

FIG. 3: Analysis of radiolabeled C0008 by ITLC and Autoradiography: A: ITLC of radiolabeled C0008 (Lane 3: ¹⁷⁷Lu labelling reaction with DOTA-C0008; Lane 4: ¹⁷⁷Lu labelling reaction with C0008); B: Autoradiogram of ITLC film lane 3 (¹⁷⁷Lu-DOTA-C0008 after DTPA addition; Peak 1 reflects free ¹⁷⁷Lu-DTPA and Peak 2 reflects ¹⁷⁷Lu-DOTA-C0008).

FIG. 4: Plasma stability of ¹⁷⁷Lu-DOTA-C0008 (autoradiography): Autoradiogram of ¹⁷⁷Lu-DOTA-C0008 incubated in mouse plasma for 7 days; 1: ¹⁷⁷Lu-DOTA-C0008 derivatives and free ¹⁷⁷Lu; 2: ¹⁷⁷Lu-C0008.

FIG. 5: Plasma stability of ¹⁷⁷Lu-DOTA-C0008 (SDS-PAGE): Reduced SDS PAGE with ¹⁷⁷Lu-DOTA-C0008 in mouse plasma (left: Coomassie staining; right: Autoradiography). Lane 1 and Lane 10: molecular weight standards; Lane 2: DOTA-C0008; Lane 3: ¹⁷⁷Lu-DOTA-C0008 in buffer (TO); Lane 5: ¹⁷⁷Lu-DOTA-C0008 (T0 hours in plasma); Lane 6: ¹⁷⁷Lu-DOTA-C0008 (1 day in plasma), Lane 7: ¹⁷⁷Lu-DOTA-C0008 (2 days in plasma); Lane 8: ¹⁷⁷Lu-DOTA-C0008 (3 days in plasma); Lane 9: ¹⁷⁷Lu-DOTA-C0008 (7 days in plasma).

FIG. 6: Biodistribution of IRDye 800CW labeled C0008 in female Balb/c carrying subcutaneous CT26 tumors: Infra-red in vivo imaging of mice carrying subcutaneous CT26 tumors. Infra-red images of mice were taken 1 h, 6 h, 24 h, 48 h, 72 h, 96 h and 168 h post injection of A: IRDye 800CW labeled C0008 (5 mg/kg) or B: without treatment (scale bar applies to A and B); C: tumor penetration and retention of IRDye 800CW labeled C0008 quantified by digital image analysis.

FIG. 7: Chromatography profiles demonstrating reduced aggregation and hydrophobicity of the newly designed anti-oxMIF antibodies. (A) Comparison of elution profiles of C0008 (control antibody, gray area) and newly designed antibodies on a Superdex 200 increase 10/300 GL gel filtration column using 5×PBST as mobile phase. (B) Comparison of elution profiles of C0008 (control antibody, gray area) and newly designed antibodies on a Enrich 650 gel filtration column using 1xPBS as mobile phase (C) Comparison of elution profiles of C0008 (control antibody, gray area) and newly designed antibodies on a HiTrap Butyl HP HIC column.

FIG. 8: Binding curves of newly designed anti-oxMIF antibodies to immobilized MIF (KD determination). C0008 was used as reference antibody.

FIG. 9: Differential binding of the newly designed antibodies to oxMIF vs. redMIF. C0008 was used as reference antibody.

FIG. 10: Improved production of newly designed anti-oxMIF antibody C0083 vs. control antibody C0008.

FIG. 11: Tumor penetration of newly designed anti-oxMIF antibody C0083 vs. C0008 by infra-red in vivo imaging of mice carrying subcutaneous CT26 tumors. A: Infra-red images of mice were taken 1 h, 6 h, 24 h, 48 h, 72 h, 96 h and 168 h post injection of the IRDye 800CW labeled C0083 dosed at 5 mg/kg; B: tumor penetration and retention of IRDye 800CW labeled C0083 quantified by digital image analysis. C: comparison of tumor penetration and retention of IRDye 800CW labeled C0083 vs. C0008 quantified by digital image analysis.

FIG. 12: Purity and integrity analysis of DOTA-C0083; A: DOTA-C0083 SE-HPLC profile (λ=280 nm). B: Coomassie stained SDS PAGE: 1: molecular weight standard; 2: C0083 non-reduced; 3: C0083 reduced; 5: DOTA-C0083 non-reduced; 6: DOTA-C0083 reduced.

FIG. 13: Determination of the number of DOTA molecules grafted to C0083 determined by ITLC: ITLC separation of DOTA-C0083 which was incubated with a mixture of tracer amounts of radioactive 177Lu and stable lutetium (175Lu) in 0.04 N HCl at 2 (lane 1), 4 (lane 2), 6 (lane 3) or 8 (lane 4) fold excess of 177/175Lu in proportion to the amount of DOTA conjugated antibody.

FIG. 14: Analysis of radiolabeled ¹⁷⁷Lu-DOTA-C0083 by ITLC and autoradiography: Autoradiogram of ITLC film (¹⁷⁷Lu-DOTA-C0083 after DTPA addition; Peak 1/2 reflects free ¹⁷⁷Lu-DTPA and ¹⁷⁷Lu-DOTA-C0083 derivatives, Peak 3 reflects ¹⁷⁷Lu-DOTA-C0083).

FIG. 15: Plasma stability of ¹⁷⁷Lu-DOTA-C0083 (autoradiography): Autoradiogram of ¹⁷⁷Lu-DOTA-C0083 incubated in mouse plasma for 7 days; 1: ¹⁷⁷Lu-DOTA-C0083 derivatives and free ¹⁷⁷Lu; 2: ¹⁷⁷Lu-DOTA-C0083.

FIG. 16: Plasma stability of ¹⁷⁷Lu-DOTA-C0083 (SDS-PAGE): Reduced SDS PAGE with ¹⁷⁷Lu-DOTA-C0083 in mouse plasma (left: Coomassie staining; right: Autoradiography). Lane 1: molecular weight standard; Lane 2: ¹⁷⁷Lu-DOTA-C0083 in buffer (TO); Lane 3: ¹⁷⁷Lu-DOTA-C0083 (TO hours in plasma); Lane 4: ¹⁷⁷Lu-DOTA-C0083 (1 day in plasma), Lane 5: ¹⁷⁷Lu-DOTA-C0083 (3 days in plasma); Lane 6: ¹⁷⁷Lu-DOTA-C0083 (7 days in plasma).

FIG. 17: Amino acid sequences

FIG. 18: Chromatography profiles demonstrating reduced aggregation and hydrophobicity of the newly designed Fc silenced anti-oxMIF antibodies. (A) Comparison of elution profiles of C0008 (control antibody, gray area) and newly designed Fc silenced antibodies C0115 and C0118 on a Enrich 650 gel filtration column using 1xPBS as mobile phase (B) Comparison of elution profiles of C0008 (control antibody, gray area) and newly designed antibodies Fc silenced C0115 and C0118 and their parent antibodies C0083 and C0090 (without Fc silencing) on a HiTrap Butyl HP HIC column.

FIG. 19: Binding curves of newly designed Fc silenced anti-oxMIF antibodies to immobilized oxMIF (KD determination). Anti-oxMIF antibodies were detected by an anti-human-IgG (Fc)-HRP conjugate, C0008 was used as reference antibody.

FIG. 20: Differential binding of the newly designed Fc silenced antibodies to oxMIF vs. redMIF. C0008 was used as reference antibody and an Isotype IgG as negative control.

FIG. 21: Reduced unspecific binding of the newly designed Fc silenced anti-oxMIF antibodies to A2780 MIF^{-/-} cells determined by FACS. Staining of A2780 MIF^{-/-} cells with the newly designed anti-oxMIF antibody C0118 and its parent antibody C0090 (A) and C0115 and its parent antibody C0083 (B) and the control antibody C0008 as well as an isotype IgG as negative control; GeoMean (mean fluorescence intensity for AF488) of viable cells is plotted against antibody concentration.

FIG. 22: Strongly reduced ADCC effector function of the newly designed Fc silenced antibodies C0115 & C0118 determined by reporter assays. ADCC reporter bioassay with the newly designed Fc silenced antibodies C0115 and/or C0118 using engineered Jurkat effector cells stably expressing FcyRIIIA and HCT116-pMIF target cells compared to their parent antibodies C0083 and C0090 with wt Fc. Mean and SEM are shown (n=2).

FIG. 23: Tumor penetration and retention of the newly designed Fc silenced anti-oxMIF antibody C0115 and the reference antibody C0008 by infra-red in vivo imaging of mice carrying subcutaneous HCT116 tumors. (A) Infra-red images of mice were taken 1 h, 6 h, 24 h, 48 h, 72 h, 96 h and 168 h post injection of the IRDye 800CW labeled antibodies C0115 (upper panel) and C0008 (lower panel) dosed at 5 mg/kg; (B) tumor penetration and retention of C0115 and C0008 quantified by digital image analysis.

FIG. 24: Tumor penetration and retention of the newly designed Fc silenced anti-oxMIF antibodies C0115 and C0118 conjugated to DFO* and radiolabeled by 89Zr by in vivo PET/CT imaging of mice carrying subcutaneous CT26 or HCT116 tumors. PET and CT images of mice were taken 4-, 7- and 10-days post injection of the ⁸⁹Zr-DFO* labeled antibodies dosed at ˜10 MBq per mouse; Representative digital vertical cross sections of the mice are shown, and the white arrows mark the tumor.

### DETAILED DESCRIPTION

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (4th Ed.), Vols. 1-3, Cold Spring Harbor Laboratory Press (2012); Krebs et al., “Lewin's Genes XI”, Jones & Bartlett Learning, (2017), and Murphy & Weaver, “Janeway's Immunobiology” (9th Ed., or more recent editions), Taylor & Francis Inc, 2017.

The subject matter of the claims specifically refers to artificial products or methods employing or producing such artificial products, which may be variants of native (wild-type) products. Though there can be a certain degree of sequence identity to the native structure, it is well understood that the materials, methods and uses of the invention, e.g., specifically referring to isolated nucleic acid sequences, amino acid sequences, fusion constructs, expression constructs, transformed host cells and modified proteins, are “man-made” or synthetic, and are therefore not considered as a result of “laws of nature”.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

As used herein and in the claims, the singular form, for example “a”, “an” and “the” includes the plural, unless the context clearly dictates otherwise.

As used herein, amino acids refer to twenty naturally occurring amino acids encoded by sixty-one triplet codons. These 20 amino acids can be split into those that have neutral charges, positive charges, and negative charges: The “neutral” amino acids are shown below along with their respective three-letter and single-letter code and polarity: Alanine (Ala, A; nonpolar, neutral), Asparagine (Asn, N; polar, neutral), Cysteine (Cys, C; nonpolar, neutral), Glutamine (Gln, Q; polar, neutral), Glycine (Gly, G; nonpolar, neutral), Isoleucine (IIe, I; nonpolar, neutral), Leucine (Leu, L; nonpolar, neutral), Methionine (Met, M; nonpolar, neutral), Phenylalanine (Phe, F; nonpolar, neutral), Proline (Pro, P; nonpolar, neutral), Serine (Ser, S; polar, neutral), Threonine (Thr, T; polar, neutral), Tryptophan (Trp, W; nonpolar, neutral), Tyrosine (Tyr, Y; polar, neutral), Valine (Val, V; nonpolar, neutral), and Histidine (His, H; polar, positive (10%) neutral (90%)).

The “positively” charged amino acids are: Arginine (Arg, R; polar, positive), and Lysine (Lys, K; polar, positive).

The “negatively” charged amino acids are: Aspartic acid (Asp, D; polar, negative), and Glutamic acid (Glu, E; polar, negative).

The term “immunoconjugate” refers to a conjugate of an anti-oxMIF antibody or antigen binding fragment thereof and a second moiety, and the term “radioimmunoconjugate” refers to a conjugate of an anti-oxMIF antibody or antigen binding fragment thereof and a radioisotope (radionuclide).

The radioisotope can be a beta, alpha or positron emitting radionuclide including but not limited to ¹¹C, ¹³N, ¹⁵O, ¹⁸F, ³²P, ⁶⁴Cu, ⁶⁷Cu, ⁶⁷Ga, ⁸⁹Zr, ⁹⁰Y, ⁹⁹mTc, ¹⁰³Pd, ¹⁰⁵Rh, ¹⁰⁹Pd, ¹¹¹Ag, ¹¹¹In, ¹²³I, ¹²⁴I, ¹²⁵I, ¹³¹I, ¹⁴⁰La, ¹⁴⁹Tb, ¹⁴⁹Pm, ¹⁵³Sm, ¹⁵⁹Gd, ¹⁶⁵Dy, ¹⁶⁶Dy, ¹⁶⁶Ho, ¹⁶⁹Yb, ¹⁷⁵Yb, ¹⁷⁷Lu, ²²⁷Th, ¹⁸⁶Re, ¹⁸⁸Re, ¹⁹²Ir, ¹⁹³mPt, ¹⁹⁵mPt, ¹⁹⁸Au, ¹⁹⁹Au, ²¹¹At, ²¹²Pb, ²¹²Bi, ²¹³Bi, ²²⁵Ac, ²²³Ra, ²²⁷Th, specifically the radioisotope is ⁶⁷Ga, ⁸⁹Zr, ¹¹¹In, ¹²⁴I, ¹³¹I, ¹⁷⁷Lu, ²²⁵Ac.

Useful alpha emitting radionuclides may be, but are not limited to ¹⁴⁹Tb, ²¹¹At, ²¹²Bi, ²¹³Bi and ²²⁵Ac.

A specifically useful beta transmitter is ¹⁷⁷Lu.

Positron emission or beta plus decay (β⁺ decay) is a subtype of radioactive decay called beta decay, in which a proton inside a radionuclide nucleus is converted into a neutron while releasing a positron and an electron neutrino (Vₑ). Positron emitting radionuclides are used in positron emission tomography (PET).

A specifically useful positron emitter is ¹¹C, ¹³N, ¹⁵O, ¹⁸F, or ⁸⁹Zr, specifically it is ⁸⁹Zr.

Any type of linker with sufficient complexing ability and a functional group allowing direct or indirect conjugation to a protein or a peptide could be used. Examples of such linkers for attaching the radioisotope to the antibody are described in the literature (e.g. Brechbiel M. W., 2008; Liu S., 2008, Zeglis & Lewis 2011). The radionuclides in the present invention are preferably conjugated to the anti-oxMIF antibody either directly or by using bifunctional chelating agents. These could be cyclic, linear or branched chelating agents. Particular reference may be made to the polyaminopolyacid chelators which comprise a linear, cyclic or branched polyazaalkane backbone with acidic (e.g. carboxyalkyl) groups attached at backbone nitrogens.

Some useful non-limiting examples are cyclic chelators or bifunctional chelating agents thereof like diethylenetriamine pentaacetic acid (DTPA), and 1,4,7,10-tetra-azacylcododecane-N,N′, N″, N′″-tetra acetic acid, ca-DTPA, ibca-DTPA, 1B4M-DTPA, lys-DTPA, vinyl DTPA, glu-DTPA, p-SCN-bn-DOTA, DOTA-NHS-ester, deferoxamine B or derivatives thereof; or linear chelators or bifunctional chelating agents thereof like p-SCN-Bn-DTPA, HOPO and CHX-A″-DTPA, ethylenediamine tetraacetic acid (EDTA), DTPA, EDTMP, NOTA, TETA, DOTMP, N2S2, N3S, HEDP. Examples of further suitable chelating agents include DOTA derivatives such as p-isothiocyanatobenzyl-1,4,7,10-tetraazacyclododecane-1,4,7,10-tetraacetic acid (p-SCN-Bz-DOTA) and DTPA derivatives such as p-isothiocyanatobenzyl-diethylenetriaminepentaacetic acid (p-SCN-Bz-DTPA), the first being cyclic chelators, the latter being linear chelators.

Further examples of deferoxamine (desferrioxamine, DFO) and derivatives thereof include, but are not limited to p-NCS-Bz-DFO, DFOSq, DFO*, oxoDFO*, DFO*Sq, DFO*-NCS, DFO*pPhe-NCS.

Purification can follow to remove unconjugated chelator, and after reaction of the chelator antibody conjugate with the radionuclide, purification can be performed to remove any unconjugated radionuclide. Alternatively, the chelator and the radionuclide can be combined firstly and subsequently conjugated to the antibody.

The radiolabeling procedure may be more convenient if the chelator is conjugated to the antibody before the radiolabeling takes place. The principles of preparing radiolabeled conjugates using chelators attached to antibodies is described broader in e.g. Liu S., 2008.

The antibody or antigen-binding fragment of the radioimmunoconjugate comprises at least one binding site specifically recognizing oxMIF. Such antibodies have been described by Kerschbaumer R. et al., 2012. The anti-oxMIF antibody is specific for a β-sheet structure within MIF including a highly conserved catalytic motif (57Cys-Ala-Leu-Cys60, SEQ ID NO: 44) of the thiol protein oxidoreductase, which is linked to the biologic function of MIF (Kerschbaumer R. et al., 2012). CDRs specific to oxMIF are described herein.

Imalumab (C0008) CDRs comprise SEQ ID NOs. 7 to 12.

Alternatively, the CDRs of further antibodies specifically recognizing oxMIF specifically comprise SEQ ID NOs 1 to 6 (Bax8), or SEQ ID NOs 13 to 18 (Bax74), or SEQ ID NOs 19 to 24 (Bax94), or SEQ ID NOs 22, 23, 24, 26, 27 and 21 (BaxA10), or any sequences having at least 80%, 90%, 95% sequence identity with SEQ ID NOs 7 to 12, SEQ ID NOs 1 to 6, SEQ ID NOs 13 to 18, or SEQ ID NOs 19 to 24, or SEQ ID NOS 22, 23, 24, 26, 27 and 21.

Reduced aggregation propensity and/or hydrophobicity is particularly important in the case of radioimmunoconjugates, since any aggregates and/or highly hydrophobic interfaces might lead to non-specific attachment of the radioimmunoconjugates, and thus of the radionuclides, to non-tumor tissue.

In a specific embodiment, the anti-oxMIF antibody or antigen binding fragment thereof of the radioimmunoconjugate exhibits reduced aggregation propensity and reduced hydrophobicity due to targeted amino acid substitutions in the variable heavy and light chain domains in comparison to the unmodified antibody lacking said amino acid substitutions.

Reduction of aggregation potential and reduced hydrophobicity is due to amino acid substitutions at selected positions within the variable domains of the antibody described herein.

The level of antibody aggregation can be measured using a variety of known techniques including mass spectrometry, size exclusion chromatography (SEC), dynamic light scattering (DLS), light obscuration (LO), dynamic imaging particle analysis (DIP A) techniques such as microflow imaging (MFI), and Coulter counter (CC), differential scanning fluorometry (DSF).

Reduced hydrophobicity and reduced aggregation potential as used herein refer to a reduction of the surface hydrophobicity and a reduced aggregation potential of the inventive antibody of the radioimmunoconjugate compared to the surface hydrophobicity and aggregation potential of Imalumab comprising SEQ ID NO: 29 (VH), and SEQ ID NO: 32 (VL) and SEQ ID NO: 37 (CH1-CH3), SEQ ID NO: 41 (CL). The sequence of C0008 contains the sequence of Imalumab, published in the Proposed INN List 111 (WHO Drug Information, Vol. 28, No. 2, 2014), but lacking the C-terminal lysine. Measurement can be performed using a variety of known techniques including but not limited to hydrophobic interaction chromatography (HIC) or affinity-capture self-interaction nanoparticle spectroscopy (AC-SINS, Estep P. et al., 2015).

In an embodiment, the oxMIF antibody of the radioimmunoconjugate having reduced aggregation potential and reduced hydrophobicity specifically comprises:
a light chain variable domain having 1, 2, 3, 4, or 5 of the amino acid substitutions M30L, F49Y, A51G, P80S, W93F with reference to SEQ ID NO: 32 and according to the Kabat numbering, or
a light chain variable domain with at least 95, specifically at least 96, 97, 98 or 99% sequence identity to SEQ ID NO: 32 comprising the natural tyrosine at position 36 and furthermore 1, 2, 3, 4, or 5 of the amino acid substitutions M30L, F49Y, A51G, P80S, and/or W93F, either
in combination with a heavy chain variable domain comprising SEQ ID NO: 29, or
in combination with a heavy chain variable domain comprising the amino acid substitutions L5Q and/or W97Y with reference to SEQ ID NO: 29 and according to the numbering of Kabat, or
a heavy chain variable domain comprising SEQ ID NO: 29 with at least 95, specifically at least 96, 97, 98 or 99% sequence identity and further comprising at least one of the amino acid substitutions L5Q or W97Y.

In an alternative embodiment, the light chain variable domain comprising SEQ ID NO: 32 has 1, 2, 3, 4, 5 amino acid substitutions with the proviso that the natural tyrosine at position 36 is preserved and that furthermore 1, 2, 3, 4, or 5 of the amino acids are substituted at positions M30, F49, A51, P80, W93.

In a further embodiment, the anti-oxMIF antibody of the radioimmunoconjugate has reduced aggregation potential and reduced hydrophobicity and specifically comprises:
a heavy chain variable domain comprising SEQ ID NO: 29 and amino acid substitution W97Y or amino acid substitutions L5Q and W97Y, or
a heavy chain variable domain comprising SEQ ID NO: 29 with 1, 2, 3, 4 or 5 amino acid substitutions and further comprising amino acid substitution W97Y, optionally in combination with L5Q.

The natural tyrosine at position 36 of the light chain is kept unmodified to preserve the binding properties of the anti-oxMIF antibody. Any modifications at said amino acid position may result in unwanted impaired binding properties.

The radioimmunoconjugate as described herein specifically comprises a light chain variable domain with one of the following amino acid substitution combinations: M30L, F49Y, A51G, P80S, and W93F; F49Y, A51G, and W93F; F49Y and A51G with reference to the amino acid numbering of SEQ ID NO: 32.

The radioimmunoconjugate of the present invention may comprise any of the following variable light and heavy chain domain combinations:
SEQ ID NO: 33 and SEQ ID NO: 30
SEQ ID NO: 34 and SEQ ID NO: 29
SEQ ID NO: 34 and SEQ ID NO: 30
SEQ ID NO: 34 and SEQ ID NO: 31
SEQ ID NO: 35 and SEQ ID NO: 29
SEQ ID NO: 35 and SEQ ID NOs: 30, 31
SEQ ID NO: 36 and SEQ ID NO: 29
SEQ ID NO: 36 and SEQ ID NOs: 30, 31.

In a further embodiment, the anti-oxMIF antibody comprises any one of the following sequence combinations:
SEQ ID NOs: 37, 30, 33, and 41,
SEQ ID NOs: 37, 31, 33, and 41,
SEQ ID NOs: 37, 29, 34, and 41,
SEQ ID NOs: 37, 30, 34, and 41,
SEQ ID NOs: 37, 31, 34, and 41,
SEQ ID NOs: 37, 29, 35, and 41,
SEQ ID NOs: 37, 30, 35, and 41,
SEQ ID NOs: 37, 31, 35, and 41,
SEQ ID NOs: 37, 29, 36, and 41,
SEQ ID NOs: 37, 30, 36, and 41, or
SEQ ID NOs: 37, 31, 36, and 41.

In a specific embodiment, the anti-oxMIF antibodies of the present radioimmunoconjugate show reduced binding to Fcγ receptor bearing cells (decreased binding to FcγRI, FcγRII, FcγRIII) due to amino acid substitutions at selected positions in the heavy chain constant region, specifically in the CH2 region.

By “effector function” as used herein is meant a biochemical event that results from the interaction of an antibody Fc region with an Fc receptor or ligand. Effector functions include but are not limited to ADCC, ADCP, and CDC.

By “effector cell” as used herein is meant a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and T cells, and may be from any organism including but not limited to humans, mice, rats, rabbits, and monkeys. According to the invention, the anti-oxMIF antibodies described herein have silenced effector functions due to amino acid substitutions at selected positions in the heavy chain constant region, specifically in the Fc region. Decreased or silenced effector functions of these antibodies due to reduced complement- and FcγR-mediated activities can include reduced or abolished complement dependent cytotoxicity (CDC), antibody-dependent cell-mediated cytotoxicity (ADCC) and/or antibody dependent cellular phagocytosis (ADCP).

The term “Fc” or “Fc region” (or fragment crystallizable region) as used herein refers to the polypeptide comprising the constant region of an antibody excluding the first constant region immunoglobulin domain (CH1 domain) and in some cases, part of the hinge. The Fc region refers to the C-terminal region of an antibody. The Fc region is composed of two identical protein fragments, derived from the second and third constant domains of the antibody's two heavy chains: Chain A and Chain B. The second and third constant domains are known as the CH2 domain and the CH3 domain, respectively. The CH2 domain comprises a CH2 domain sequence of Chain A and a CH2 domain sequence of Chain B. The CH3 domain comprises a CH3 domain sequence of Chain A and a CH3 domain sequence of Chain B. As used herein, the Fc region includes the hinge region or a part thereof.

The “CH2 domain” of a human IgG Fc region sequence usually extends from about amino acid 231 to about amino acid 340 according to EU numbering. The CH2 domain sequence is unique in that it is not closely paired with another domain sequence. Rather, two N-linked branched carbohydrate chains are interposed between the two CH2 domain sequences of an intact native IgG molecule.

The “CH3 domain” comprises the stretch of residues C-terminal to a CH2 domain sequence in an Fc region sequence (i.e. from about amino acid residue 341 to about amino acid residue 447 of an IgG according to EU numbering).

A “functional Fc region” possesses the “effector functions” of a native Fc region. Exemplary “effector functions” include C1 q binding; complement dependent cytotoxicity; Fc receptor binding; antibody-dependent cell-mediated cytotoxicity (ADCC); etc. Such effector functions generally require the Fc region to be combined with a binding domain (e.g. an antibody variable domain) and can be assessed using various assays known in the art and as herein disclosed.

A “native Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region and native sequence human IgG3 Fc region as well as naturally occurring variants thereof.

A “variant Fc region” comprises an amino acid sequence which differs from that of a native Fc region sequence by virtue of “one or more amino acid substitutions”. The variant Fc region sequence has at least one amino acid substitution compared to a native Fc region sequence or to the Fc region sequence of a parent polypeptide, e.g. from about one to about twenty amino acid substitutions, and preferably from about one to about seventeen amino acid substitutions in a native Fc region sequence or in the Fc region sequence of the parent polypeptide. In certain embodiments, the variant Fc region sequence herein possesses at least about 80% identity with a native Fc region sequence and/or with an Fc region sequence of a parent polypeptide, and most preferably at least about 90% identity therewith, more preferably at least about 95% identity therewith.

In a specific embodiment, the amino acid substitutions are at any one of positions E233, L234, L235, G236, G237, P238, D265, S267, H268, N297, S298, T299, E318, L328, P329, A330, P331 with reference to IgG1 according to EU numbering. These silencing mutations, however, can also be introduced into the Fc of wild type IgG2, IgG3 or IgG4 at corresponding positions according to EU numbering.

Modification of the Fc region resulting in decreased or silenced Fc are known in the art and are described in Saunders K., 2019 and Liu R. et al., 2020.

In an alternative embodiment, the amino acid substitutions are at any one or all of positions L234 F, H268Q, K274Q, Y296F, A327G, A330S, P331S in the CH2 domain and R355Q, K409R, Q419E, P445L in the CH3 domain.

Specifically, the Fc silenced anti-oxMIF antibody described herein comprises one or more of the following combinations of amino acid substitutions or deletions:
i) L235, G237 and E318, specifically L235A, G237A and E318A;
ii) L234, L235, specifically L234A, L235A;
iii) S228, L235, specifically S228P, L235E;
iv) G236, L328, specifically G236R, L328R;
v) S298, T299, specifically S298G, T299A;
vi) L234, L235, P331, specifically L234F, L235E, P331S;
vii) H268, V309, A330, P331, specifically H268Q, V309L, A330S, P331S;
viii) E233, L234, L235, G236, S267, specifically E233P, L234V, L235A, G236del, S267K;
ix) L234, L235, P329, specifically L234A, L235A, P329G;
x) V234, G237, P238, H268, A330, P331, specifically V234A, G237A, P238S, H268A, A330S, P331S;
xi) L234, L235, D265, specifically L234F, L235E, D265A;
xii) D265, specifically D265A;
xiii) G237, specifically G237A;
xiv) E318, specifically E318A;
xv) E233, specifically E233P,
xvi) G236, L328, specifically G236R, L328R;
xvii) L235, specifically L235E;
xviii) L234, L235, P331, specifically L234Q, L235F, P331S;
xix) L234, L235, G237, P238, H268, A330, P331, specifically L234A, L235A, G237A, P238S, H268A, A330S, P331S.
xx) N297, specifically N297A, N297Q or N297G, leading to an aglycosylated antibody

Glycosylation, O- and N-glycosylation, is a post-translational modification of Abs, which can be regulated by a range of B cell stimuli, including environmental factors, such as stress or disease, cytokine activity, and innate immune signaling receptors, such as Toll-like receptors. Glycosylation pattern of the parent antibody can be modified by methods well known in the art. Specifically, O-linked glycosylation sites are located in the CH2 and hinge region.

The term “aglycosylated” indicates that the Fc region is not glycosylated. All human constant regions of the IgG isotype are known to be glycosylated at the asparagine residue at position 297, which makes up part of the N-glycosylation motif Asparagine 297-X 298-Serine 299 or Threonine 299, where X is the residue of any amino acid except proline. The glycan has a heptasaccharide core and variable extensions, such as fucose, galactose and/or sialic acid. The antibody of the invention may thus be aglycosylated by the replacement of Asparagine 297 in such a constant region with another amino acid which cannot be glycosylated or deglycosylated by enzymatic means. Any other amino acid residue can potentially be used, but alanine is the most preferred. Alternatively, glycosylation at Asparagine 297 can be prevented by altering one of the other residues of the motif, e.g. by replacing residue 298 by proline, or residue 299 by any amino acid other than serine or threonine. Techniques for performing this site directed mutagenesis are well known to those skilled in the art and may for example be performed using a commercially available site directed mutagenesis kit.

The term “silenced Fc” refers to an Fc region of an antibody whose effector function is reduced or eliminated due to amino acid substitutions or modification of the glycosylation pattern resulting in modified glycan that reduce or eliminate binding of the antibody to any of FcγR, such as FcγRIIaH, FcγRIIaR, FcγRIIb FcγRIIIaF, FcγRIIIaV, and FcγRIa receptors and to complement factor C1 q protein. Such reduction or elimination of this binding results in reduction or elimination of effector functions typically mediated by the wild-type IgG Fc region.

If FcγR binding, such as one any FcγRIIaH, FcγRIIaR, FcγRIIb FcγRIIIaF, FcγRIIIaV, and FcγRIa receptors and to complement factor C1 q protein is completely abolished, the term “Fc null” may be used herein.

A great extent of Fc silencing can be achieved by combining mutations. L234 and L235, these residues being located close to the hinge area and reducing FcγR binding when substituted by alanine. As an example, the combination of L234A and L235A with P329G can lead to a near complete inhibition of FcγR interaction for all isoforms.

A Fc silenced anti-oxMIF antibody or an antigen binding fragment thereof with greatly reduced, silenced, negligible or ablated FcγR binding affinity and C1 q binding affinity is one which has diminished FcγR binding activity and C1 q binding activity compared to a parent polypeptide or to a polypeptide comprising a native Fc region sequence. In some embodiments, an Fc silenced anti-oxMIF antibody or an antigen binding fragment thereof with greatly reduced, silenced, negligible or ablated FcγR binding affinity and C1 q binding affinity has also greatly reduced, silenced, negligible or ablated ADCC, ADCP and CDC activity compared to a parent polypeptide or to a polypeptide comprising a native Fc region sequence. A Fc silenced anti-oxMIF antibody or an antigen binding fragment thereof which displays decreased or undetectable binding to FcγR may bind all FcγRs with lower affinity than the parent polypeptide. Such variants which display decreased binding to an FcγR may possess little or no appreciable binding to an FcγR. In one specific embodiment, the variant displays 0-20% binding to the FcγR compared to a native IgG Fc region, e.g. as measured by change in equilibrium constant. In one embodiment, the variant displays 0-10% binding to the FcγR compared to a native IgG Fc region. In one embodiment, the variant displays 0-5% binding to the FcγR compared to a native IgG Fc region. In one embodiment, the variant displays 0-1% binding to the FcγR compared to a native IgG Fc region.

Antibodies described herein with silenced complement activities can be determined by cell-based CDC assays and reduced or abolished binding to C1q determined by i.e. SPR or ELISA.

Decreased or silenced CDC activity is determined to be at least 1.5-fold, specifically at least 2-fold, 5-fold, 6-fold, 7-fold, 8-fld, 9-fold, more specifically at least 10-fold downregulated compared to a reference, i.e. unmodified, wild type antibody, e.g. C0008.

Decreased ADCC or ADCP activity is determined to be at least 2-fold, 5-fold, 6-fold, 7-fold, 8-fold, 9-fold, more specifically at least 10-fold decreased potent compared to a reference antibody, i.e. unmodified, wild type antibody, e.g. C0008.

The Fc domain of the radioimmunoconjugate antibody part exhibiting decreased FcγR binding compared to a radioimmunoconjugate comprising the wildtype IgG1 Fc region may specifically comprise an Fc variant domain of a wild-type human IgG1 constant domain (SEQ ID NO: 37), comprising 1, 2, 3, 4, 5, 6, or 7 or more amino acid substitutions, more specifically 1, 2 or 3 amino acid substitutions at any one of positions, L234, L235, G236, G237, N297, L328 or P329, more specifically comprising substitutions at positions L234 and L235.

More specifically, the anti-oxMIF antibody part comprises an Fc variant domain of a wild-type human IgG1 constant domain (SEQ ID NO: 37), comprising amino acid substitutions at positions L234 and L235, specifically it is L234A and L235A.

In a further aspect the reduction or down-modulation of FcγR binding by the anti-oxMIF antibody of the radioimmunoconjugate comprising the Fc variant, is a reduction to 0, 2.5, 5, 10, 20, 50 or 75% of the value observed for the anti-oxMIF antibody comprising the wildtype Fc region.

In vitro assays can be conducted to confirm the reduction/depletion of FcγR binding. For example, Fc receptor (FcR) binding assays can be conducted to ensure that the antibody lacks FcγR binding. Such assays can include but are not limited to SPR binding studies or FACS on cells specifically expressing FcγRs.

In a further embodiment, the anti-oxMIF antibody comprises any one of the following sequence combinations:
SEQ ID NOs: 38, 30, 33, and 41,
SEQ ID NOs: 38, 31, 33, and 41,
SEQ ID NOs: 38, 29, 34, and 41,
SEQ ID NOs: 38, 30, 34, and 41,
SEQ ID NOs: 38, 31, 34, and 41,
SEQ ID NOs: 38, 29, 35, and 41,
SEQ ID NOs: 38, 30, 35, and 41,
SEQ ID NOs: 38, 31, 35, and 41,
SEQ ID NOs: 38, 29, 36, and 41,
SEQ ID NOs: 38, 30, 36, and 41, or
SEQ ID NOs: 38, 31, 36, and 41.

In a specific embodiment, the anti-oxMIF antibodies of the present radioimmunoconjugate show reduced binding to the Neonatal Receptor (FcRn) due to amino acid substitutions at selected positions in the heavy chain constant region.

FcRn is expressed by endothelial cells, which internalize serum components including soluble IgGs from the bloodstream by pinocytosis. IgG binding to FcRn is pH-dependent; the acidic pH (pH 6.0) inside the endosomal compartment allows the IgGs to bind to FcRn. After recycling back to the cell surface, the IgG dissociates from FcRn at physiological pH (˜pH 7.2), is released back into the blood circulation and thereby protected from lysosomal degradation, leading to prolonged half-life of IgGs. Decreased binding of the radioimmunoconjugate to the FcRn leads to reduced half-life in the circulation and faster in vivo clearance.

FcRn binding and in vivo clearance/half-life determinations can also be performed using methods known in the art (see, e.g., Petkova, S. B., et al., 2006).

The Fc domain of the radioimmunoconjugate antibody part exhibiting decreased FcRn binding compared to a radioimmunoconjugate comprising the wildtype IgG1 Fc region may preferably comprise an Fc variant domain showing a reduced FcγR binding (SEQ ID NO: 38) and the Fc-portion of the radioimmunoconjugate may further comprising one, two or three amino acid substitutions at any one of positions I253, H310, and H435.

In a further embodiment, the anti-oxMIF antibody comprises any one of the following sequence combinations:
SEQ ID NOs: 39, 30, 33, and 41,
SEQ ID NOs: 39, 31, 33, and 41,
SEQ ID NOs: 39, 29, 34, and 41,
SEQ ID NOs: 39, 30, 34, and 41,
SEQ ID NOs: 39, 31, 34, and 41,
SEQ ID NOs: 39, 29, 35, and 41,
SEQ ID NOs: 39, 30, 35, and 41,
SEQ ID NOs: 39, 31, 35, and 41,
SEQ ID NOs: 39, 29, 36, and 41,
SEQ ID NOs: 39, 30, 36, and 41,
SEQ ID NOs: 39, 31, 36, and 41.

In a further embodiment, the anti-oxMIF antibody comprises any one of the following sequence combinations
SEQ ID NOs: 40, 30, 33, and 41,
SEQ ID NOs: 40, 31, 33, and 41,
SEQ ID NOs: 40, 29, 34, and 41,
SEQ ID NOs: 40, 30, 34, and 41,
SEQ ID NOs: 40, 31, 34, and 41,
SEQ ID NOs: 40, 29, 35, and 41,
SEQ ID NOs: 40, 30, 35, and 41,
SEQ ID NOs: 40, 31, 35, and 41,
SEQ ID NOs: 40, 29, 36, and 41,
SEQ ID NOs: 40, 30, 36, and 41, or
SEQ ID NOs: 40, 31, 36, and 41.

The oxMIF binding site of the antibody described herein is specific for the oxidized form of MIF, i.e. for animal, specifically for mammalian oxMIF, such as but not limited to mouse, rat, monkey and human, specifically for human oxMIF, but does not show substantial cross-reactivity to reduced MIF. oxMIF is the disease-related structural isoform of MIF which can be specifically and predominantly detected in tumor tissue of cancer patients. In one embodiment, the humanized or human anti-oxMIF binding site comprises one or more (e.g., all three) light chain complementary determining regions of a humanized or human anti-oxMIF binding domain described herein, such as the CDRs comprised in e.g. SEQ ID NOs: 32, 33, 34, 35 or 36 and one or more (e.g., all three) heavy chain complementary determining regions of a humanized or human anti-oxMIF binding domain described herein, e.g. SEQ ID NOs: 29, 30, or 31.

oxMIF binding specificity of the radioimmunoconjugate can be determined by any assay appropriate for determining selective binding to oxMIF, such as any competition assay against an unlabeled and nonspecific control antibody, such as unlabeled Imalumab, with respect to binding to oxMIF.

The term “antibody” herein is used in the broadest sense and encompasses polypeptides or proteins that consist of or comprise antibody domains, which are understood as constant and/or variable domains of the heavy and/or light chains of immunoglobulins, with or without a linker sequence. The term encompasses various antibody structures, including but not limited to monoclonal antibodies, polyclonal antibodies, multispecific antibodies such as bispecific antibodies, trispecific antibodies, and antibody fragments as long as they exhibit the desired antigen-binding activity, i.e. binding to oxMIF. The term also encompasses fusion proteins, such as fusions with immunotoxins or antibody conjugates, such as antibody drug conjugates binding to oxMIF. Antibodies also encompass full-length antibody formats with IgG structures in which the Fc region has been replaced by an Fcab™ containing second distinct antigen binding site.

Antibody domains may be of native structure or modified by mutagenesis or derivatization, e.g. to modify the antigen binding properties or any other property, such as stability or functional properties, such as binding to the Fc receptors, such as FcRn and/or Fc-gamma receptors, preferably modifications reducing binding to Fc-gamma receptors and/or FcRn. Polypeptide sequences are considered to be antibody domains, if comprising a beta-barrel structure consisting of at least two beta-strands of an antibody domain structure connected by a loop sequence.

It is understood that the term “antibody” includes antigen binding derivatives and fragments thereof. A derivative is any combination of one or more antibody domains or antibodies of the invention and/or a fusion protein in which any domain of the antibody of the invention may be fused at any position of one or more other proteins, such as other antibodies or antibody formats, e.g. a binding structure comprising CDR loops, a receptor polypeptide, but also ligands, scaffold proteins, enzymes, labels, toxins and the like.

The term “antibody” shall particularly refer to polypeptides or proteins that exhibit binding properties to the target antigen oxMIF.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, single chain antibody molecules (e.g. scFv), Fab-scFv fusion, Fab-(scFv)₂, (scFv)₂, F(ab′)₂, diabodies, cross-Fab fragments; linear antibodies. In a specific embodiment, the antibody fragment of the radioimmunoconjugate is fused to a silenced Fc-portion or silenced Fc-domains by a hinge region.

In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full-length antibodies.

Antibody fragments as referred herein can also encompass silenced Fc domains comprising one or more structural loop regions containing antigen binding regions such as Fcab™ or full-length antibody formats with IgG structures in which the silenced Fc region has been replaced by an Fcab™ containing second distinct antigen binding site.

The term “functional variant” or “functionally active variant” also includes naturally occurring allelic variants, as well as mutants or any other non-naturally occurring variants. As is known in the art, an allelic variant, or also referred to as homologue, is an alternate form of a nucleic acid or peptide that is characterized as having a substitution, deletion, or addition of one or more nucleotides or amino acids that does essentially not alter the biological function of the nucleic acid or polypeptide. Specifically, a functional variant may comprise a substitution, deletion and/or addition of 1, 2, 3, 4, 5, 6, 7, 8, 9, 10, 11, 12, 13, 14, 15, 16, 17, 18, 19 or 20 amino acid residues, or a combination thereof, which substitutions, deletions and/or additions are conservative modifications and do not alter the antigen binding properties. Specifically, a functional variant as described herein comprises no more than or up to 1, 2, 3, 4, 5, 6, 7, 8, 9, 10, 11, 12, 13, 14, 15, 16, 17, 18, 19 or 20 amino acid substitutions, deletions and/or additions, which are conservative modifications and do not alter the antibody's function. Specifically, a functionally active variant as described herein comprises up to 15, preferably up to 10 or 5, amino acid substitutions, deletions and/or additions, which are conservative modifications and do not alter the antibody's function.

Functional variants may be obtained by sequence alterations in the polypeptide or the nucleotide sequence, e.g. by one or more point mutations, wherein the sequence alterations retain or improve a function of the unaltered polypeptide or the nucleotide sequence, when used in combination of the invention. Such sequence alterations can include, but are not limited to, (conservative) substitutions, additions, deletions, mutations and insertions.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc. Such conservative substitutions may be for example isoleucine for leucine, aspartate for glutamate, cysteine for serine etc.

Non-conservative substitutions are those that take place within amino acids that are not related in their side chains and chemical properties, such as but not limited to alanine for proline or valine, or cysteine for phenylalanine, phenylalanine for serine or valine etc.

A point mutation is particularly understood as the engineering of a polynucleotide that results in the expression of an amino acid sequence that differs from the non-engineered amino acid sequence in the substitution or exchange, deletion or insertion of one or more single (non-consecutive) or doublets of amino acids for different amino acids. Preferred point mutations refer to the exchange of amino acids of the same polarity and/or charge. In this regard, amino acids refer to twenty naturally occurring amino acids encoded by sixty-one triplet codons. These 20 amino acids can be split into those that have neutral charges, positive charges, and negative charges.

As used herein, “Fab fragment or Fab” refers to an antibody fragment comprising a light chain fragment comprising a VL domain and a constant domain of a light chain (CL), and a VH domain and a first constant domain (CH1) of a heavy chain. The antibodies of the invention can comprise at least one Fab fragment, wherein either the variable regions or the constant regions of the heavy and light chain are exchanged. Due to the exchange of either the variable regions or the constant regions, said Fab fragment is also referred to as “cross-Fab fragment” or “crossover Fab fragment”. Two different chain compositions of a crossover Fab molecule are possible and comprised in the antibodies of the invention: The variable regions of the Fab heavy and light chain can be exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab (VLVH).

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker can have the following orders in N-terminal to C-terminal direction: VH-CH1-linker-VL-CL, VL-CL-linker-VH-CH1, VH-CL-linker-VL-CH1 or VL-CH1-linker-VH-CL; and wherein said linker is a polypeptide of at least 20 amino acids, at least 30 amino acids, specifically between 32 and 50 amino acids. Said single chain Fab fragments VH-CH1-linker-VL-CL, VL-CL-linker-VH-CH1, VH-CL-linker-VL-CH1 and VL-CH1-linker-VH-CL, can be stabilized via the natural disulfide bond between the CL domain and the CH1 domain. In addition, these single chain Fab molecules might be further stabilized by generation of interchain disulfide bonds via insertion of cysteine residues.

An Fv fragment is the smallest unit of immunoglobulin molecule with function in antigen-binding activities. A “single-chain variable fragment” or “scFv” is a fusion protein of the variable regions of the heavy (V_{H}) and light chains (V_{L}) of immunoglobulins, connected with a short linker peptide or a disulfide bond. The linker is usually rich in glycine for flexibility, as well as serine or threonine for solubility, and can either connect the N-terminus of the V_{H} with the C-terminus of the V_{L}, or vice versa. Single-chain variable fragments lack the constant Fc region. ScFv retains the specificity of the original immunoglobulin, despite removal of the constant regions and the introduction of the linker.

The term “N-terminus” denotes the last amino acid of the N-terminus.

The term “C-terminus” denotes the last amino acid of the C-terminus.

The term “minibody” refers to an antibody which is composed of a pair of single-chain Fv fragments which are linked via CH3 domains (single chain Fv-CH3), Specifically, the minibody is of about 75 kDa.

The term “diabody” refers to a noncovalent dimer of single-chain Fv (scFv) fragment that consists of the heavy chain variable (V_{H}) and light chain variable (V_{L}) regions connected by a small peptide linker. Another form of diabody is single-chain (Fv)₂ in which two scFv fragments are covalently linked to each other. In addition, by tandem linking genes in each chain with internal linker, four VH and VL domains can be expressed in tandem and folded as single chain diabody (scDb), which is also an effective strategy for bispecific antibody production. Furthermore, fusing recombinant variable domains to an Fc region or CH3 domain (scDb-Fc and scDb-CH3, diabody-CH3) can double the valency of the final product. The increased size can also prolong the half-life of diabody in serum. Specifically, the diabody-CH3 is of about 125 kDa.

According to a specific embodiment, the antibodies described herein may comprise one or more tags for purification and/or detection, such as but not limited to affinity tags, solubility enhancement tags and monitoring tags.

Specifically, the affinity tag is selected from the group consisting of poly-histidine tag, poly-arginine tag, peptide substrate for antibodies, chitin binding domain, RNAse S peptide, protein A, ß-galactosidase, FLAG tag, Strep II tag, streptavidin-binding peptide (SBP) tag, calmodulin-binding peptide (CBP), glutathione S-transferase (GST), maltose-binding protein (MBP), S-tag, HA tag, and c-Myc tag, specifically the tag is a His tag comprising four or more H, such as a hexahistidine tag.

By “fused” or “connected” is meant that the components (e.g. a Fab molecule and an Fc domain subunit) are linked by peptide bonds, either directly or via one or more peptide linkers.

The term “linker” as used herein refers to a peptide linker and is preferably a peptide with an amino acid sequence with a length of 2, 3, 4, 5, 6, 7 or more amino acids, preferably with a length of 2-10, more preferably of 3-5 amino acids.

The term “immunoglobulin” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N-to C-terminus, each heavy chain has a variable region (VH), also called a variable heavy domain or a heavy chain variable domain, followed by three constant domains (CH1, CH2, and CH3), also called a heavy chain constant region. Similarly, from N-to C-terminus, each light chain has a variable region (VL), also called a variable light domain or a light chain variable domain, followed by a constant light (CL) domain, also called a light chain constant region. An immunoglobulin of the IgG class essentially consists of two Fab molecules and an Fc domain, linked via the immunoglobulin hinge region. The heavy chain of an immunoglobulin may be assigned to one of five types, called α (IgA), σ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ).

The term “chimeric antibody” refers to an antibody in which a portion of the heavy and/or light chain is derived from a particular source or species, while the remainder of the heavy and/or light chain is derived from a different source or species, usually prepared by recombinant DNA techniques. Chimeric antibodies may comprise a rabbit or murine variable region and a human constant region. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art (Morrison, S. L., et al., 1984).

A “human antibody” possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. The term “human antibody” as used herein also comprises such antibodies which are modified in the constant region e.g. by “class switching” i.e. change or mutation of Fc parts (e.g. from IgG1 to IgG4 and/or IgG1/lgG4 mutation.) Other forms of humanized antibodies encompassed by the present invention are those in which the constant region has been additionally modified or changed from that of the original antibody to generate the new properties, e.g. in regard to C1q binding and/or Fc receptor (FcR) binding.

The term “recombinant human antibody”, as used herein, is intended to include all human antibodies that are prepared, expressed, created or isolated by recombinant means, such as antibodies isolated from a host cell such as a HEK cell, NS0 or CHO cell or from an animal (e.g. a mouse) that is transgenic for human immunoglobulin genes or antibodies expressed using a recombinant expression vector transfected into a host cell. The amino acid sequences of the VH and VL regions of the recombinant antibodies are sequences that, while derived from and related to human germ line sequences, may not naturally exist within the human antibody repertoire in vivo.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as described in Kabat et al., 1991.

The term “antigen” as used herein interchangeably with the term “target” or “target antigen” shall refer to a whole target molecule or a fragment of such molecule recognized by an antibody binding site. Specifically, substructures of an antigen, e.g. a polypeptide or carbohydrate structure, generally referred to as “epitopes”, e.g. B-cell epitopes or T-cell epitope, which are immunologically relevant, may be recognized by such binding site.

The term “epitope” as used herein shall in particular refer to a molecular structure which may completely make up a specific binding partner or be part of a specific binding partner to a binding site of an antibody format of the present invention. An epitope may either be composed of a carbohydrate, a peptidic structure, a fatty acid, an organic, biochemical or inorganic substance or derivatives thereof and any combinations thereof. If an epitope is comprised in a peptidic structure, such as a peptide, a polypeptide or a protein, it will usually include at least 3 amino acids, specifically 5 to 40 amino acids, and specifically less than 10 amino acids, specifically between 4 to 10 amino acids. Epitopes can be either linear or conformational epitopes. A linear epitope is comprised of a single segment of a primary sequence of a polypeptide or carbohydrate chain. Linear epitopes can be contiguous or overlapping. Conformational epitopes are comprised of amino acids or carbohydrates brought together by folding the polypeptide to form a tertiary structure and the amino acids are not necessarily adjacent to one another in the linear sequence. Such oxMIF epitope may be sequence EPCALCS (SEQ ID NO: 45 located within the central region of oxMIF.

The term “antigen binding domain” or “binding domain” or “binding-site” refers to the part of an antigen binding moiety that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more antibody variable domains (also called antibody variable regions). Preferably, an antigen binding domain comprises an antibody light chain variable region (VL) and an antibody heavy chain variable region (VH).

The term “binding site” as used herein with respect to the antibody of the present invention refers to a molecular structure capable of binding interaction with an antigen. Typically, the binding site is located within the complementary determining region (CDR) of an antibody, herein also called “a CDR binding site”, which is a specific region with varying structures conferring binding function to various antigens. The varying structures can be derived from natural repertoires of antibodies, e.g. murine or human repertoires, or may be recombinantly or synthetically produced, e.g. by mutagenesis and specifically by randomization techniques. These include mutagenized CDR regions, loop regions of variable antibody domains, in particular CDR loops of antibodies, such as CDR1, CDR2 and CDR3 loops of any of VL and/or VH antibody domains. The antibody format as used according to the invention typically comprises one or more CDR binding sites, each specific to an antigen.

The term “specific” as used herein shall refer to a binding reaction which is determinative of the cognate ligand of interest in a heterogeneous population of molecules. Herein, the binding reaction is at least with an oxMIF antigen. Thus, under designated conditions, e.g. immunoassay conditions, the antibody that specifically binds to its particular target does not bind in a significant amount to other molecules present in a sample, specifically it does not show substantial cross-reactivity to reduced MIF.

A specific binding site is typically not cross-reactive with other targets. Still, the specific binding site may specifically bind to one or more epitopes, isoforms or variants of the target, or be cross-reactive to other related target antigens, e.g., homologs or analogs.

The specific binding means that binding is selective in terms of target identity, high, medium or low binding affinity or avidity, as selected. Selective binding is usually achieved if the binding constant or binding dynamics to a target antigen such as oxMIF is at least 10-fold different, preferably the difference is at least 100-fold, and more preferred a least 1000-fold compared to binding constant or binding dynamics to an antigen which is not the target antigen.

The term “valent” as used within the current application denotes the presence of a specified number of binding sites in an antibody molecule. The valency of antibody refers to the number of antigenic determinants that an individual antibody molecule can bind. As such, the terms “bivalent”, “tetravalent”, and “hexavalent” denote the presence of two binding sites, four binding sites, and six binding sites, respectively, in an antibody molecule.

The greater an antibody's valency (number of antigen binding sites), the greater the amount of antigen it can bind.

The term “monovalent” as used herein with respect to a binding site of an antibody shall refer to a molecule comprising only one binding site directed against a target antigen.

The antibody of the present invention is understood to comprise a monovalent, bivalent, tetravalent or multivalent binding site specifically binding oxMIF

The term “hypervariable region” or “HVR,” as used herein refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition (Kabat et al., 1991) Hypervariable regions (HVRs) are also referred to as complementarity determining regions (CDRs), and these terms are used herein interchangeably in reference to portions of the variable region that form the antigen binding regions. The exact residue numbers which encompass a particular CDR will vary depending on the sequence and size of the CDR. Those skilled in the art can routinely determine which residues comprise a particular CDR given the variable region amino acid sequence of the antibody.

Kabat defined a numbering system for variable region sequences that is applicable to any antibody. One of ordinary skill in the art can unambiguously assign this system of “Kabat numbering” to any variable region sequence, without reliance on any experimental data beyond the sequence itself. The Kabat numbering of residues can be determined for a given antibody by alignment at regions of homology of the sequence of the antibody with a “standard” Kabat numbered sequence. As used herein, “Kabat numbering” refers to the numbering system set forth by Kabat et al., 1983, U.S. Dept. of Health and Human Services, “Sequence of Proteins of Immunological Interest”. Unless otherwise specified, references to the numbering of specific amino acid residue positions in an antibody variable region are according to the Kabat numbering system. The numbering of the constant region is according to EU numbering index.

CDRs also comprise “specificity determining residues,” or “SDRs,” which are residues that contact antigen. SDRs are contained within regions of the CDRs called abbreviated-CDRs, or a-CDRs. Unless otherwise indicated, HVR residues and other residues in the variable domain (e.g., FR residues) are numbered herein according to Kabat et al., supra.

“Percent (%) sequence identity” with respect to the polypeptide sequences identified herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

According to the present invention, sequence identity of the variable or constant region sequences is at least 95%, 96%, 97%, 98%, 99%, 99.5% or 100% with the respective sequences described herein. Due to the specification in %, it could be that, purely arithmetically, the amino acids or nucleic acids can no longer be specified as whole numbers. As an example, calculation of identity would result in 1.5 amino acids being identical or non-identical. In this case, the values are rounded up so that the nucleic- and amino acids are always given as integers.

As used herein the term “subject” or “patient” or “individual” refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human, most preferably a human suspected of having abnormal cells, including malignant cells in a tumor.

An “isolated” nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-oxMIF” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

“No substantial cross-reactivity” means that a molecule (e.g., an antibody) does not recognize or specifically bind an antigen different from the actual target antigen of the molecule (e.g. an antigen closely related to the target antigen), specifically reduced MIF, particularly when compared to that target antigen. For example, an antibody may bind less than about 10% to less than about 5% to an antigen different from the actual target antigen, or may bind said antigen different from the actual target antigen at an amount consisting of less than about 10%, 9%, 8%7%, 6%, 5%, 4%, 3%, 2%, 1%, 0.5%, 0.2%, or 0.1%, preferably less than about 2%, 1%, or 0.5%, and most preferably less than about 0.2% or 0.1% to an antigen different from the actual target antigen. Binding can be determined by any method known in the art such as, but not limited to ELISA or surface plasmon resonance.

The recombinant production of the antibody of the invention preferably employs an expression system, e.g. including expression constructs or vectors comprising a nucleotide sequence encoding the antibody format.

The term “expression system” refers to nucleic acid molecules containing a desired coding sequence and control sequences in operable linkage, so that hosts transformed or transfected with these sequences are capable of producing the encoded proteins. In order to effect transformation, the expression system may be included on a vector; however, the relevant DNA may then also be integrated into the host chromosome. Alternatively, an expression system can be used for in vitro transcription/translation.

“Expression vectors” used herein are defined as DNA sequences that are required for the transcription of cloned recombinant nucleotide sequences, i.e. of recombinant genes and the translation of their mRNA in a suitable host organism. Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The terms “plasmid” and “vector” as used herein include autonomously replicating nucleotide sequences as well as genome integrating nucleotide sequences.

Specifically, the term refers to a vehicle by which a DNA or RNA sequence (e.g. a foreign gene), e.g. a nucleotide sequence encoding the antibody format of the present invention, can be introduced into a host cell, so as to transform the host and promote expression (e.g. transcription and translation) of the introduced sequence. Plasmids are preferred vectors of the invention.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”. A common type of vector is a “plasmid”, which generally is a self-contained molecule of double-stranded DNA that can readily accept additional (foreign) DNA and which can readily be introduced into a suitable host cell. A vector of the invention often contains coding DNA and expression control sequences, e.g. promoter DNA, and has one or more restriction sites suitable for inserting foreign DNA. Coding DNA is a DNA sequence that encodes a particular amino acid sequence for a particular polypeptide or protein such as an antibody format of the invention. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors of the invention will often include one or more replication systems for cloning or expression, one or more markers for selection in the host, e.g. antibiotic resistance, and one or more expression cassettes.

The procedures used to ligate DNA sequences, e.g. providing or coding for the factors of the present invention and/or the protein of interest, a promoter, a terminator and further sequences, respectively, and to insert them into suitable vectors containing the information necessary for integration or host replication, are well known to persons skilled in the art, e.g. described by Sambrook et al, 2012.

A host cell is specifically understood as a cell, a recombinant cell or cell line transfected with an expression construct, such as a vector according to the invention.

The term “host cell line” as used herein refers to an established clone of a particular cell type that has acquired the ability to proliferate over a prolonged period of time. The term host cell line refers to a cell line as used for expressing an endogenous or recombinant gene to produce polypeptides, such as the recombinant antibody format of the invention.

A “production host cell” or “production cell” is commonly understood to be a cell line or culture of cells ready-to-use for cultivation in a bioreactor to obtain the product of a production process, the recombinant antibody format of the invention. The host cell type according to the present invention may be any prokaryotic or eukaryotic cell.

The term “recombinant” as used herein shall mean “being prepared by genetic engineering” or “the result of genetic engineering”, e.g. specifically employing heterologous sequences incorporated in a recombinant vector or recombinant host cell.

An antibody may be produced using any known and well-established expression system and recombinant cell culturing technology, for example, by expression in bacterial hosts (prokaryotic systems), or eukaryotic systems such as yeasts, fungi, insect cells or mammalian cells. An antibody molecule of the present invention may be produced in transgenic organisms such as a goat, a plant or a transgenic mouse, an engineered mouse strain that has large fragments of the human immunoglobulin loci and is deficient in mouse antibody production. An antibody may also be produced by chemical synthesis.

According to a specific embodiment, the host cell is a production cell line of cells selected from the group consisting of CHO, PerC6, CAP, HEK, HeLa, NSO, SP2/0, hybridoma and Jurkat. More specifically, the host cell is obtained from CHO cells.

The host cell of the invention is specifically cultivated or maintained in a serum-free culture, e.g. comprising other components, such as plasma proteins, hormones, and growth factors, as an alternative to serum.

Host cells are most preferred, when being established, adapted, and completely cultivated under serum free conditions, and optionally in media which are free of any protein/peptide of animal origin.

Anti-oxMIF antibodies of the radioimmunoconjugate as described herein can be recovered from the culture medium using standard protein purification methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. Some examples of pharmaceutically acceptable carriers are water, saline, phosphate buffered saline, amino acids such as glycine or histidine, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

As used herein, “treatment”, “treat” or “treating” or “therapy” refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The anti-oxMIF antibody of the radioimmunoconjugate of the invention and the pharmaceutical compositions comprising it, can be administered in combination with one or more other therapeutic, diagnostic or prophylactic agents. Additional therapeutic agents include other anti-neoplastic, antitumor, anti-angiogenic, chemotherapeutic agents, steroids, or checkpoint inhibitors depending on the disease to be treated. The pharmaceutical compositions of this invention may be in a variety of forms, for example, liquid solutions (e.g., injectable and infusible solutions), dispersions or suspensions and liposomes. Various other delivery systems are also known and can be used to administer the radioimmunoconjugate described herein, e.g. encapsulation in liposomes, microparticles, microcapsules, receptor-mediated endocytosis (see, e.g., Wu and Wu, 1987).

The amount of the radioimmunoconjugate composition of the invention which will be effective in the treatment, prevention or management of cancer or in the diagnosis of cancer can be determined by standard research techniques. For example, the dosage of the composition which will be effective in the treatment, prevention or management of cancer or in the diagnosis of cancer can be determined by administering the composition to an animal model such as, e.g., the animal models disclosed herein or known to those skilled in the art. In addition, in vitro assays may optionally be employed to help identify optimal dosage ranges. Selection of the preferred effective dose can be determined (e.g., via clinical trials) by a skilled artisan based upon the consideration of several factors which will be known to one of ordinary skill in the art. Such factors include the disease to be treated or prevented, the symptoms involved, the patient's body mass, the patient's immune status and other factors known by the skilled artisan to reflect the accuracy of administered pharmaceutical compositions. The precise dose to be employed in the formulation will also depend on the route of administration, and the seriousness of the cancer, and should be decided according to the judgment of the practitioner and each patient's circumstances. Effective doses may be extrapolated from dose-response curves derived from in vitro or animal model test systems.

In a specific embodiment, the therapeutically effective amount of the radioimmunoconjugate is in the range of about 25 mCi to 250 mCi, 50 mCi to 200 mCi, 75 mCi to 175 mCi, or 100 mCi to 150 mCi.

The radioimmunoconjugate can be administered in combination with known chemotherapeutic agents. Examples of chemotherapeutic agents, include but are not limited to, BCNU, cisplatin, gemcitabine, hydroxyurea, paclitaxel, temozomide, topotecan, fluorouracil, vincristine, vinblastine, procarbazine, dacarbazine, altretamine, cisplatin, methotrexate, mercaptopurine, thioguanine, fludarabine phosphate, cladribine, pentostatin, fluorouracil, cytarabine, azacitidine, vinblastine, vincristine, etoposide, teniposide, irinotecan, docetaxel, doxorubicin, daunorubicin, dactinomycin, idarubicin, plicamycin, adriamycin, mitomycin, bleomycin, tamoxifen, flutamide, leuprolide, goserelin, aminoglutethimide, anastrozole, amsacrine, asparaginase, mitoxantrone, mitotane and amifostine.

The preferred formulation depends on the intended mode of administration and therapeutic application. Typical preferred compositions are in the form of injectable or infusible solutions, such as compositions similar to those used for passive immunization of humans. The preferred mode of administration is parenteral (e.g., intradermal, intravenous, subcutaneous, epidural, intraperitoneal, intramuscular). In a preferred embodiment, the radioimmunoconjugate is administered by intravenous infusion or injection. As will be appreciated by the skilled artisan, the route and/or mode of administration will vary depending upon the desired results.

In a specific embodiment, it may be desirable to administer the radioimmunoconjugate locally to the area in need of treatment; this may be achieved by, for example, and not by way of limitation, local infusion during surgery, by injection, by means of a catheter, by means of a suppository, or by means of an implant, said implant being of a porous, non-porous, or gelatinous material, including membranes, such as silastic membranes, or fibers.

In yet another embodiment, the radioimmunoconjugate or any compositions comprising the radioimmunoconjugate can be delivered in a controlled release system. In one embodiment, a pump may be used. In another embodiment, polymeric materials can be used. In yet another embodiment, a controlled release system can be placed in proximity of the therapeutic target, e.g., the brain, thus requiring only a fraction of the systemic dose.

The radioimmunoconjugate may be administered once, but more preferably is administered multiple times.

The invention also relates to compositions comprising the radioimmunoconjugate, for the treatment and diagnosis of a subject in need of treatment for MIF-related conditions, specifically (hyper)proliferative disorders. In some embodiments, the subject in need of treatment is a human.

The term “cancer” as used herein refers to proliferative diseases, specifically to non-solid and solid cancers. Examples of cancer include but are not limited to, carcinoma, lymphoma, blastoma, sarcoma, and leukemia. More particular, examples of such cancers include squamous cell cancer, small-cell lung cancer, non-small cell lung cancer, adenocarcinoma of the lung, squamous carcinoma of the lung, cancer of the peritoneum, myeloma (e.g., multiple myeloma), hepatocellular cancer, gastrointestinal cancer, pancreatic cancer, glioblastoma/glioma (e.g., anaplastic astrocytoma, glioblastoma multiforme, anaplastic oligodendroglioma, anaplastic oligodendroastrocytoma), Ewing's sarcoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, melanoma, squamous cell carcinoma (SCC) (e.g., head and neck, esophageal, and oral cavity), colorectal adenocarcinoma, medullary thyroid cancer, papillary thyroid cancer, astrocytic tumor, neuroblastoma, cervical cancer, endometrial carcinoma, and malignant seminoma, including refractory versions of any of the above cancers, or a combination of one or more of the above cancers.

Hyperproliferative disorders, such as cancerous diseases or cancer, that may be treated and/or diagnosed by the radioimmunoconjugates of the invention can involve any tissue or organ and include but are not limited to brain, lung, squamous cell, bladder, gastric, pancreatic, breast, head, neck, liver, renal, ovarian, prostate, colorectal, esophageal, gynecological, nasopharynx, or thyroid cancers, melanomas, lymphomas, leukemias or multiple myelomas. In particular, radioimmunoconjugates of the invention are useful to treat and/or diagnose carcinomas of the ovary, pancreas, colon and lung and hematological malignancies.

In a specific embodiment, the antibodies of the radioimmunoconjugate are highly suitable for the treatment and/or diagnosis of cancerous diseases, specifically for the treatment of solid tumors are recombinant anti-oxMIF antibodies or antigen binding fragments thereof having reduced aggregation potential and reduced hydrophobicity, comprising
i.) the following variable domains:
   (a) a light chain variable domain comprising SEQ ID NO: 32 and at least one of the amino acid substitutions M30L, F49Y, A51G, P80S, W93F, or a functional variant of SEQ ID NO: 32 with 1, 2, 3, 4, or 5 amino acid substitutions, further comprising a conserved tyrosine at position 36 and further comprising at least one of the amino acid substitutions M30L, F49Y, A51G, P80S, W93F; and one of
   (b) a heavy chain variable domain comprising SEQ ID NO: 29; or
   (c) a heavy chain variable domain comprising SEQ ID NO: 29 and at least one of the amino acid substitutions L5Q or W97Y, or a functional variant of SEQ ID NO: 29 comprising SEQ ID NO: 29 with 1, 2, 3, 4 or 5 amino acid substitutions and further comprising at least one of the amino acid substitutions L5Q or W97Y.

Furthermore, in another preferred aspect, the anti-oxMIF antibody of the radioimmunoconjugate comprises modifications that reduce FcγR binding which can be, for example, identified by an alanine scan of the Fc. Specifically, it comprises an Fc variant domain of a wild-type human IgG1 constant domain having SEQ ID NO: 37, comprising one, two or three amino acid substitutions at any one of positions, L234, L235, G236, G237, N297, L328 or P329, and wherein said radioimmunoconjugate exhibits decreased FcγR binding compared to a radioimmunoconjugate comprising the wildtype IgG1 Fc region. In a further embodiment, the radioimmunoconjugate comprises an Fc variant domain with reduced FcγR binding (SEQ ID NO: 38), comprising one, two or three amino acid substitutions at any one of positions, 1253, H310, H435, and wherein said radioimmunoconjugate exhibits reduced affinity to the human FcRn compared to a radioimmunoconjugate comprising the wildtype IgG1 Fc region.

The radioimmunoconjugates of the invention can be used as diagnostic or detectable agents. Radioimmunoconjugates of the invention can be useful for monitoring or prognosing the development or progression of a cancer as part of a clinical testing procedure, such as determining the efficacy of a particular therapy. Additionally, such radioimmunoconjugates can be useful for monitoring or prognosing the development or progression of cancerous conditions.

In another embodiment, following initial administration of the radioimmunoconjugate of the invention, the cancer cells can be imaged and the relative amount or number of cancerous cells can be determined by any available means. The invention includes diagnostic methods to detect cancer and/or assess the effect therapeutic agents on cancer cells in an organ or body area of a patient.

The present methods include administration of a composition comprising a detectable amount of an anti-oxMIF antibody conjugated to a radioisotope to a patient before and after therapy. Subsequent to administration of the therapeutic agent, an additional amount of detectable anti-oxMIF antibody can be administered to determine the relative amount of cancer cells remaining following treatment. Comparison of the before and after treatment images can be used as a mean to assess the efficacy of the treatment wherein a decrease in the number of cancer cells imaged following treatment is indicative of an efficacious treatment regimen.

As used herein, the term “detectable amount” refers to the amount of radioimmunoconjugate, which binds to oxMIF, administered to a patient that is sufficient to enable detection of binding of the labeled antibody to one or more malignant cancer cells in a tumor. As used herein, the term “imaging effective amount” refers to the amount of the radioimmunoconjugate administered to a patient that is sufficient to enable imaging of binding of the anti-oxMIF antibody to one or more malignant cancer cells in a tumor.

The methods of the invention comprise the radioimmunoconjugate of the invention which, in conjunction with non-invasive imaging techniques such as gamma imaging such as positron emission tomography (PET) or single-photon emission computed tomography (SPECT), are used to identify and quantify abnormal cells in vivo including malignant cells in tumors. The term “in vivo imaging” refers to any method which permits the detection of the radioimmunoconjugate as described above. For gamma imaging, the radiation emitted from the tumor or area being examined is measured and expressed either as total binding, or as a ratio in which total binding in one tissue is normalized to (for example, divided by) the total binding in another tissue or the entire body of the same subject during the same in vivo imaging procedure. Total binding in vivo is defined as the entire signal detected in a tumor or tissue by an in vivo imaging technique without the need for correction by a second injection of an identical quantity of labeled compound along with a large excess of unlabeled, but otherwise chemically identical compound.

For purposes of in vivo imaging, the type of detection instrument available is a major factor in selecting a given label. For instance, radioactive isotopes are particularly suitable for in vivo imaging in the methods described herein. The type of instrument used will guide the selection of the radioisotope. For instance, the radioisotope chosen must have a type of decay detectable by a given type of instrument. Another consideration relates to the half-life of the radioisotope. The half-life should be long enough so that it is still detectable at the time of maximum uptake by the target, but short enough so that the host does not sustain deleterious radiation. The isotopically-labeled radioimmunoconjugate can be detected using gamma imaging where emitted gamma irradiation of the appropriate wavelength is detected. Methods of gamma imaging include, but are not limited to, positron emission tomography (PET) imaging or for single photon emission computerized tomography (SPECT). Preferably, for SPECT detection, the chosen radiolabel will lack a particulate emission, but will produce a large number of photons. For PET detection, the radiolabel will be a positron-emitting radioisotope which will be detected by the PET camera.

In the present invention, radioimmunoconjugates are useful for in vivo detection and imaging of tumors. These compounds are to be used in conjunction with non-invasive imaging techniques such as positron emission tomography (PET), single-photon emission computed tomography (SPECT) or Cerenkov luminescence imaging (CLI). In accordance with this invention, the immunoconjugate may be labeled (complexed) with any acceptable radioisotope.

According to a further embodiment, the radioimmunoconjugate is used for determining the localisation of cancer cells, cells of the tumor microenvironment or a tumor in a subject in need thereof comprising administering the radioimmunoconjugate to said subject and detecting the radioimmunoconjugate by in vivo SPECT and PET imaging.

According to a further embodiment, the radioimmunoconjugate is used for imaging one or more cancer cells, cells of the tumor microenvironment, organs or tissues in a subject, comprising administering the radioimmunoconjugate to said subject and detecting the radioimmunoconjugate by in vivo PET or SPECT imaging.

According to a further embodiment, the radioimmunoconjugate is used for determination and (relative) quantification of oxMIF on cancer cells, cells of the tumor microenvironment, organs or tissues in a subject, comprising administering the radioimmunoconjugate to said subject and detecting the radioimmunoconjugate by in vivo PET or SPECT imaging.

Generally, the dosage of the radioimmunoconjugate will vary depending on considerations such as age, condition, sex, and extent of disease in the patient, contraindications, if any, concomitant therapies and other variables, to be adjusted by the skilled artisan. Dosage can vary from the range of about 25 mCi to 250 mCi for therapeutic administration and radiation dose may be in the range of between 0.5 and 10 mCi, such as about 1 to 5 mCi for diagnostic purposes. Individual dosages can be determined by the skilled person.

Administration to the patient may be local or systemic and accomplished intravenous, intraarterial, intra-thecal (via the spinal fluid), intra-cranial or the like. Administration may also be intra-dermal or intra-cavitary, depending upon the body site under examination.

After a sufficient time has elapsed for the radioimmunoconjugate to bind with the abnormal cells, for example about 30 minutes to 48 hours, or to 72 hours, or even longer, the area of the subject under investigation is examined by routine imaging techniques such as SPECT, planar scintillation imaging, PET, and emerging imaging techniques, as well. The exact protocol will necessarily vary depending upon factors specific to the patient, as noted above, and depending upon the body site under examination, method of administration and type of label used; the determination of specific procedures would be routine to the skilled artisan. For tumor imaging, preferably, the amount (total or specific binding) of the bound radioimmunoconjugate is measured and compared (as a ratio) with the amount of radioimmunoconjugate bound to the tumor following chemotherapeutic treatment.

In another embodiment, the radioimmunoconjugate of the invention can be used for diagnosis and prognosis by using tissues and fluids distal to the primary tumor site (as well as methods using tissues and fluids of the primary tumor and/or tissue and fluids surrounding the tumor). Radioimmunoconjugates of the invention can be used to assay oxMIF levels in a biological sample using classical immunohistological methods as known to those of skilled in the art (e.g., see Jalkanen et al. (1985) J. Cell. Biol. 101, 976-985; and Jalkanen et al. (1987) J. Cell. Biol. 105, 3087-3096).

In still further embodiments, the present invention provides diagnostic kits, including both immunodetection and imaging kits, for use with the immunodetection and imaging methods described above.

The present invention also encompasses the following items:
1. A radioimmunoconjugate comprising a recombinant anti-oxMIF antibody or an antigen binding fragment thereof, comprising
   (a1) a light chain variable domain comprising SEQ ID NO: 32 having at least one of amino acid substitutions M30L, F49Y, A51G, P80S, W93F, or
   (a2) a light chain variable domain comprising SEQ ID NO: 32 with 1, 2, 3, 4, or 5 amino acid substitutions further comprising
      a conserved tyrosine at position 36, and
      at least one of amino acid substitutions M30L, F49Y, A51G, P80S, W93F; and
      (b1) a heavy chain variable domain comprising SEQ ID NO: 29; or
      (b2) a heavy chain variable domain comprising SEQ ID NO: 29 and the amino acid substitutions L5Q and/or W97Y, or
      (b3) a heavy chain variable domain comprising SEQ ID NO: 29 having at least one of amino acid substitutions L5Q or W97Y, and 1, 2, 3, 4 or 5 further amino acid substitutions;
      wherein amino acid positions are numbered according to Kabat,
      wherein said antibody or antigen binding fragment thereof has reduced aggregation potential and reduced hydrophobicity compared to an antibody or an antigen binding fragment thereof comprising SEQ ID NO: 32 and SEQ ID NO: 29 lacking the amino acid substitutions.
2. The radioimmunoconjugate of item 1, wherein the light chain variable domain comprises the amino acid substitution W93F and the heavy chain variable region comprises the amino acid substitution W97Y.
3. The radioimmunoconjugate of item 1 or 2, wherein the radioisotope is selected from the group consisting of ¹¹C, ¹³N, ¹⁵O, ¹⁸F, ³²P, ⁶⁴Cu, ⁶⁷Cu, ⁶⁷Ga, ⁸⁹Zr, ⁹⁰Y, ⁹⁹mTc, ¹⁰³Pd, ¹⁰⁵Rh, ¹⁰⁹Pd, ¹¹¹Ag, ¹¹¹In, ¹²³I, ¹²⁴I, ¹²⁵I, ¹³¹I, ¹⁴⁰La, ¹⁴⁹Tb, ¹⁴⁹Pm, ¹⁵³Sm, ¹⁵⁹Gd, ¹⁶⁵Dy, ¹⁶⁶Dy, ¹⁶⁶Ho, ¹⁶⁹Yb, ¹⁷⁵Yb, ¹⁷⁷Lu, ²²⁷Th, ¹⁸⁶Re, ¹⁸⁸Re, ¹⁹²Ir, ¹⁹³mPt, ¹⁹⁵mPt, ¹⁹⁸Au, ¹⁹⁹Au, ²¹¹At, ²¹²Pb, ²¹²Bi, ²¹³Bi, ²²⁵Ac, ²²³Ra, and ²²⁷Th, specifically the radioisotope is selected from the group consisting of ⁶⁷Ga, ⁸⁹Zr, ¹¹¹In, ¹²⁴I, ¹³¹I, ¹⁷⁷Lu, and ²²⁵Ac.
4. The radioimmunoconjugate of item 1 or 2, comprising a Fc variant domain of a wild-type human IgG1 constant domain having SEQ ID NO: 37, comprising at least one, amino acid substitution at any one of positions, L234, L235, G236, G237, N297, L328 or P329, and wherein said radioimmunoconjugate exhibits decreased binding to Fcγ-receptors, specifically FcγRI, FcγRII, FcγRIII, compared to a radioimmunoconjugate comprising the wildtype IgG1 Fc region.
5. The radioimmunoconjugate of any one of items 1 to 4, comprising an Fc variant domain of a human IgG1 constant domain, comprising one, two or three amino acid substitutions at any one of positions, I253, H310, H435, and wherein said radioimmunoconjugate exhibits reduced affinity to the human FcRn compared to a radioimmunoconjugate comprising the wildtype IgG1 Fc region
6. The radioimmunoconjugate of any one of items 1 to 5, comprising an amino acid sequence selected from the group consisting of SEQ ID NOs: 31, 33, 34, 35, and 36.
7. The radioimmunoconjugate of any one of items 1 to 5, comprising SEQ ID NOs: 29 and 34; SEQ ID NOs: 30 and 33, SEQ ID NOs: 30 and 34, SEQ ID NOs: 31 and 34, SEQ ID NOs: 31 and 36, or SEQ ID NOs: 31 and 33, in combination with any one of SEQ ID NOs: 37, 38, 39, and 40.
8. The radioimmunoconjugate of any one of items 1 to 7, selected from the group consisting of IgG, IgG-fusion proteins, Fv, scFv, scFv fusion proteins, diabody, diabody fusion proteins, Fab, Fab-fusion proteins, scFab, scFab-fusion proteins, Fab′, and F(ab′)2, Fab′-SH, Fcab, Fcab fusion proteins, fusion proteins of two or more single chain antibodies, minibody, small immune protein (SIP) format, and nanobodies and nanobody fusion proteins.
9. The radioimmunoconjugate of any one of items 1 to 8, further comprising a chelating group, selected from the group of DOTA and deferoxamine B (DFO) and DFO*.
10. The radioimmunoconjugate of any one of items 1 to 9 for use in the preparation of a medicament.
11. A pharmaceutical composition comprising the radioimmunoconjugate of any one of items 1 to 9, optionally together with a pharmaceutical carrier or adjuvant.
12. The pharmaceutical composition of item 11, wherein said pharmaceutical composition is formulated for intravenous administration.
13. The pharmaceutical composition of items 11 or 12 for use the treatment of a patient suffering from cancer, specifically in the treatment of tumors, specifically of hematological or solid tumors, more specifically for the treatment of colorectal cancer, ovarian cancer, breast cancer, prostate cancer, pancreas cancer, and lung cancer.
14. An isolated nucleic acid encoding the recombinant anti-oxMIF antibody or antigen binding fragment thereof of the radioimmunoconjugate of any one of items 1 to 8.
15. An expression vector comprising the nucleic acid molecule(s) of item 14.
16. A host cell containing the nucleic acid of item 12 or 13 or an expression vector of item 13.
17. A method for producing the radioimmunoconjugate of any one of items 1 to 9, comprising expressing a nucleic acid encoding an anti-oxMIF antibody in a host cell and further labeling said antibody with a radioisotope.
18. Use of the radioimmunoconjugate of any one of items 1 to 9 for detection of oxMIF level in the cancer of a subject.
19. Use of the radioimmunoconjugate of any one of items 1 to 9 for diagnosis of cancer in a subject.
20. Method for in vitro diagnosing cancer, wherein the radioimmunoconjugate of any one of items 1 to 9 is used for detecting tumor cells in a sample.
21. Method for in vivo diagnosing cancer, wherein the radioimmunoconjugate of any one of items 1 to 9 is used for detecting tumor cells in a subject.
22. A kit for production of the radioimmunoconjugate of any one of items 1 to 9, comprising two or more vials, wherein one vial contains a conjugate comprising the chelator linked to an anti-oxMIF antibody and a second vial contains a radioisotope, specifically selected from 177Lu and 89Zr.
23. The kit of item 21, wherein the content of one or several of the vials is lyophilized or in solution.
24. Method for treating cancer using the radioimmunoconjugate of any one of items 1 to 9, or a pharmaceutical composition of item 11 or 12.

### Examples

The examples described herein are illustrative of the present invention and are not intended to be limitations thereon. Many modifications and variations may be made to the techniques described and illustrated herein without departing from the spirit and scope of the invention. Accordingly, it should be understood that the examples are illustrative only and are not limiting upon the scope of the invention.

### Example 1

### Generation of 177Lu-C0008-Radioimmunoconjugate

### Materials and Methods

C0008 was incubated with SCN-Bn-DOTA at a molar ratio of 1:20 in HEPES 0.3 M pH 9. The labelling mixture was stirred for 2 hours at 37° C. After the reaction, excess SCN-Bn-DOTA was removed by multiple centrifugations in 0.9% NaCl at 3000 g using 30 kDa MWCO Amicon Ultra centrifugal filters (regenerated cellulose, Millipore). The conjugates were examined by SE-HPLC to evaluate the chemical purity at the end of the purification. SE-HPLC was conducted on a Superdex 200 10/300 GL column eluted with phosphate buffer containing arginine at a flow rate of 0.6 mL/min. In addition, the purity and integrity of DOTA-C0008 was assessed by SDS-PAGE under reducing and non-reducing conditions (4-15% linear gradient polyacrylamide gel; Bio-Rad Laboratories).

The number of attached DOTA molecules per antibody was determined by using the method of Meares et al., 1984, and Instant Thin-Layer Chromatography (ITLC). A small aliquot of DOTA conjugated antibody was incubated with a mixture of tracer amounts of radioactive ¹¹¹In and stable indium (¹¹⁵In) at 2-, 4-, 6- and 8-fold excess of ^{111/115}In compared to the amount of DOTA conjugated antibody (labelling conditions are shown in Table 1). Unconsumed indium was then complexed with an excess of EDTA to prevent the formation of insoluble indium hydroxide and the nonspecific attachment of indium to the antibody. For analysis 1 μL of the radioactive solution was placed on an ITLC strip and was eluted with 0.1M citrate buffer at pH 5. Autoradiography of TLC plates was obtained after exposure on a phosphorous screen (Typhoon IP, Amersham) using ImageQuant TL according to the volume integration. The number of DOTA molecules attached per DOTA-C0008 was estimated from the labelling efficiency and the molar ratio of protein to ^{111/115}In ions added into the solution.

TABLE 1
Labeling protocol for determination
of number of chelate grafted to C0008

| | | |
|---|---|---|
| Ratio | Ratio 2 | 80 μg of DOTA-C0008 (0.512 nmol) |
| of molar | | 6 μL of NH₄Ac 250 mM pH 5.4 |
| equivalents | | 3 μL of ^{111/115}In 2 eq (1.024 nmol/1.0 μCi) |
| for ^{115/111}In/ | | 25 μL (Total volume) |
| DOTA-C0008 | Ratio 4 | 80 μg of DOTA-C0008 (0.512 nmol) |
| | | 6 μL of NH₄Ac 250 mM pH 5.4 |
| | | 3 μL of ^{111/115}In 4 eq |
| | | 25 μL (Total volume) |
| | Ratio 6 | 80 μg of DOTA-C0008 (0.512 nmol) |
| | | 6 μL of NH₄Ac 250 mM pH 5.4 |
| | | 3 μL of ^{111/115}In 6 eq |
| | | 25 μL (Total volume) |
| | Ratio 8 | 80 μg of DOTA-C0008 (0.512 nmol) |
| | | 6 μL of NH₄Ac 250 mM pH 5.4 |
| | | 3 μL of ^{111/115}In 8 eq |
| | | 25 μL (Total volume) |

In order to radiolabel DOTA-C0008 with 177Lu at a specific activity of 15 mCi/mg (555 MBq/mg), 4500 μCi (166.5 MBq) of 177Lu chloride in 0.04 N HCl (ITG Germany) and 12 μL of ammonium acetate 0.25 M, pH 5.4 were added to 300 μg of DOTA-C0008. The reaction mixture was incubated for 10 minutes at 37° C. after which the radiolabeled conjugate was incubated with 1 mM DTPA (final conc.) for additional 5 minutes to complex excess of ¹⁷⁷Lu. The chelation efficiency was evaluated by ITLC eluting in citrate buffer 0.1 M pH 5 after DTPA addition and Autoradiography of TLC plates (Typhoon IP, Amersham) using ImageQuant TL according to the volume integration.

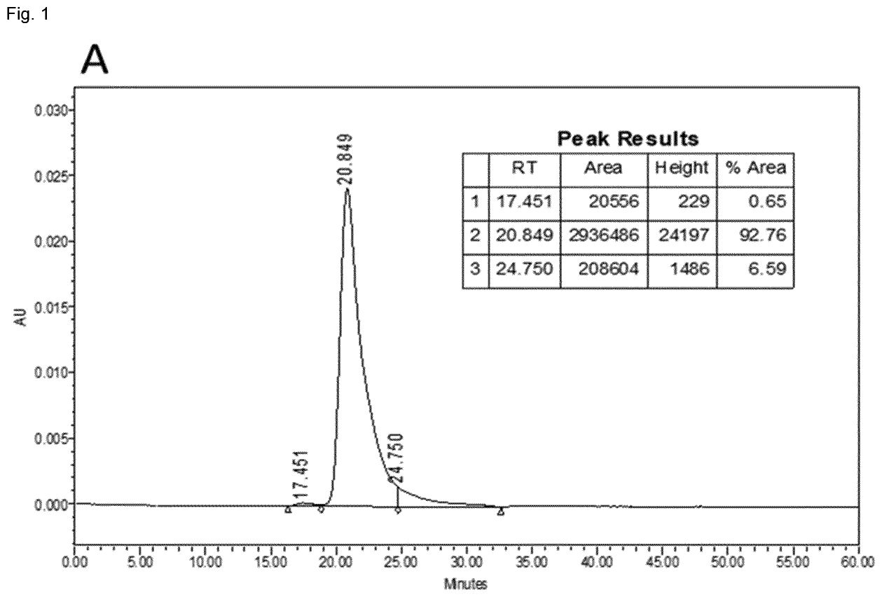

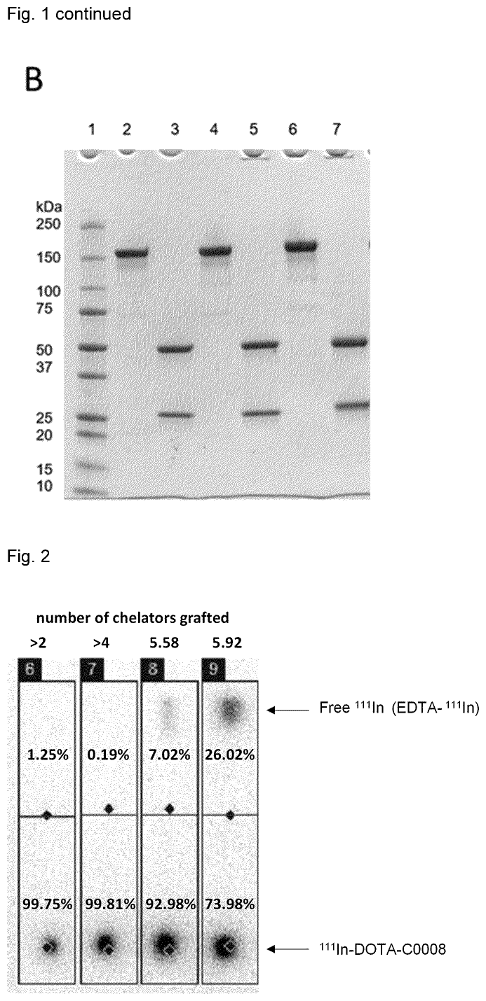

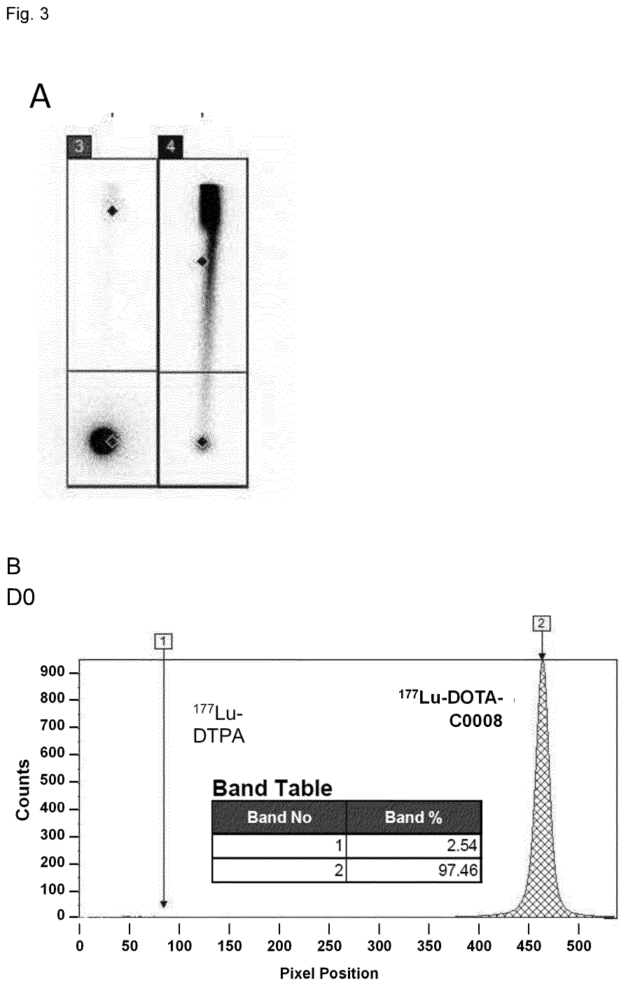

Results: Incubation of antibody C0008 with SCN-Bn-DOTA and subsequent purification yielded a DOTA-C0008 conjugate of high chemical purity, as shown by SE-HPLC (FIG. 1A) and SDS PAGE (FIG. 1B). The resultant DOTA-C0008 conjugate was analyzed for the number of DOTA molecules attached per antibody and results from the ITLC are shown in FIG. 2. An average of about 5.8 DOTA molecules per antibody was obtained under the chosen conditions. DOTA-C0008 conjugate was then labelled successfully with lutetium-177 and the resultant radioimmunoconjugate showed a specific activity of 16 mCi/mg (592 MBq/mg) with an overall radiochemical purity of 97.5%, as determined by ITLC (FIG. 3A) and Autoradiography (FIG. 3B).

Conclusion: An efficient method to radiolabel the anti-oxMIF antibody C0008 with 177Lu was elaborated. The resultant radioimmunoconjugate showed sufficient purity and specific activity.

FIG. 1 shows the purity and integrity analysis of DOTA-C0008; A: DOTA-C0008 SE-HPLC profile (2=280 nm). B: Coomassie stained SDS PAGE: 1: molecular weight standard; 2: C0008 non-reduced; 3: C0008 reduced; 4: concentrated C0008 (labeling batch) non-reduced; 5: concentrated C0008 (labeling batch) reduced; 6: DOTA-C0008 non-reduced; 7: DOTA-C0008 reduced.

FIG. 2 shows the determination of the number of DOTA molecules grafted to-C0008 determined by ITLC: ITLC separation of DOTA-C0008 which was incubated with a mixture of tracer amounts of radioactive indium (¹¹¹In) and stable indium (¹¹⁵In) at 2 (lane 6), 4 (lane 7), 6 (lane 8) or 8 (lane 9) fold excess of ^{111/115}In in proportion to the amount of DOTA conjugated antibody.

FIG. 3 shows the analysis of radiolabeled C0008 by ITLC and Autoradiography: A: ITLC of radiolabeled C0008 (Lane 3: ¹⁷⁷Lu labelling reaction with DOTA-C0008; Lane 4: ¹⁷⁷Lu labelling reaction with C0008); B: Autoradiogram of ITLC film lane 3 (¹⁷⁷Lu-DOTA-C0008 after DTPA addition; Peak 1 reflects free ¹⁷⁷Lu-DTPA and Peak 2 reflects ¹⁷⁷Lu-DOTA-C0008).

### Example 2

Stability of 177Lu-DOTA-C0008 in formulation buffer

### Materials and Methods

A solution of 177Lu-DOTA-C0008 was prepared to achieve a final concentration of 700 μg/mL suitable for stability studies. The characteristics of the ¹⁷⁷Lu-DOTA-C0008 solution are presented in Table 2.

TABLE 2
Characteristics of ¹⁷⁷Lu-DOTA-C0008 solution

| Dosing solution | ¹⁷⁷Lu-DOTA-C0008 |
|---|---|
| Specific Activity | 15.97 mCi/mg (590.89 MBq/mg) |
| Radioactive concentration | 10.86 mCi/mL |
| Concentration mAb | 0.66 mg/mL in formulation buffer |
| Final Radiopurity ITLC | 97.40% |
| Buffer | 0.03M phosphate buffer |
| | 0.015 g/mL Sodium Ascorbate |
| | 0.015 mg/mL BSA |
| | 0.19 mM DTPA |

¹⁷⁷Lu-DOTA-C0008 in formulation buffer stored at 4° C. was analyzed by ITLC and SE-HPLC at different time points. Radiochemical purity was determined by ITLC eluent with citrate 0.1 M pH 5, in order to quantify the percentage of radioactivity release from the radioimmunoconjugate over time. SE-HPLC was conducted on a Superdex 200, 10/300 GL column eluted with phosphate buffer containing arginine at a flow rate of 0.6 mL/min to determine aggregation and contaminants (i.e. ¹⁷⁷Lu-DTPA or ¹⁷⁷Lu-DOTA). The chromatographic profiles were acquired with a Radiomatic 150TR flow scintillation analyser.

In addition, the radioactivity of the radioimmunoconjugate was measured by gamma counting to give information about the solubility of ¹⁷⁷Lu-DOTA-C0008 in formulation buffer. For this purpose, 5 μL were counted directly from 1/20 dilution in formulation buffer.

Results: ITLC analysis of the 177Lu-DOTA-C0008 solution revealed almost no release of radioactivity from the radioimmunoconjugate over time. After 7 days, the solution of radiolabeled antibody presented a radiopurity up to 96.25%. The radiolabeled antibody released about 1.2% of the initial bound radioactivity (Table 2) when kept at 4° C. in formulation buffer (Table 3). The 177Lu-DOTA-C0008 eluted at 21.6 min and its contaminants (¹⁷⁷Lu-DTPA and ¹⁷⁷Lu-DOTA derivatives) eluted at 32.8 min. There was a slight increase for HMW aggregates from day 0 to day 3, but no additional aggregates were detected after day 3 (Table 5). After 7 days, the dosing solution presented 93% intact antibody by SE-HPLC. The SE-HPLC results were in accordance with ITLC results. The radioactivity monitored over time was in good agreement with the theoretical radioactive decay for lutetium-177 (values in brackets in Table 4).

Conclusion: ¹⁷⁷Lu-DOTA-C0008 was stable in formulation buffer up to 7 days when tested with ITLC and SE-HPLC.

TABLE 3
ITLC - Radiopurity of ¹⁷⁷Lu-DOTA-C0008
in formulation buffer at 4° C.

| Time point | ¹⁷⁷Lu-DOTA-C0008 %* |
|---|---|
| D 0 | 97.40% |
| D 1 | 97.35% |
| D 2 | 96.81% |
| D 3 | 96.54% |
| D 7 | 96.25% |

*percentage of antibody-associated ¹⁷⁷Lu, corresponding to intact protein, protein fragments and aggregates

TABLE 4
Radioactive concentration of ¹⁷⁷Lu-DOTA-C0008
in formulation buffer at 4° C.

| Time point | Radioactivity |
|---|---|
| D 0 | 10.86 mCi/mL |
| D 3 | 7.86 mCi/mL (7.8 mCi/mL) |
| D 7 | 5.00 mCi/mL (5.2 mCi/mL) |

TABLE 5
Stability of ¹⁷⁷Lu-DOTA-C0008 in formulation buffer (SE-HPLC)

| Time point | SE-HPLC results |
|---|---|
| D 0 | 95.15% (intact Ab) |
| | 0.93% HMW |
| | 3.92% LMW |
| D 3 | 92.67% (intact Ab) |
| | 2.21% HMW |
| | 5.12% LMW |
| D 7 | 93.10% (intact Ab) |
| | 2.53% HMW |
| | 4.14% LMW |

HMW: high molecular weight impurities as aggregates and oligomers,
LMW: low molecular weight impurities as ¹⁷⁷Lu-DTPA or ¹⁷⁷Lu-DOTA derivatives

### Example 3

Stability of 177Lu-DOTA-C0008 in mouse plasma

Materials and Methods: Plasma stability of ¹⁷⁷Lu-DOTA-C0008 was assessed using fresh mouse plasma (prepared with heparin lithium tubes) at 37° C. The concentration of ¹⁷⁷Lu-DOTA-C0008 in plasma was 70 μg/mL. The stability in plasma was evaluated at day 0, day 1, day 2, day 3 and day 7 by ITLC (eluting in citrate 0.1 M pH 5), autoradiography and SDS-PAGE.

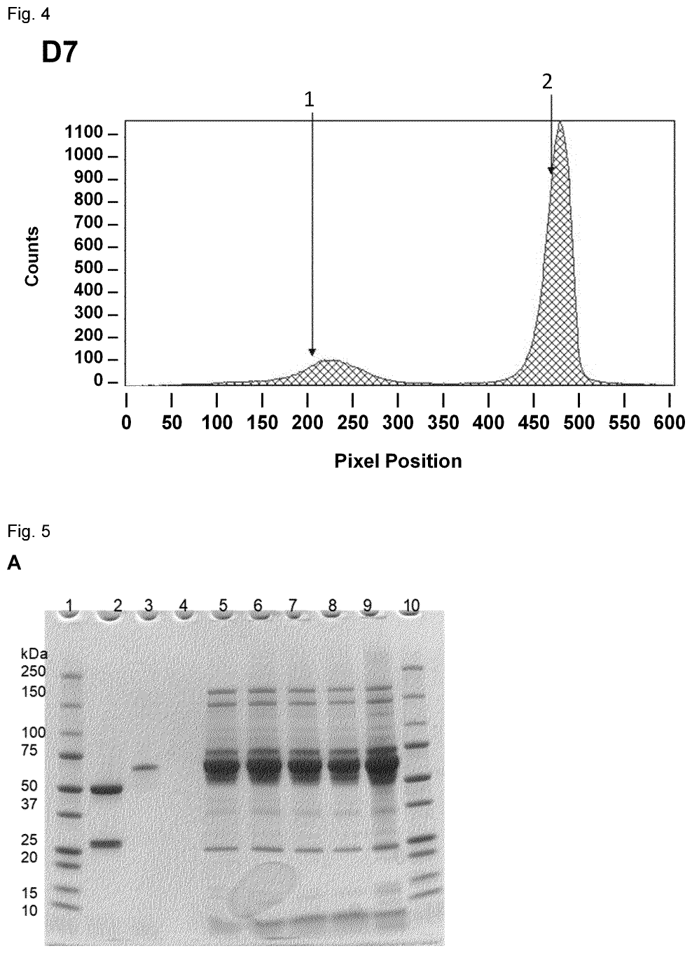

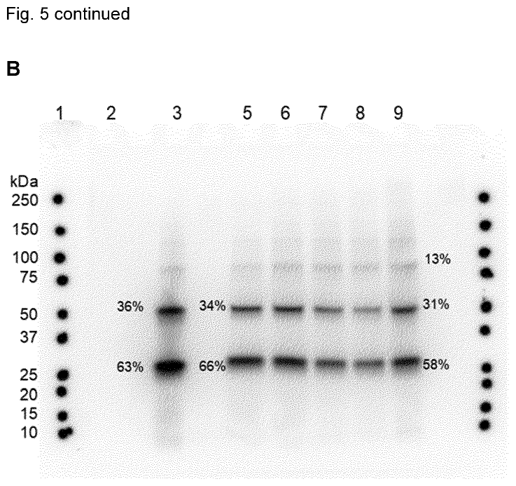

Results: After 7 days of incubation in mouse plasma at 37° C., the solution of radiolabeled antibody presented a radiopurity of about 80% with a corresponding release of about 20% of the initial bound radioactivity (Table 6). The released radioactivity corresponded to ¹⁷⁷Lu-DOTA-C0008 derivatives and free ¹⁷⁷Lu as demonstrated by ITLC results (FIG. 4). SDS-PAGE analyses (FIG. 5) revealed no significant degradation of the radiolabeled antibody when incubated in plasma. In addition, no trans-chelation of ¹⁷⁷Lu to other plasma proteins was observed (FIG. 5).

Conclusion: The stability results in plasma was acceptable with about 20% ¹⁷⁷Lu activity released after 7 days at 37° C. Minor, small radioactive products observed during the stability study most likely correspond to ¹⁷⁷Lu-DOTA-C0008 derivatives and free ¹⁷⁷Lu. As shown below, the reduction in hydrophobicity of the newly designed molecule C0083 resulted in greatly enhanced plasma stability of the resulting ¹⁷⁷Lu-DOTA-C0083 radio-immunoconjugate compared to ¹⁷⁷Lu-DOTA-C0008.

TABLE 6
Stability of ¹⁷⁷Lu-DOTA-C0008 in mouse plasma (ITLC)

| Time point | ¹⁷⁷Lu-DOTA-C0008 % |
|---|---|
| D 0 | 97.65% |
| D 1 | 87.20% |
| D 2 | 83.94% |
| D 3 | 82.18% |
| D 7 | 79.06% |

FIG. 4 shows the plasma stability of ¹⁷⁷Lu-DOTA-C0008 (autoradiography): Autoradiogram of ¹⁷⁷Lu-DOTA-C0008 incubated in mouse plasma for 7 days; 1: ¹⁷⁷Lu-DOTA-C0008 derivatives and free ¹⁷⁷Lu; 2: ¹⁷⁷Lu-DOTA-C0008.

FIG. 5 shows the plasma stability of ¹⁷⁷Lu-DOTA-C0008 (SDS-PAGE): Reduced SDS PAGE with ¹⁷⁷Lu-DOTA-C0008 in mouse plasma (left: Coomassie staining; right: Autoradiography). Lane 1 and Lane 10: molecular weight standards; Lane 2: DOTA-C0008; Lane 3: ¹⁷⁷Lu-DOTA-C0008 in buffer (TO); Lane 5: ¹⁷⁷Lu-DOTA-C0008 (TO hours in plasma); Lane 6: ¹⁷⁷Lu-DOTA-C0008 (1 day in plasma), Lane 7: ¹⁷⁷Lu-DOTA-C0008 (2 days in plasma); Lane 8: ¹⁷⁷Lu-DOTA-C0008 (3 days in plasma); Lane 9: ¹⁷⁷Lu-DOTA-C0008 (7 days in plasma).

### Example 4

Biodistribution of C0008 in CT26 tumor bearing Balb/c mice

Materials and Methods: Biodistribution of C0008 was investigated in female Balb/c mice carrying subcutaneous tumors of the syngeneic colon cancer cell line CT26. 10 female Balb/c mice received unilateral, subcutaneous injections of 3×10⁵ CT26 cells in PBS (100 μl/animal). Upon reaching individual tumor volumes of 150-300 mm³, mice received a single intravenous dose of 5 mg/kg IRDye 800CW-labelled C0008. Two mice were used as untreated ‘no signal’ control.

C0008 was labelled with IRDye 800CW using the IRDye 800CW Protein labeling kit—high MW from LI-COR Biosciences following the manufacturer's instructions. After the labelling process and prior to injection of labeled C0008 into mice, the protein concentration and labeling efficiency of the IRDye 800CW labeled antibody was determined using the Nanodrop technology, and mice were dosed based on the protein concentration after labelling. In vivo imaging was performed in a LI-COR Pearl® Trilogy imaging device upon administration of labelled C0008 at the following time-points: 1 h, 6-8 h, 24 h, 48 h, 72 h, 96 h, 168 h after dosing. Image analysis was performed to quantify the relative fluorescence intensity (RFU) of the antibody in the tumor (RFU=RFU tumor area−RFU background/mm²*tumor area).

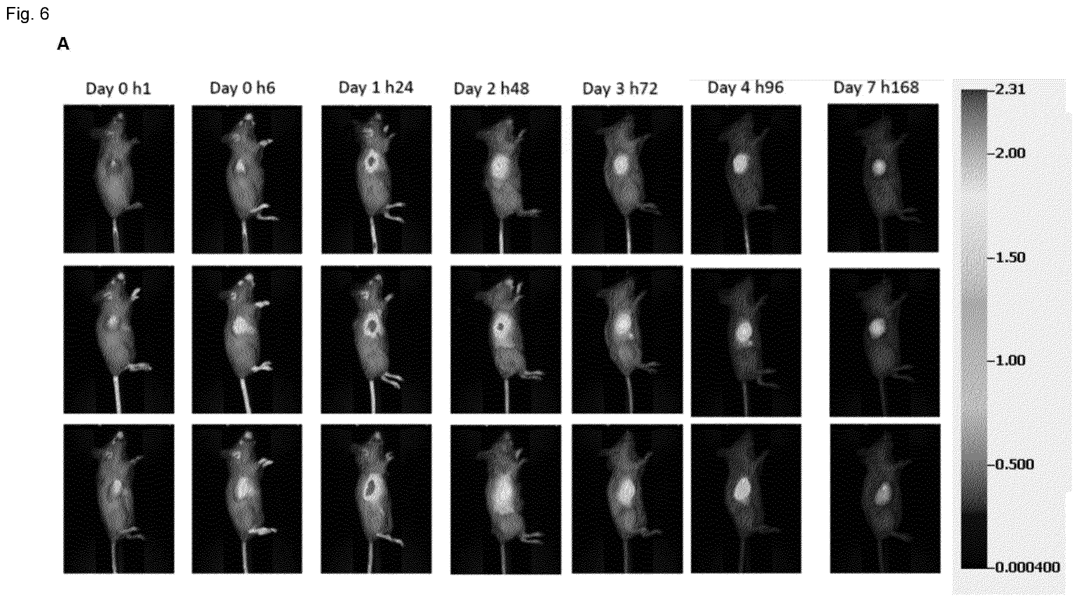

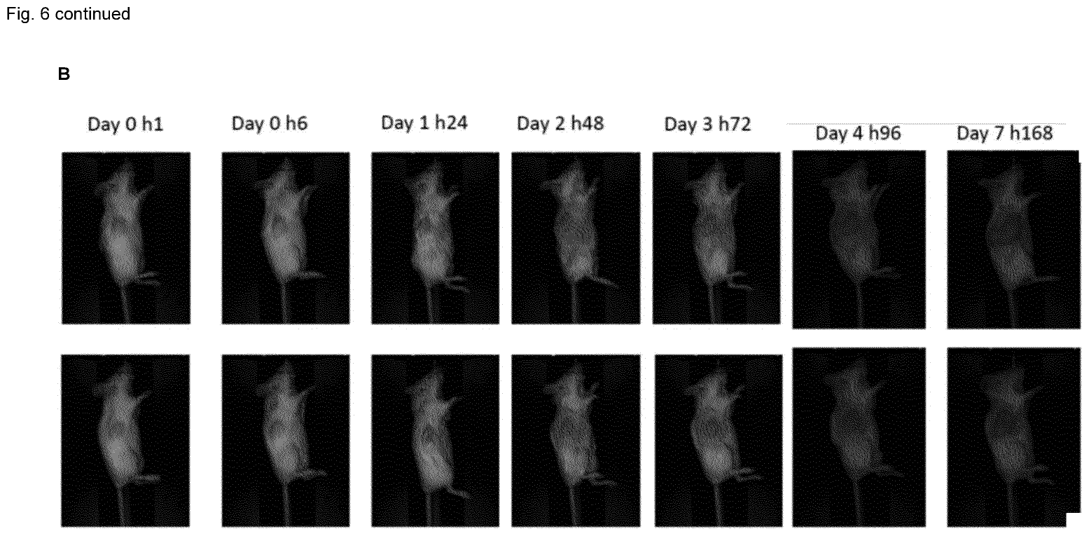

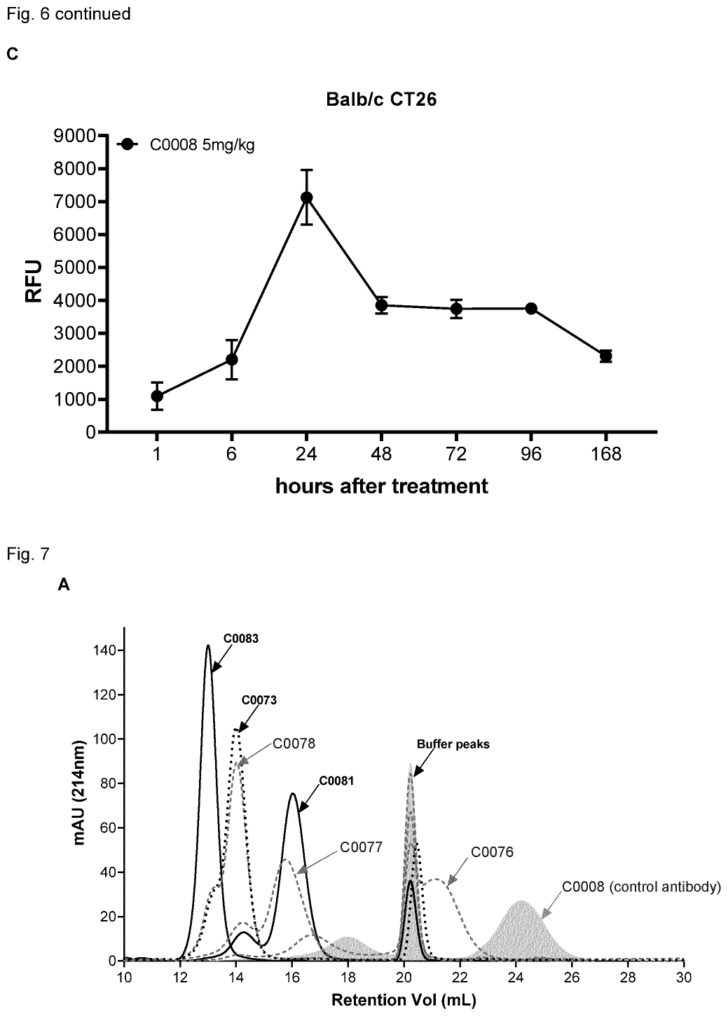

Results: FIG. 6A shows a significant intra-tumoral distribution of intravenously administered IRDye 800CW-labeled C0008 with a peak at 24 h and tumor retention up to 7 days. No signal was detected in untreated control mice (FIG. 6B).

Conclusion: The biodistribution study suggests the suitability of oxMIF antibodies as radiopharmaceuticals.

FIG. 6 shows the biodistribution of IRDye 800CW labeled C0008 in female Balb/c carrying subcutaneous CT26 tumors: Infra-red in vivo imaging of mice carrying subcutaneous CT26 tumors. Infra-red images of mice were taken 1 h, 6 h, 24 h, 48 h, 72 h, 96 h and 168 h post injection of A: IRDye 800CW-labeled C0008 (5 mg/kg) or B: without treatment (scale bar applies to A and B); C: tumor penetration and retention of C0008 quantified by digital image analysis.

### Example 5

TABLE 7
Variant IDs and sequence combinations of the
antibodies used in the radioimmunoconjugates

| Variant ID | | | | |
|---|---|---|---|---|
| (Sample) | VH | CH | VL | CL |
| C0008 | wt | wt | wt | SEQ ID NO: 41 |
| (Imalumab | (SEQ ID NO: 29) | (SEQ ID NO: 37) | (SEQ ID NO: 32) | |
| control | | | | |
| antibody) | | | | |
| C0113 | L5Q/W97Y | L234A/L235A/I253A | F49Y/A51G/W93F | SEQ ID NO: 41 |
| | (SEQ ID NO: 31) | (SEQ ID NO: 39) | (SEQ ID NO: 34) | |
| C0114 | L5Q/W97Y | L234A/L235A/ | F49Y/A51G/W93F | SEQ ID NO: 41 |
| | (SEQ ID NO: 31) | H310A/H435Q | (SEQ ID NO: 34) | |
| | | (SEQ ID NO: 40) | | |
| C0115 | L5Q/W97Y | L234A/L235A | F49Y/A51G/W93F | SEQ ID NO: 41 |
| | (SEQ ID NO: 31) | (SEQ ID NO: 38) | (SEQ ID NO: 34) | |
| C0116 | L5Q/W97Y | L234A/L235A/I253A | M30L/F49Y/A51G/ | SEQ ID NO: 41 |
| | (SEQ ID NO: 31) | (SEQ ID NO: 39) | P80S/W93F | |
| | | | (SEQ ID NO: 36) | |
| C0117 | L5Q/W97Y | L234A/L235A/ | M30L/F49Y/A51G/ | SEQ ID NO: 41 |
| | (SEQ ID NO: 31) | H310A/H435Q | P80S/W93F | |
| | | (SEQ ID NO: 40) | (SEQ ID NO: 36) | |
| C0118 | L5Q/W97Y | L234A/L235A | M30L/F49Y/A51G/ | SEQ ID NO: 41 |
| | (SEQ ID NO: 31) | (SEQ ID NO: 38) | P80S/W93F | |
| | | | (SEQ ID NO: 36) | |
| C0073 | wt | wt | F49Y/A51G/W93F | SEQ ID NO: 41 |
| | (SEQ ID NO: 29) | (SEQ ID NO: 37) | (SEQ ID NO: 34) | |
| C0076 | L5Q | wt | F49Y/A51G | SEQ ID NO: 41 |
| | (SEQ ID NO: 30) | (SEQ ID NO: 37) | (SEQ ID NO: 33) | |
| C0077 | L5Q | wt | Y36F/F49Y/A51G | SEQ ID NO: 41 |
| | (SEQ ID NO: 30) | (SEQ ID NO: 37) | (SEQ ID NO: 33 | |
| | | | with. Y36F) | |
| C0078 | L5Q | wt | F49Y/A51G/W93F | SEQ ID NO: 41 |
| | (SEQ ID NO: 30) | (SEQ ID NO: 37) | (SEQ ID NO: 34) | |
| C0081 | L5Q/W97Y | wt | F49Y/A51G | SEQ ID NO: 41 |
| | (SEQ ID NO: 31) | (SEQ ID NO: 37) | (SEQ ID NO: 33) | |
| C0083 | L5Q/W97Y | wt | F49Y/A51G/W93F | SEQ ID NO: 41 |
| | (SEQ ID NO: 31) | (SEQ ID NO: 37) | (SEQ ID NO: 34) | |
| C0090 | L5Q/W97Y | wt | F49Y/A51G/M30L/ | SEQ ID NO: 41 |
| | (SEQ ID NO: 31) | (SEQ ID NO: 37) | W93F/P80S | |
| | | | (SEQ ID NO: 36) | |

Reduced aggregation propensity and reduced hydrophobicity of newly designed anti-oxMIF antibodies.

Materials and Methods: To evaluate hydrophobicity and aggregation the newly 5 designed antibodies were analyzed by gel filtration (SEC) using two different SEC columns and running buffer conditions and by hydrophobic interaction chromatography (HIC) and compared to anti-oxMIF antibody C0008.

For SEC, samples were diluted to 0.1 mg/ml in 5x phosphate buffered saline including 0.02% Tween (5×PBST) and 50 μl sample was applied to a Superdex 200 increase 10/300 GL (GE Healthcare) gel-filtration column at a flow rate of 0.75 ml/min. Separations and equilibration were performed in 5×PBST at 22° C. Protein peaks were monitored using absorbance at 214 nm and spectra were analyzed using the Unicorn emulation software package (GE Healthcare). Additionally, samples were diluted to 0.2 mg/ml in 1x phosphate buffered saline (1xPBS) and 100 μl sample was applied to an Enrich 650 (Bio-Rad) gel-filtration column at a flow rate of 1 ml/min. Separations and equilibration were performed in 1xPBS at 22° C. Protein peaks were monitored using absorbance at 280 nm and spectra were analyzed using the ChromLab software package (Bio-Rad). Results are reported in retention volume (Vr, ml) for the main peak and presence of aggregates was ranked manually.

For HIC analysis, all samples were diluted to a final concentration of 0.2 mg/ml using 50 mM phosphate and 0.75 M Ammonium Sulphate, pH 6.9. Highly purified samples of antibodies were loaded independently onto a 1 ml HiTrap Butyl HP column. 50 μl samples were injected and the column flow rate was maintained at 1 ml/min at 22° C. Separation of peaks was carried out over a 20-column volume (CV) gradient from 0-100% B (Buffer B: 50 mM phosphate, 20% isopropanol; pH7.0). Protein peaks were monitored using absorbance at 280 nm and spectra were analyzed using the Unicorn emulation software package (GE healthcare). Results are reported in Vr (ml) at peak maximum, for each peak.

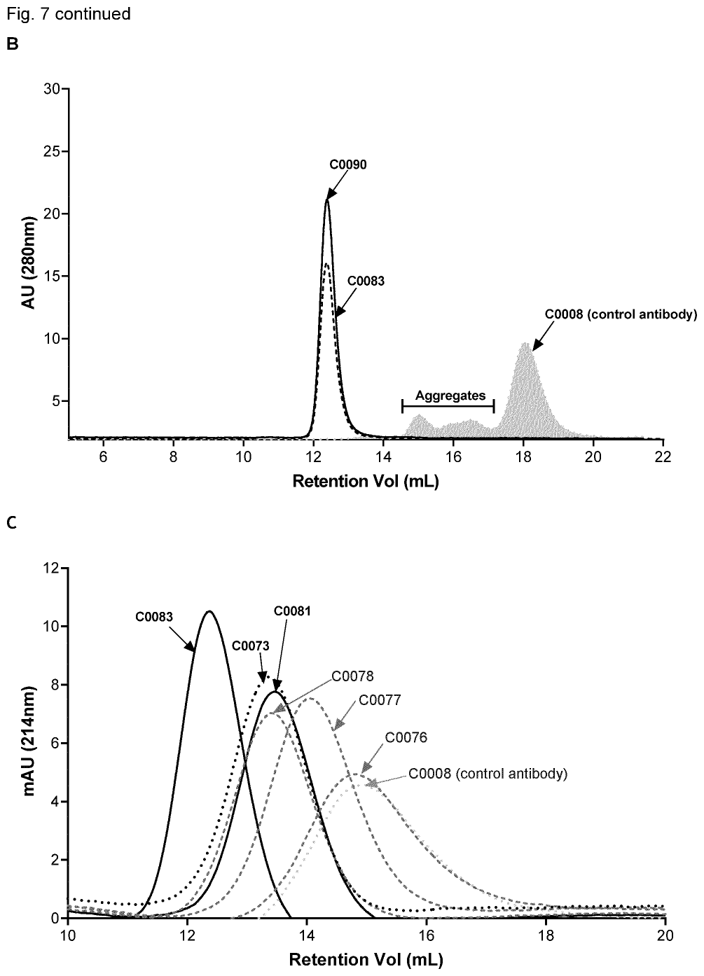

Results: Control antibody C0008 demonstrated a retention volume close to the bed volume of the size exclusion columns (˜24 ml Superdex 200, ˜18 ml Enrich 650), which corresponds to a molecular weight far smaller than expected for a human IgG (FIGS. 7A and B). The unusual long retention was mainly due to hydrophobic interactions with the stationary phase surface. In addition to the high retention volume, significant amounts of IgG dimers and aggregates were present for C0008. All newly designed antibodies showed a reduced retention volume (Vr) demonstrating reduced interaction with the column and thus reduced hydrophobicity of the molecules (Table 8, FIGS. 7A and B). C0083 and C0090 showed the lowest retention volume (Table 8, FIG. 7B) and the Vr corresponded to the molecular weight of a monomeric human IgG, when compared to a molecular weight standard. Additionally, antibody dimers and aggregation were significantly reduced in samples of newly designed antibodies C0073, C0078, C0083 and C0090 (FIGS. 7A and B). Especially C0083 and C0090 showed a single monomer peak only.

Using the HIC column retention volumes, antibodies were ranked from the least to the most (lowest to highest retention volume) hydrophobic IgG (FIG. 7C, Table 9), and the results confirmed the data from SEC analysis. The newly designed antibody C0083 (retention volume of 12.37) was the least hydrophobic, whereas the control antibody C0008 (retention volume of 14.92 ml) showed the highest hydrophobicity.

Conclusion: It is evident from SEC and HIC analysis that the optimization resulted in improved anti-oxMIF antibodies with reduced hydrophobicity and aggregation propensity.

TABLE 8
Size Exclusion Chromatography

| Sample | Running Buffer | Column | Vr (ml) | Aggregates |
|---|---|---|---|---|
| C0008 | 5xPBS | Superdex 200 | 24.20 | ++ |
| C0073 | 5xPBS | Superdex 200 | 14.00 | (+) |
| C0076 | 5xPBS | Superdex 200 | 21.10 | + |
| C0077 | 5xPBS | Superdex 200 | 15.77 | + |
| C0078 | 5xPBS | Superdex 200 | 14.02 | (+) |
| C0081 | 5xPBS | Superdex 200 | 16.03 | + |
| C0083 | 5xPBS | Superdex 200 | 13.01 | − |
| C0008 | 1xPBS | Enrich 650 | 18.06 | + |
| C0083 | 1xPBS | Enrich 650 | 12.39 | − |
| C0090 | 1xPBS | Enrich 650 | 12.38 | − |

TABLE 9
Hydrophobic Interaction Chromatography

| Sample | Vr (ml) |
|---|---|
| C0008 | 14.92 |
| C0073 | 13.37 |
| C0076 | 14.84 |
| C0077 | 14.05 |
| C0078 | 13.41 |
| C0081 | 13.48 |
| C0083 | 12.37 |

FIG. 7 shows the chromatography profiles demonstrating reduced aggregation and hydrophobicity of the newly designed anti-oxMIF antibodies. (A) Comparison of elution profiles of C0008 (control antibody, gray area) and newly designed antibodies on a Superdex 200 increase 10/300 GL gel filtration column using 5×PBST as mobile phase. (B) Comparison of elution profiles of C0008 (control antibody, gray area) and newly designed antibodies on a Enrich 650 gel filtration column using 1xPBS as mobile phase (C) Comparison of elution profiles of C0008 (control antibody, gray area) and newly designed antibodies on a HiTrap Butyl HP HIC column.

### Example 6

Binding of newly designed anti-oxMIF antibodies to immobilized MIF (KD determination)

Material and Methods: Recombinant human MIF diluted in PBS at 1 μg/ml was immobilized into ELISA plates overnight at 4° C. (transforming MIF to oxMIF according to Thiele et al., 2015). After blocking, serial dilutions of anti-oxMIF antibodies were added to the plates. Finally, bound antibodies were detected using a protein L-HRP conjugate and tetramethylbenzidine (TMB) as substrate. The chromogenic reaction was stopped with 3M H₂SO₄ and OD was measured at 450 nm. Data from different experiments were normalized to the maximal OD of the anti-oxMIF antibody C0008 (=100%) of the respective experiment and EC₅₀ values were determined by 4-parameter fit using GraphPad Prism (and the mean of two experiments is shown).

Results and conclusion: The binding of the newly designed antibodies towards immobilized MIF (oxMIF) was measured over a broad range of concentrations and the resulting binding curves are illustrated in FIG. 8. The anti-oxMIF antibody C0008 was used as reference for oxMIF binding. The binding curves and the calculated EC₅₀ values, representing the KD, clearly showed that the newly designed antibodies C0073, C0076, C0078, C0083 and C0090 retained their affinity (EC₅₀ range 0.5-1.6 nM, which is within the assay variability) to oxMIF, compared to C0008 (EC₅₀ 0.7 nM) (FIG. 3A). C0077 showed a 5-fold reduction in affinity to oxMIF due to point mutation (Y36F) in VL compared to C0076 (FIG. 8), although hydrophobicity of C0077 was markedly reduced (FIG. 7A).

FIG. 8 shows the binding curves of newly designed anti-oxMIF antibodies to immobilized MIF (KD determination). C0008 was used as reference antibody.

TABLE 10
K_{D} values of anti-oxMIF antibodies

| Sample | K_{D} (nM) |
|---|---|
| C0008 | 0.7 |
| C0073 | 1.0 |
| C0076 | 1.1 |
| C0077 | 5.2 |
| C0078 | 1.6 |
| C0083 | 0.5 |
| C0090 | 0.5 |

### Example 7

Differential binding of newly designed anti-oxMIF antibodies to oxMIF vs. redMIF.

Material and Methods: Anti-oxMIF antibodies were immobilized into microplates over night at 4° C. at a concentration of 15 nM. After blocking, wells were incubated with 50 ng/ml of either redMIF or the oxMIF surrogate NTB-MIF (Schinagl et al., 2018). Captured oxMIF was detected with a biotinylated polyclonal rabbit anti-MIF antibody and Streptavidin-HRP conjugate. Plates were stained with tetramethylbenzidine (TMB) and chromogenic reaction was stopped by addition of 30% H₂SO₄. OD was measured at 450 nm. Data from different experiments were normalized to the maximal OD of the anti-oxMIF antibody C0008 (=100%) of the respective experiment, and the mean of two to three experiments is shown.

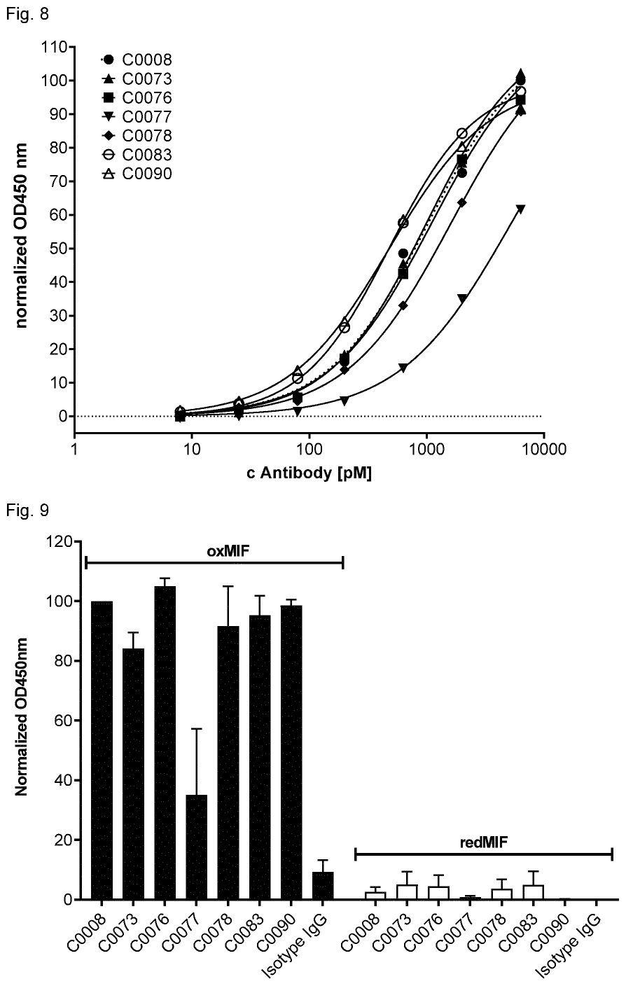

Results and conclusion: The binding of the newly designed antibodies towards soluble oxMIF and redMIF are illustrated in FIG. 9. The results clearly showed that the newly designed antibodies C0073, C0076, C0078, C0083 and C0090 demonstrated strong binding to oxMIF but no binding to redMIF, thus the antibodies with reduced hydrophobicity and aggregation retained their ability to discriminate between oxMIF and redMIF. The newly designed antibodies showed similar ODs compared to the antibody C0008, which has been described to discriminate between oxMIF and redMIF (Thiele et al., 2015). (FIG. 9). C0077 showed that binding to oxMIF was significantly reduced due to point mutation (Y36F) in VL compared to C0076 (FIG. 9). C0077 shows markedly reduced hydrophobicity (FIG. 7A) but failed to maintain the binding properties.

FIG. 9 shows the differential binding of the newly designed antibodies to oxMIF vs. redMIF. C0008 was used as reference antibody.

### Example 8

Improved production of newly designed antibody C0083.

To compare the expression yields, the newly designed anti-oxMIF antibody C0083 and C0008 were produced by transient expression in ExpiCHO cells (Thermo Fisher). Briefly, 50-200 ml of exponentially growing ExpiCHO cells were transiently transfected using ExpiFectamine CHO Kit (Thermo Scientific) and were cultured for 8 days according to the manufacturer's instructions “standard protocol” (Thermo Scientific) at 37° C. in a humidified incubator. Cells were removed by centrifugation and the supernatants were diluted 1:1 with 40 mM sodium phosphate, 300 mM sodium chloride, pH 7.2. Diluted supernatants were 0.2 μm filtered and were applied to a Mab Select Prism A column (Cytiva) using an NGC QUEST 10 Chromatography System (Bio-Rad) at room temperature. The column was washed with 10 column volumes of 20 mM sodium phosphate, 150 mM sodium chloride, pH 7.2 and antibodies were eluted in 10 column volumes of 100 mM glycine, pH 3.5. Antibodies were immediately neutralized to pH 5 and formulated in 1xPBS pH 7.2 by Bio-Scale P-6 desalting cartridges and were concentrated to ˜1 mg/ml using Amicon Ultra-15 centrifugal devices (Merck Millipore). Protein concentration was determined using NanoDrop technology and their respective extinction coefficients. The amount of purified antibody after Mab Select Prism A and neutralization (final yield of the respective antibody) was divided by the cell culture volume and the resultant value is herein referred to as antibody titer (mg/L).

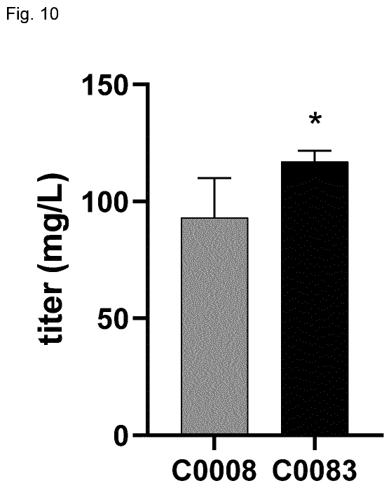

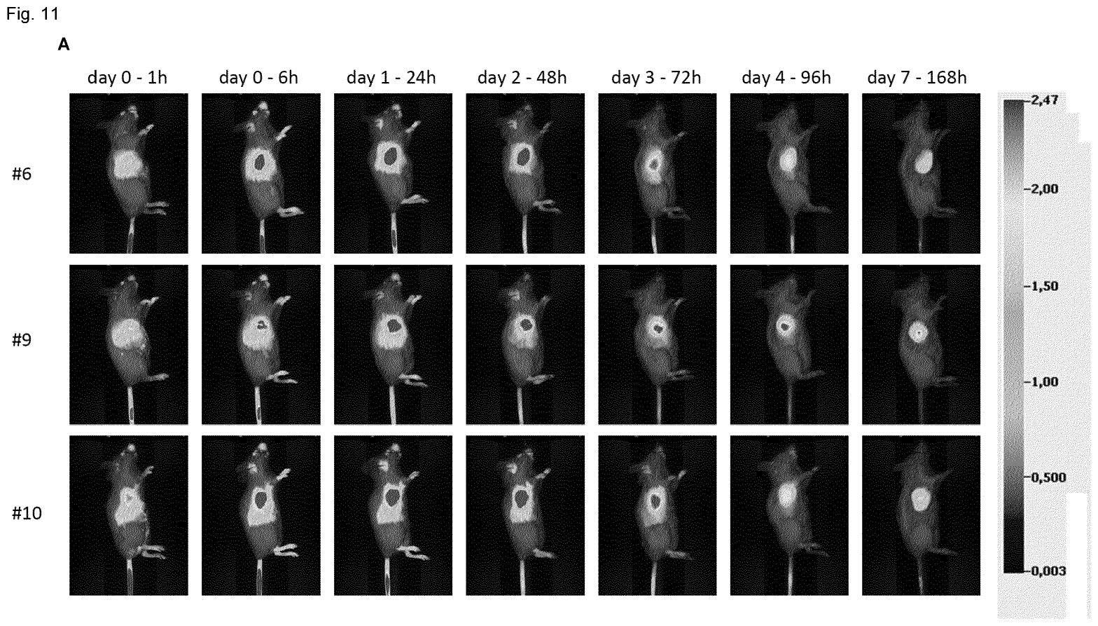

Results: 3 individual productions of C0083 in comparison to C0008 clearly showed that the newly designed antibody C0083 was produced significantly (unpaired t-test, p=0.03) at higher levels (FIG. 10).

Conclusion: Significantly higher antibody titers were obtained after optimizing the antibody regarding reduced hydrophobicity and aggregation propensity compared to C0008 (117 mg/L C0083 vs. 93 mg/ml C0008).

FIG. 10 shows the improved production of the newly designed anti-oxMIF antibody C0083 vs. control antibody C0008.

### Example 9

Biodistribution of newly designed anti-oxMIF antibody C0083 in CT26 tumor bearing Balb/c mice

Materials and Methods: Biodistribution of newly designed anti-oxMIF antibody C0083 was investigated in female Balb/c mice carrying subcutaneous tumors of the syngeneic colon cancer model CT26. Female Balb/c mice received unilateral, subcutaneous injections of 3×10⁵ CT26 cells in PBS (100 μl/animal). Upon reaching individual tumor volumes of 150-300 mm³, mice were assigned to treatment groups and received a single intravenous dose of 5 mg/kg IRDye 800CW-labeled C0083.

C0083 was labelled with IRDye 800CW using the IRDye 800CW Protein labeling kit-high MW from LI-COR Biosciences following the manufacturer's instructions. After the labelling process and prior to injection of labeled antibodies into mice, the protein concentration and labeling efficiency of the IRDye 800CW labeled antibody was determined using the Nanodrop technology, and mice were dosed based on the protein concentration after labelling. In vivo imaging was performed in a LI-COR Pearl® Trilogy imaging device upon administration of labelled antibodies at the following time-points: 1 h, 6-8 h, 24 h, 48 h, 72 h, 96 h, 168 h after dosing. Image analysis was performed to quantify the relative fluorescence intensity (RFU) of the antibody in the tumor (RFU=RFU tumor area-RFU background/mm²*tumor area)

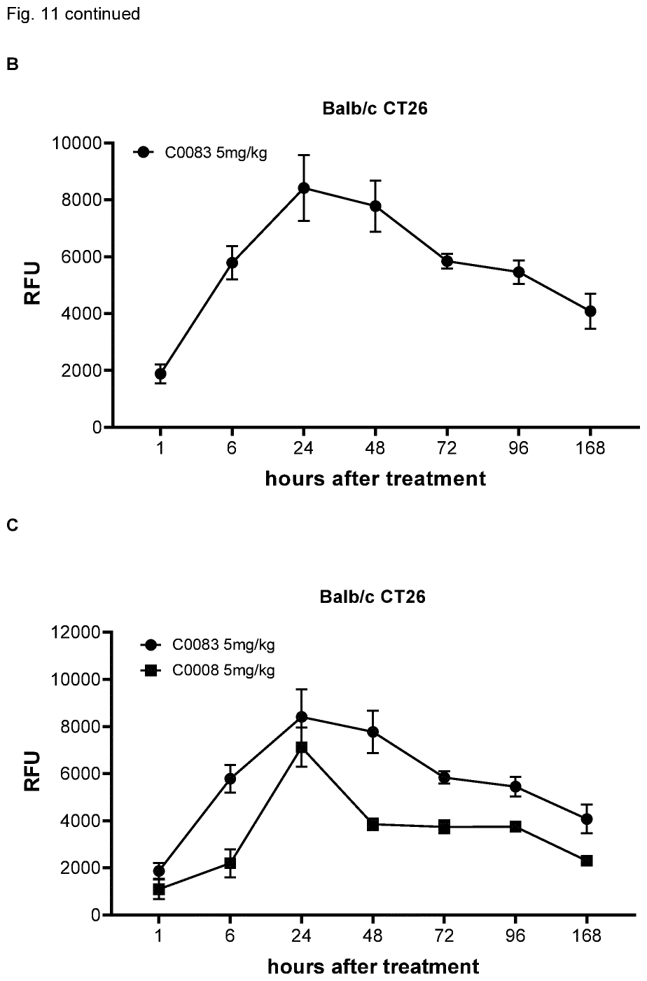

Results: A significant intra-tumoral distribution of intravenously administered IRDye 800CW-labeled C0083 (FIG. 11A-B) with a peak at 24 h and tumor retention of more than 7 days was determined.

Conclusion: The results clearly demonstrate that C0083 has improved tumor penetration and retention properties compared to C0008 (FIG. 6A/FIG. 11C). Additionally, the biodistribution study suggests the suitability of C0083 for application as radioimmunoconjugate.

FIG. 11 shows the tumor penetration of newly designed anti-oxMIF antibody C0083 vs. C0008 by infra-red in vivo imaging of mice carrying subcutaneous CT26 tumors. A: Infra-red images of mice were taken 1 h, 6 h, 24 h, 48 h, 72 h, 96 h and 168 h post injection of the IRDye 800CW labeled C0083 dosed at 5 mg/kg; B: tumor penetration and retention of C0083 quantified by digital image analysis. C: Comparison of tumor penetration and retention of IRDye 800CW labeled C0083 vs. C0008 quantified by digital image analysis

### Example 10

Generation of a 177Lu-Radioimmunoconjugate of the newly designed C0083

### Materials and Methods

C0083 was incubated with SCN-Bn-DOTA at a molar ratio of 1:10 in HEPES 0.06 M pH 9. The labelling mixture was stirred for 2 hours at 37° C. After the reaction, excess SCN-Bn-DOTA was removed by multiple centrifugations in 0.9% NaCl at 3000 g using 30 kDa MWCO Amicon Ultra centrifugal filters (regenerated cellulose, Millipore). The conjugate was examined by SE-HPLC to evaluate the chemical purity at the end of the purification. SE-HPLC was conducted on a Superdex 200 10/300 GL column eluted with phosphate buffer containing arginine at a flow rate of 0.6 mL/min. In addition, the purity and integrity of DOTA-C0083 was assessed by SDS-PAGE under reducing and non-reducing conditions (4-15% linear gradient polyacrylamide gel; Bio-Rad Laboratories).

The number of attached DOTA molecules per antibody was determined by using the method of Meares et al (1984) and Instant Thin-Layer Chromatography (ITLC). A small aliquot of DOTA conjugated antibody was incubated with a mixture of tracer amounts of radioactive ¹⁷⁷Lu and stable lutetium (¹⁷⁵Lu) at 2-, 4-, 6- and 8-fold excess of ^{175/177}Lu compared to the amount of DOTA-antibody (Table 11). Unconsumed lutetium was then complexed with an excess of EDTA to prevent the formation of insoluble lutetium hydroxide and the nonspecific attachment of lutetium to the antibody. For analysis 1 μL of the radioactive solution was placed on an ITLC strip and was eluted with 0.1M citrate buffer at pH 5. Autoradiography of TLC plates was obtained after exposure on a phosphorous screen (Typhoon IP, Amersham) using ImageQuant TL according to the volume integration. The number of DOTA molecules attached per DOTA-C0083 was estimated from the labelling efficiency and the molar ratio of protein to ^{175/177}Lu ions added into the solution.

TABLE 11
Labeling protocol for determination
of number of DOTA grafted to C0083

| | | |
|---|---|---|
| Ratio | Ratio 2 | 74.5 μg of DOTA-C0083 (0.517 nmol) |
| of molar | | 6 μL of NH₄Ac 250 mM pH 5.4 |
| equivalents | | 3 μL of ^{177/175}Lu 2 eq (1.024 nmol/1.0 μCi) |
| for ^{175/177}Lu/ | | 25 μL (Total volume) |
| DOTA-C0083 | Ratio 4 | 74.5 μg of DOTA-C0083 (0.517 nmol) |
| | | 6 μL of NH₄Ac 250 mM pH 5.4 |
| | | 3 μL of ^{177/175}Lu 4 eq |
| | | 25 μL (Total volume) |
| | Ratio 6 | 74.5 μg of DOTA-C0083 (0.517 nmol) |
| | | 6 μL of NH₄Ac 250 mM pH 5.4 |
| | | 3 μL of ^{177/175}Lu 6 eq |
| | | 25 μL (Total volume) |
| | Ratio 8 | 74.5 μg of DOTA-C0083 (0.517 nmol) |
| | | 6 μL of NH₄Ac 250 mM pH 5.4 |
| | | 3 μL of ^{177/175}Lu 8 eq |
| | | 25 μL (Total volume) |

In order to radiolabel DOTA-C0083 with ¹⁷⁷Lu at a specific activity of 275 MBq/mg, 81.4 MBq of ¹⁷⁷Lu chloride in 0.04 N HCl (ITG Germany) and 20 μL of ammonium acetate 0.25 M, pH 5.4 were added to 300 μg of DOTA-C0083. The reaction mixture was incubated for 10 minutes at 37° C. after which the radiolabeled conjugate incubated with 1 mM DTPA (final conc.) for additional 5 minutes to complex excess of 177Lu. The chelation efficiency was evaluated by ITLC eluting in citrate buffer 0.1 M pH 5 after DTPA addition and autoradiography of TLC plates (Typhoon IP, Amersham) using ImageQuant TL according to the volume integration.

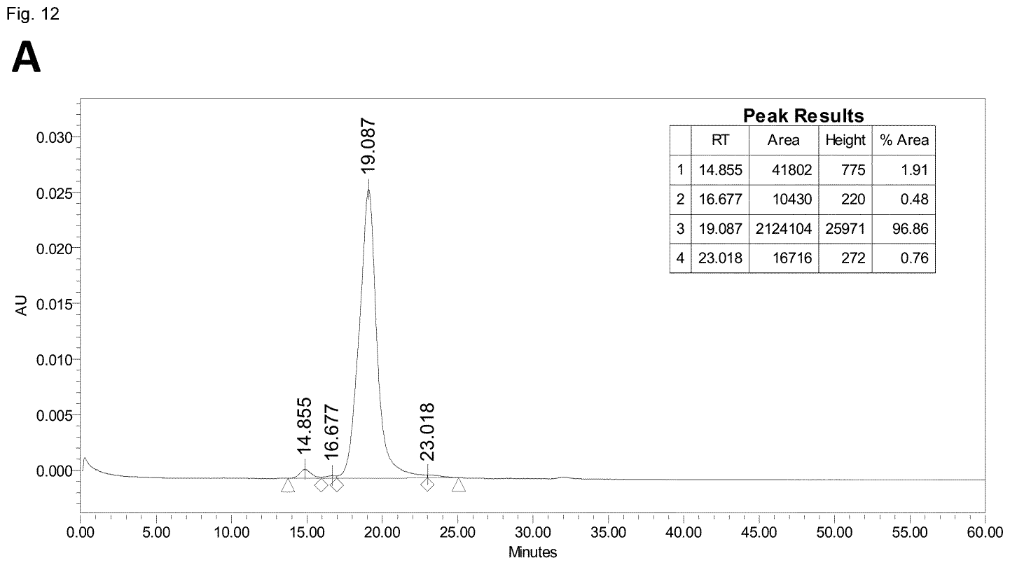

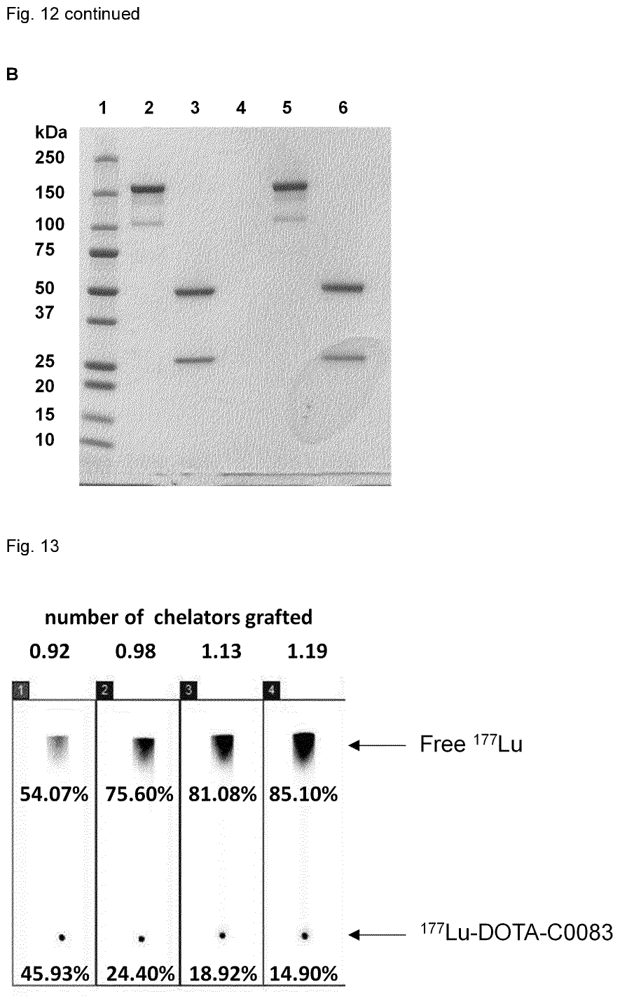

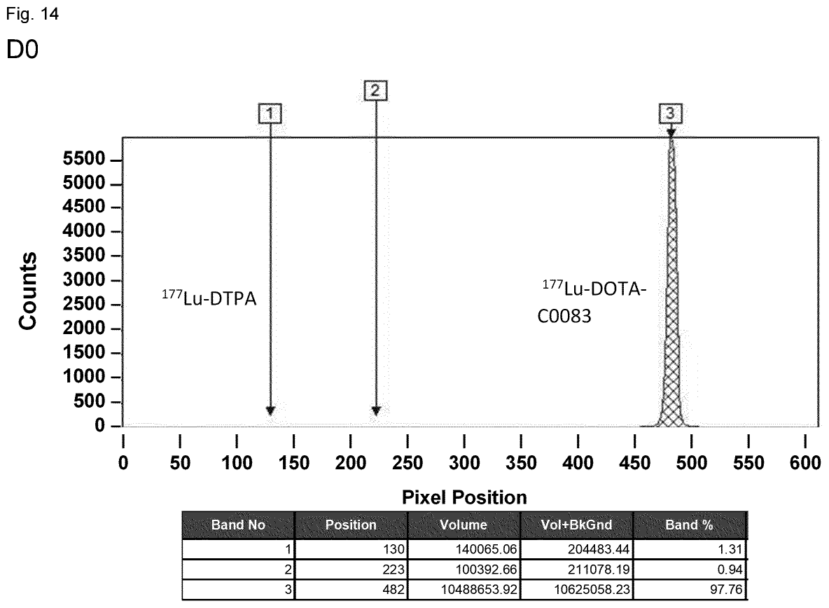

Results: Incubation of C0083 with SCN-Bn-DOTA and subsequent purification yielded a DOTA-C0083 conjugate of high chemical purity of ˜97%, as shown by SE-HPLC (FIG. 12A) and SDS PAGE (FIG. 12B). The resultant DOTA-C0083 conjugate was analyzed for the number of DOTA molecules attached per antibody and results from the ITLC are shown in FIG. 13. An average of about 1 DOTA molecule per antibody was obtained under the chosen conditions. DOTA-C0083 conjugate was then labelled successfully with lutetium-177 and the resultant radioimmunoconjugate showed a specific activity of 7.5 mCi/mg (277 MBq/mg) with an overall radiochemical purity of 97.8%, as determined by ITLC autoradiography (FIG. 14).

Conclusion: An efficient method to radiolabel the newly designed anti-oxMIF antibody C0083 with ¹⁷⁷Lu was established. The resultant radioimmunoconjugate showed excellent purity and specific activity, comparable to C0008. Additionally, the DOTA-C0083 conjugate, like its naked antibody (C0083), showed reduced hydrophobicity compared to C0008 determined by SEC analysis (FIG. 12A, retention time 19.1 min vs. FIG. 1A retention time 20.8 min).

FIG. 12 shows the purity and integrity analysis of DOTA-C0083; A: DOTA-C0083 SE-HPLC profile (λ=280 nm). B: Coomassie stained SDS PAGE: 1: molecular weight standard; 2: C0083 non-reduced; 3: C0083 reduced; 5: DOTA-C0083 non-reduced; 6: DOTA-C0083 reduced.

FIG. 13 shows the determination of the number of DOTA molecules grafted to C0083 determined by ITLC: ITLC separation of C0083-DOTA which was incubated with a mixture of tracer amounts of radioactive ¹⁷⁷Lu and stable lutetium (¹⁷⁵Lu) in 0.04 N HCl at 2 (lane 1), 4 (lane 2), 6 (lane 3) or 8 (lane 4) fold excess of ^{177/175}Lu in proportion to the amount of DOTA-antibody.

FIG. 14 shows the analysis of radiolabeled ¹⁷⁷Lu-DOTA-C0083 by ITLC and autoradiography: Autoradiogram of ITLC film (¹⁷⁷Lu-DOTA-C0083 after DTPA addition; Peak 1/2 reflects free ¹⁷⁷Lu-DTPA and 177Lu-DOTA-C0083 derivatives, Peak 3 reflects ¹⁷⁷Lu-DOTA-C0083).

### Example 11

Stability of ¹⁷⁷Lu-DOTA-C0083 in formulation buffer

### Materials and Methods

A solution of ¹⁷⁷Lu-DOTA-C0083 was prepared to achieve a final concentration of 200 μg/mL suitable for stability studies. The characteristics of the ¹⁷⁷Lu-DOTA-C0083 solution is presented in Table 12.

TABLE 12
Characteristics of ¹⁷⁷Lu-DOTA-C0083 solution

| Dosing solution | ¹⁷⁷Lu-DOTA-C0083 |
|---|---|
| Specific Activity | 7.286 mCi/mg (269.58 MBq/mg) |
| Radioactive concentration | 1.409 mCi/mL (42.13 MBq/mL) |
| Concentration mAb | 0.189 mg/mL in formulation buffer |
| Final Radiopurity ITLC | 97.76% |
| Buffer | 0.03M phosphate buffer pH 7 |
| | 0.08 mg/mL sodium Ascorbate |
| | 0.2 mg/mL MSA |
| | 0.07 mM DTPA |

¹⁷⁷Lu-DOTA-C0083 in formulation buffer stored at 4° C. was analyzed by ITLC and SE-HPLC at different time points. Radiochemical purity was determined by ITLC eluent with citrate 0.1 M pH 5, in order to quantify the percentage of radioactivity release from the radioimmunoconjugate over time. SE-HPLC was conducted on a Superdex 200, 10/300 GL column eluted with phosphate buffer containing arginine at a flow rate of 0.6 mL/min to determine aggregation and contaminants (i.e. ¹⁷⁷ Lu-DTPA or ¹⁷⁷Lu-DOTA). The chromatographic profiles were acquired with a Radiomatic 150TR flow scintillation analyser.

In addition, the radioactivity of the radioimmunoconjugate was measured by gamma counting to give information about the solubility of ¹⁷⁷Lu-DOTA-C0083 in formulation buffer. For this purpose, 5 μL were counted directly from 1/20 dilution in formulation buffer.

Results: ITLC analysis of the ¹⁷⁷Lu-DOTA-C0083 revealed almost no release of radioactivity from the radioimmunoconjugate over time. After 7 days, the solution of radiolabeled antibody presented a radiopurity of 94%. The radiolabeled antibody released about 4% of the initial bound radioactivity (Table 13) when kept at 4° C. in formulation buffer (Table 13). The ¹⁷⁷Lu-DOTA-C0083 eluted at 19.1 min and its contaminants (¹⁷⁷Lu-DTPA and ¹⁷⁷Lu-DOTA derivatives) eluted at 28.6 min. There was a slight increase for LMW species from day 0 to day 7, but no increase in aggregates was detected (Table 15). After 7 days, the dosing solution presented 93% intact antibody by SE-HPLC. Thus, the SE-HPLC results were in accordance with ITLC results. The radioactivity monitored over time was in good agreement with the theoretical radioactive decay for lutetium-177 (values in brackets in Table 14).

Conclusion: The ¹⁷⁷Lu-DOTA-conjugate of the newly designed anti-oxMIF antibody C0083 was stable in formulation buffer up to 7 days when tested with ITLC and SE-HPLC.

TABLE 13
ITLC - Radiopurity of ¹⁷⁷Lu-DOTA-C0083
in formulation buffer at 4° C.

| Time point | ¹⁷⁷Lu-DOTA-C0083 %* |
|---|---|
| D 0 | 97.76% |
| D 1 | 97.34% |
| D 3 | 96.15% |
| D 7 | 93.69% |

*percentage of antibody-associated ¹⁷⁷Lu, corresponding to intact protein, protein fragments and aggregates.

TABLE 14
Radioactive concentration of ¹⁷⁷Lu-DOTA-C0083
in formulation buffer at 4° C.

| Time point | Radioactivity |
|---|---|
| D 0 | 1.41 mCi/mL |
| D 3 | 1.03 mCi/mL (1.01 mCi/mL) |
| D 7 | 0.68 mCi/mL (0.67 mCi/mL) |

TABLE 15
Stability of ¹⁷⁷Lu-DOTA-C0083 in formulation buffer (SE-HPLC)

| Time point | SE-HPLC results |
|---|---|
| D 0 | 95.62% (intact Ab) |
| | 1.62% HMW |
| | 2.76% LMW |
| D 3 | 94.74% (intact Ab) |
| | 1.90% HMW |
| | 3.36% LMW |
| D 7 | 92.52% (intact Ab) |
| | 2.01% HMW |
| | 5.48% LMW |

HMW: high molecular weight impurities as aggregates and oligomers,
LMW: low molecular weight impurities as ¹⁷⁷Lu-DTPA or ¹⁷⁷Lu-DOTA derivatives

### Example 12

Stability of ¹⁷⁷Lu-C0083 in mouse plasma

Materials and Methods: Plasma stability of 177Lu-DOTA-C0083 was assessed using fresh mouse plasma (prepared with heparin lithium tubes) at 37° C. The concentration of ¹⁷⁷Lu-DOTA-C0083 in plasma was 20 μg/mL. The stability in plasma was evaluated at day 0, day 1, day 3 and day 7 by ITLC (eluting in citrate 0.1 M pH 5), autoradiography and SDS-PAGE.

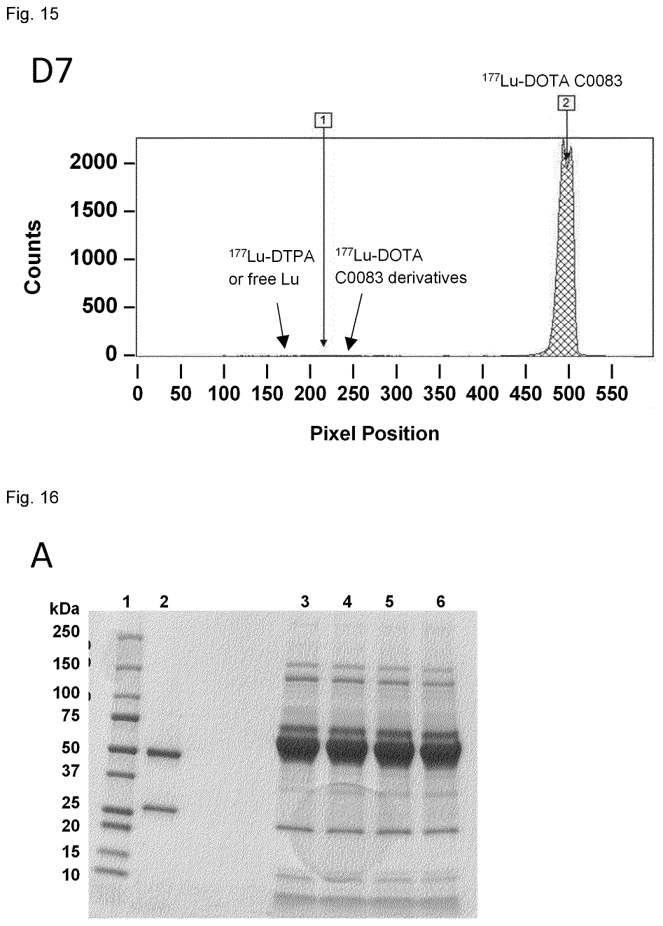

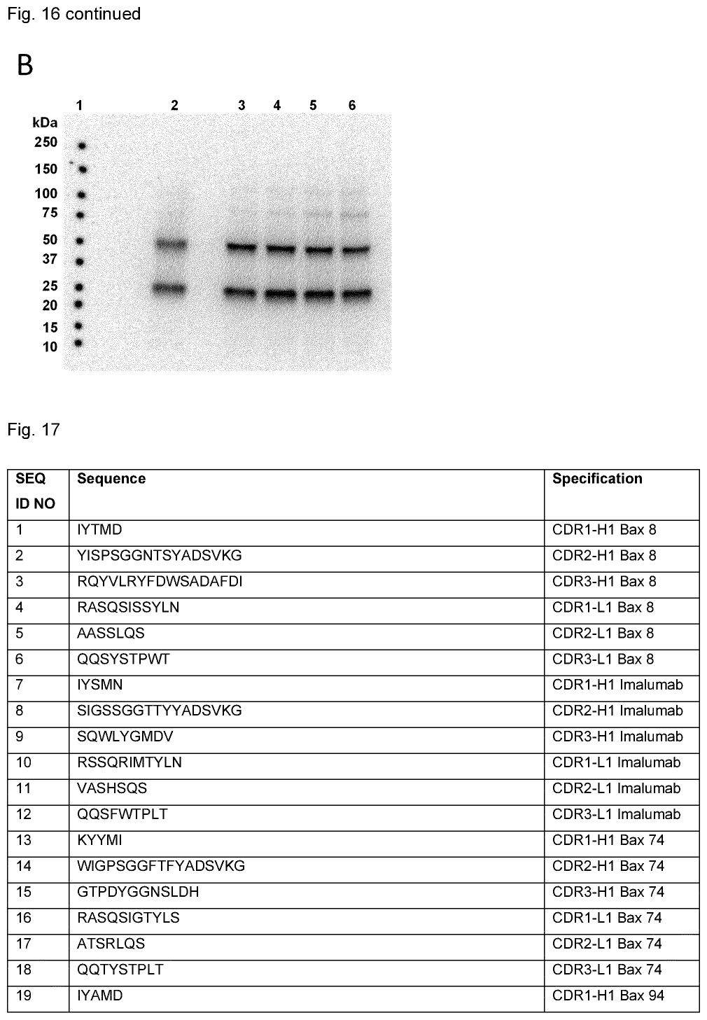

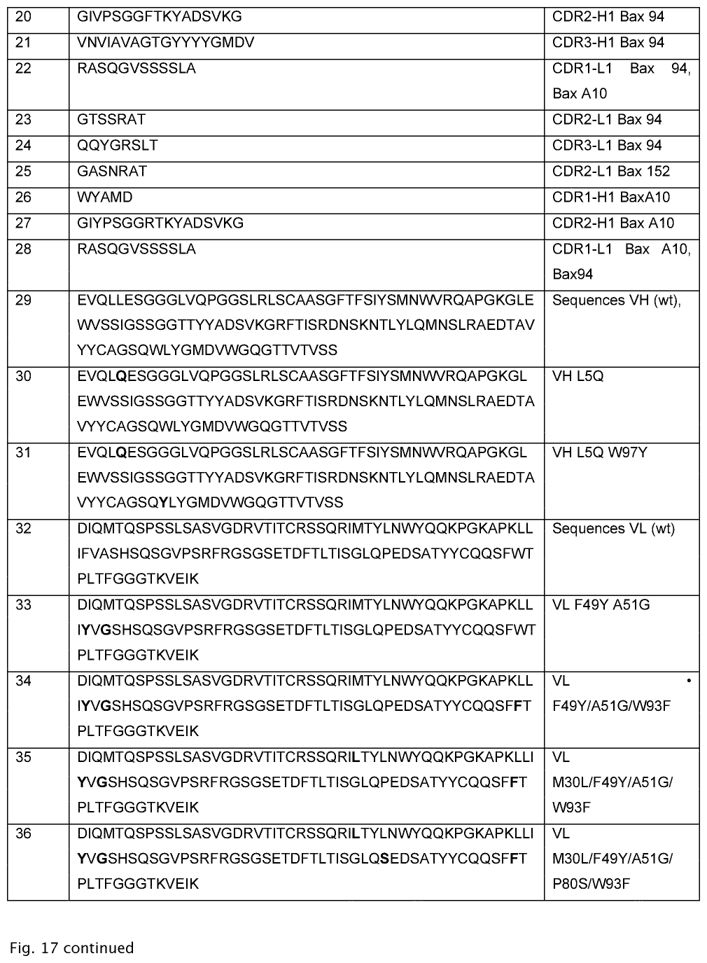

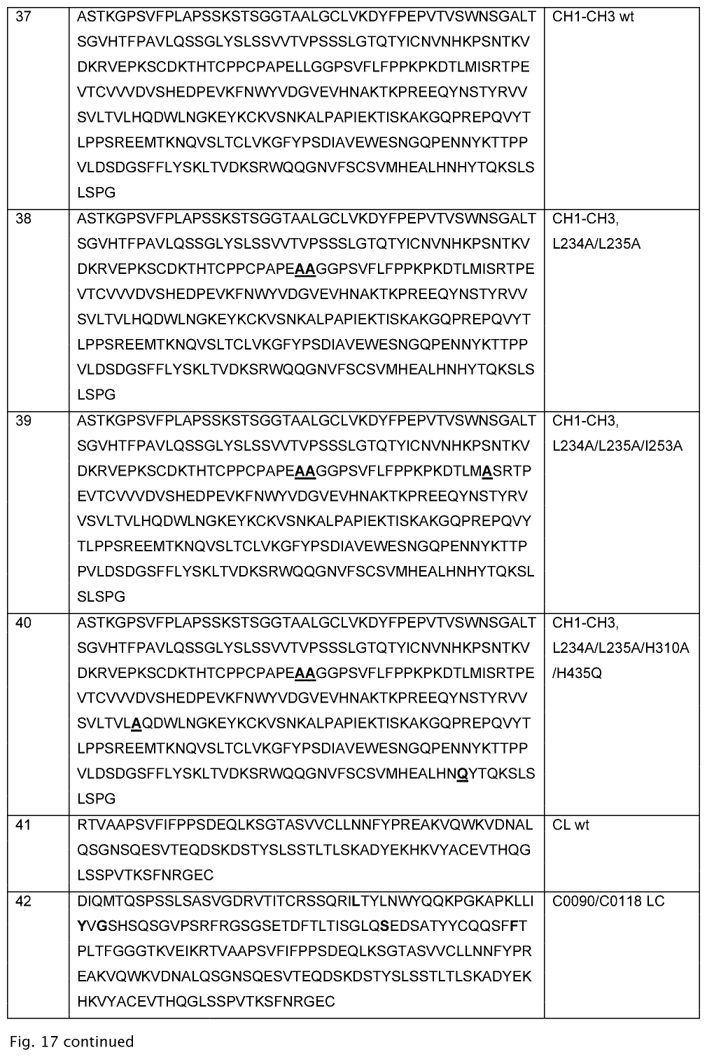

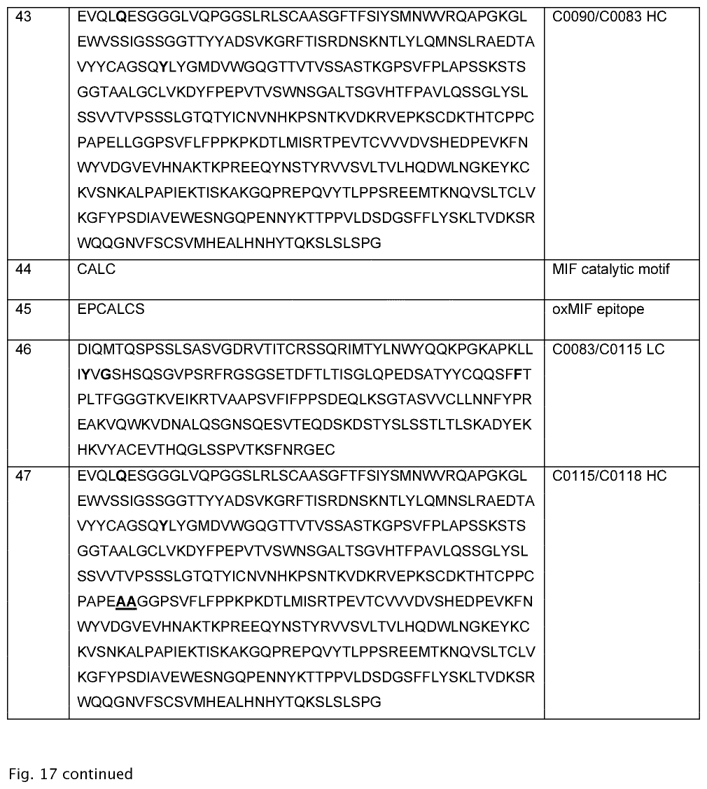

Results: After 7 days of incubation in mouse plasma at 37° C., the solution of radiolabeled antibody presented a radiopurity of about 96% with a corresponding release of only 1% of the initial bound radioactivity (Table 16). The released radioactivity corresponded to ¹⁷⁷Lu-DOTA-C0083 derivatives and free ¹⁷⁷Lu as demonstrated by ITLC results (FIG. 15). SDS-PAGE analyses revealed no significant degradation of the radiolabeled antibody when incubated in plasma (FIG. 16). In addition, no trans-chelation of ¹⁷⁷Lu to other plasma proteins was observed (FIG. 16).

Conclusion: The stability of ¹⁷⁷Lu-DOTA C0083 in plasma was excellent with only 1% ¹⁷⁷Lu activity released and no degradation products present after 7 days at 37° C. The reduction in hydrophobicity of the newly designed molecule C0083 also resulted in greatly enhanced plasma stability of the resulting ¹⁷⁷Lu-DOTA-C0083 radio-immunoconjugate compared to ¹⁷⁷Lu-DOTA-C0008 (1% vs. 20% release of initial bound radioactivity after 7 days for ¹⁷⁷Lu-DOTA-C0083 and ¹⁷⁷Lu-DOTA-C0008 (Example 3), respectively).

TABLE 16
Stability of ¹⁷⁷Lu-DOTA-C0083 in mouse plasma (ITLC)

| Time point | ¹⁷⁷Lu-DOTA-C0083 % |
|---|---|
| D 0 | 97.36% |
| D 1 | 97.01% |
| D 3 | 96.01% |
| D 7 | 96.26% |

FIG. 15 shows the plasma stability of ¹⁷⁷Lu-DOTA-C0083 (autoradiography): Autoradiogram of ¹⁷⁷Lu-DOTA-C0083 incubated in mouse plasma for 7 days; 1: ¹⁷⁷Lu-DOTA-C0083 derivatives and free ¹⁷⁷Lu; 2: ¹⁷⁷Lu-DOTA-C0083.

FIG. 16 shows the plasma stability of ¹⁷⁷Lu-DOTA-C0083 (SDS-PAGE): Reduced SDS PAGE with ¹⁷⁷Lu-DOTA-C0083 in mouse plasma (left: Coomassie staining; right: Autoradiography). Lane 1: molecular weight standard; Lane 2: ¹⁷⁷Lu-DOTA-C0083 in buffer (TO); Lane 3: ¹⁷⁷Lu-DOTA-C0083 (TO hours in plasma); Lane 4: ¹⁷⁷Lu-DOTA-C0083 (1 day in plasma), Lane 5: ¹⁷⁷Lu-DOTA-C0083 (3 days in plasma); Lane 6: ¹⁷⁷Lu-DOTA-C0083 (7 days in plasma).

### Example 13: Reduced Aggregation Propensity and Reduced Hydrophobicity of Newly Designed Anti-oxMIF Antibodies with Fc Silencing

To evaluate hydrophobicity and aggregation, the newly designed Fc-silenced antibodies C0115 and C0118 were analyzed by gel filtration (SEC) using SEC column, and by hydrophobic interaction chromatography (HIC) compared to the control anti-oxMIF antibody C0008 and their parent antibodies C0083 and C0090 without Fc silencing.

For SEC, samples were diluted to 1 mg/ml in 1x phosphate buffered saline (1xPBS) and 100 μl sample was applied to an Enrich 650 (Bio-Rad) gel-filtration column at a flow rate of 1.25 ml/min. Separations and equilibration was performed in 1×PBS at room temperature. Protein peaks were monitored using absorbance at 280 nm and spectra were analyzed using the ChromLab software package (Bio-Rad). Results are reported in retention volume (Vr, ml) for the main peak and presence of aggregates was ranked manually.

For hydrophobic interaction chromatography (HIC) analysis, all samples were diluted to a final concentration of 1 mg/ml using 50 mM phosphate and 0.75 M Ammonium Sulphate, pH 6.9. Highly purified samples of antibodies (˜100 μg) were loaded independently onto a 1 ml HiTrap Butyl HP column. 100 μl samples were injected and the column flow rate was maintained at 1 ml/min at 22° C. Separation of peaks was carried out over a 20-column volume (CV) gradient from 0-100% B (Buffer B: 50 mM phosphate, 20% isopropanol; pH 7.0). Protein peaks were monitored using absorbance at 280 nm and spectra were analyzed using the ChromLab software package (Bio-Rad).

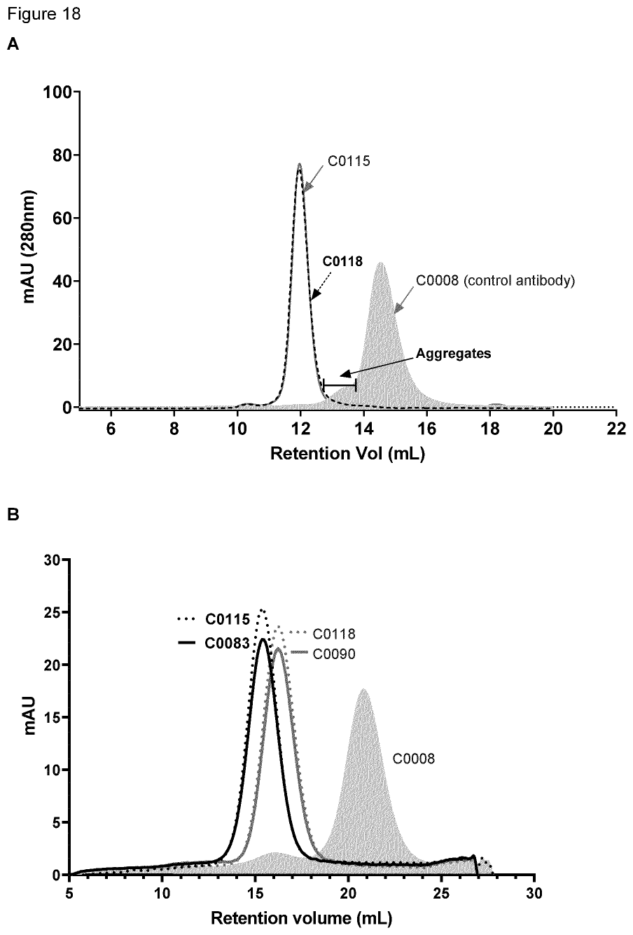

Results: Control antibody C0008 demonstrated a retention volume close to the void volume of the size exclusion column (˜15 ml Enrich 650), which corresponds to a molecular weight far smaller than expected for a human IgG (FIG. 18A). The unusual long retention was mainly due to hydrophobic interactions with the stationary phase surface. In addition, to the high retention volume, significant amounts of IgG dimers and aggregates were present for C0008. The newly designed antibodies C0115 and C0118 showed a retention volume corresponding to the molecular weight of a monomeric human IgG, when compared to a molecular weight standard (Table 17, FIG. 18A). Additionally, antibody dimers and aggregation were significantly reduced in samples of newly designed antibodies C0115 and C0118 (FIG. 18A). The Fc silencing mutations of the new newly designed antibodies C0115 and C0118, did not alter their SEC profile compared to their parent antibodies C0083 and C0090 with wt Fc, respectively (FIG. 18A, and FIG. 7B from Example 6).

HIC column retention volumes are a measure of hydrophobicity where antibodies with low retention volume are less hydrophobic than antibodies with high retention volume (FIG. 18 B). The newly designed Fc-silenced antibodies C0115 and C0118 demonstrated to be less hydrophobic (retention volume of 15-16 ml), compared the control antibody C0008 (retention volume of ˜21 ml) showing a very high hydrophobicity. The Fc silencing mutations of the new newly designed antibodies C0115 and C0118, did not alter their HIC profile compared to their parent antibodies C0083 and C0090 with wt Fc, respectively.

Conclusion: It is evident from SEC and HIC analysis that the newly designed Fc-silenced antibodies have improved biochemical properties, in particular reduced hydrophobicity, and aggregation propensity compared to the control anti-oxMIF antibody C0008.

TABLE 17
Size Exclusion Chromatography

| Sample | Running Buffer | Column | Vr (ml) | Aggregates |
|---|---|---|---|---|
| C0008 | 1xPBS | Enrich 650 | 18.06 | + |
| C0083 | 1xPBS | Enrich 650 | 12.39 | − |
| C0090 | 1xPBS | Enrich 650 | 12.38 | − |
| C0115 | 1xPBS | Enrich 650 | 11.96 | − |
| C0118 | 1xPBS | Enrich 650 | 11.95 | − |

FIG. 18 shows the chromatography profiles demonstrating reduced aggregation and hydrophobicity of the newly designed Fc silenced anti-oxMIF antibodies. (A) Comparison of elution profiles of C0008 (control antibody, gray area) and newly designed Fc silenced antibodies C0115 and C0118 on a Enrich 650 gel filtration column using 1xPBS as mobile phase (B) Comparison of elution profiles of C0008 (control antibody, gray area) and newly designed Fc silenced antibodies C0115 and C0118 and their parent antibodies C0083 and C0090 (without Fc silencing) on a HiTrap Butyl HP HIC column.

### Example 14: Binding of Newly Designed Fc Silenced Anti-oxMIF Antibodies C0115 and C0118 with and without Conjugation to DFO* to Immobilized MIF (KD Determination)

Recombinant human MIF diluted in PBS at 1 μg/ml was immobilized into ELISA plates overnight at 4° C. (transforming MIF to oxMIF according to Thiele et al., 2015). After blocking, serial dilutions of anti-oxMIF antibodies with and without DFO* conjugation (DFO* conjugation is described in Example 19) were added to the plates. Finally, bound antibodies were detected using a Goat anti-human IgG (Fc)-HRP conjugate and tetramethylbenzidine (TMB) as substrate. The chromogenic reaction was stopped with 3M H₂SO₄ and OD was measured at 450 nm. If data from different experiments were combined, the data were normalized to the maximal OD of the anti-oxMIF antibody C0008 (=100%) of the respective experiment and EC₅₀ values were determined by 4-parameter fit using GraphPad Prism (and the mean+/−SEM of two experiments is shown).

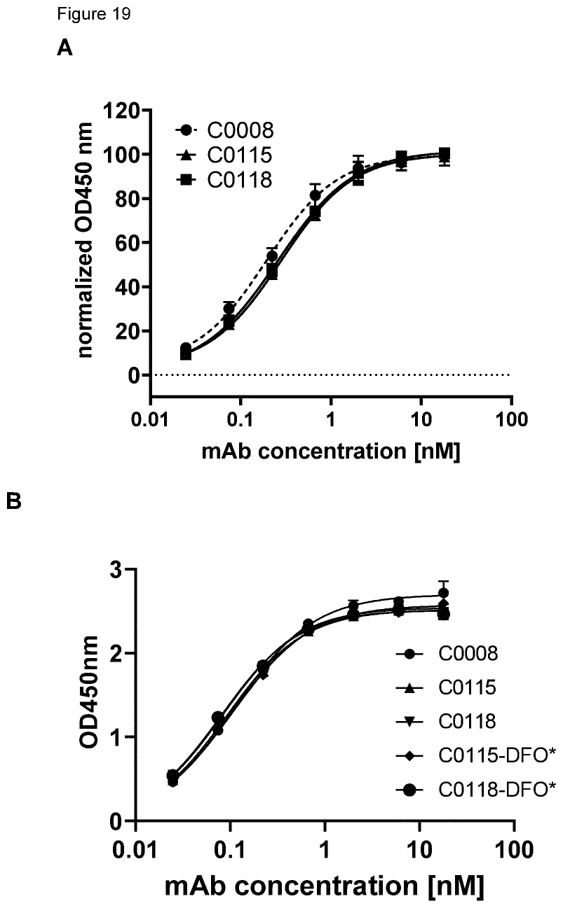

Results and conclusion: The binding of the newly designed antibodies towards immobilized MIF (oxMIF) was measured over a broad range of concentrations and the resulting binding curves are illustrated in FIG. 19. The anti-oxMIF antibody C0008 was used as reference for oxMIF binding. The binding curves and the calculated EC₅₀ values, representing the apparent KD, clearly showed that the newly designed Fc-silenced antibodies C0115 and C0118 retained their low nanomolar affinity to oxMIF, compared to C0008 (FIG. 19A, Table 18). Additionally, DFO* conjugation of the newly designed Fc-silenced antibodies C0115 and C0118 has not changed their affinity (FIG. 19B). This suggests that data obtained with the unconjugated antibodies as well as i.e. IRDye 800CW labeled antibodies can be directly translated into radioimmunoconjugates such as DFO *-Zr89 labeled antibodies.

FIG. 19 shows the binding curves of newly designed Fc silenced anti-oxMIF antibodies to immobilized oxMIF (K_{D} determination). Anti-oxMIF antibodies were detected by an anti-human-IgG (Fc)-HRP conjugate, C0008 was used as reference antibody.

TABLE 18
K_{D} values of newly designed Fc-silenced anti-
oxMIF antibodies C0115 and C0118 versus C0008.

| Sample | K_{D} (nM) |
|---|---|
| C0008 | 0.19 |
| C0115 | 0.28 |
| C0118 | 0.26 |

### Example 15: Differential Binding of Newly Designed Fc Silenced Anti-oxMIF Antibodies C0115 and C0118 to oxMIF Vs. redMIF

Material and methods: Binding of C0115 and C0118 to oxMIF versus redMIF was analyzed as described in the Example 8.

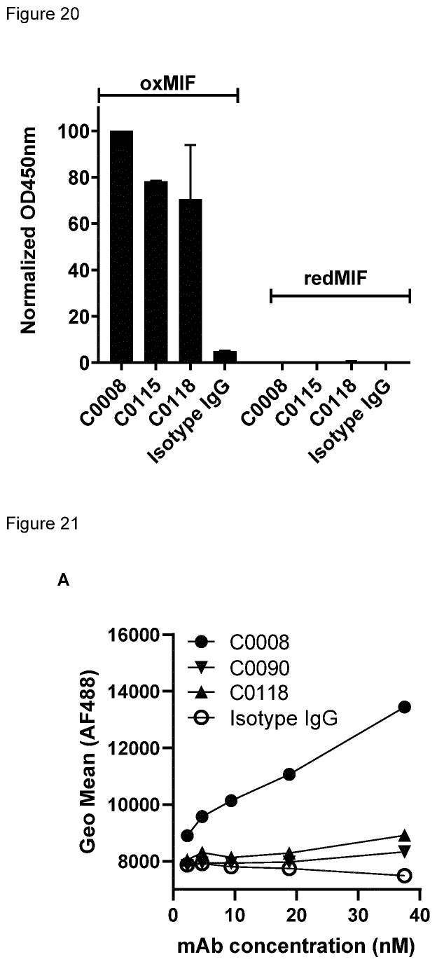

Results and conclusion: The binding of the newly designed Fc silenced antibodies C0115 and C0118 towards soluble oxMIF and redMIF are illustrated in FIG. 20. The results clearly showed that the newly designed Fc silenced antibodies C0115 and C0118 demonstrated strong binding to oxMIF but no binding to redMIF. C0115 and C0118 showed very comparable ODs to the reference antibody C0008, which has been described to discriminate between oxMIF and redMIF (Thiele et al., 2015). No binding was observed for the isotype IgG. Thus, the newly designed antibodies with reduced hydrophobicity and aggregation and Fc silencing retained their ability to discriminate between oxMIF and redMIF.

FIG. 20 shows the differential binding of the newly designed Fc silenced antibodies to oxMIF vs. redMIF. C0008 was used as reference antibody and an Isotype IgG as negative control.

### Example 16: Reduced Unspecific Binding of the Newly Designed Fc Silenced Anti-oxMIF Antibodies C0115 and C0118 to A2780 MIF^{-/-} Cells

A2780 MIF^{-/-} cell line was generated by CRISPR/Cas9 gene editing of human MIF gene in A2780 ovarian carcinoma cell line. In brief, target gene sequence was analysed and target sites were located according to the general rules of designing a targeting guidance RNA (gRNA) for GenCRISPR™ system. A guide RNA (gRNA) was designed to specifically recognize the 5′ region of the MIF gene (TTGGTGTTTACGATGAACATCGG, SEQ ID NO: 48) and the gRNA sequence was cloned into the PX459 (addgene) vector containing S. pyogenes Cas9 (SpCas9) nuclease. A2780 cells were transiently transfected by electroporation and were plated in 96-well plates by limit dilution to generate isogenic single clones. Isogenic single clones, where the endogenous MIF gene was efficiently mutated, resulting in consequential reduction (or removal) of the expression of the MIF protein were identified by Sanger sequencing screening. The final clone showed a deletion of 10 bp at position +2 after the start codon of the human MIF gene. Absence of endogenous human MIF protein in the A2780 MIF^{-/-} cell line was confirmed by Western blotting using polyclonal anti-human MIF antibodies.

A2780 MIF^{-/-} cells were detached with Cell Stripper (Corning, Cat #25-056-CI), washed with staining buffer (PBS+5% BSA) and plated into 96-well U-bottom plate at 2x105 cells per well. Cells were stained with fixable viability dye eFluor780 (Invitrogen, diluted 1:2000 in PBS) for 20 min at 4° C. and washed with staining buffer. Cells were resuspended in 50 μl of staining buffer, and 50 μl of serial dilutions of the newly designed anti-oxMIF Fc silenced antibodies C0115 and C0118 or their parent antibodies C0083 and C0090, respectively, or the control anti-oxMIF antibody C0008 or an isotype IgG (final concentrations 37 nM-9.4 nM) were added. After incubating for 40 min at 4° C., cells were washed with staining buffer, and resuspended in 100 μl of secondary antibody (goat anti-human IgG (H+L)-AlexaFluor 488, diluted 1:100). After incubating for 30 min at 4° C., cells were washed with staining buffer, resuspended in PBS+2% BSA and acquired on the Cytoflex-S flow cytometer (Beckman Coulter).

Data were analyzed with FlowJo and the GeoMean (mean fluorescence intensity for AF488), of viable cells was plotted against antibody concentration in GraphPad Prism.

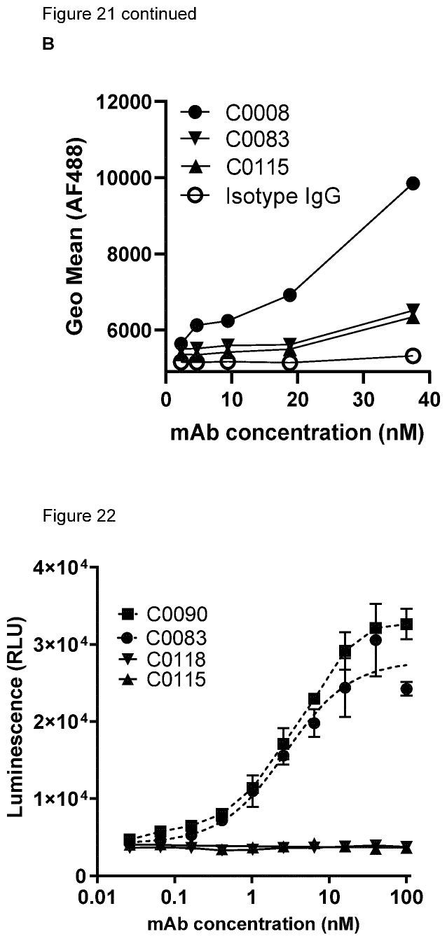

Results and Conclusion: It is apparent from FIGS. 21A and B that the newly designed Fc silenced anti-oxMIF antibody C0118 and its parent antibody C0090 (A) as well as C0118 and its parent antibody C0083 (B) do not bind to A2780 MIF^{-/-} cells, as their Geo Mean is very close to those of the isotype IgG negative control. In contrast, the anti-oxMIF antibody C0008 showed significant binding to A2780 MIF^{-/-} cells, although they were proven not to express MIF. Thus, reduction of hydrophobicity resulted in a strong reduction or elimination of unspecific binding of the newly designed anti-oxMIF antibodies to A2780 MIF^{-/-}, whereas the anti-oxMIF control antibody C0008 binds unspecifically to the cell surface due to its hydrophobic properties.

FIG. 21 shows the reduced unspecific binding of the newly designed anti-oxMIF antibodies on A2780 MIF^{-/-} cells determined by FACS. Staining of A2780 MIF^{-/-} cells with the newly designed Fc silenced anti-oxMIF antibody C0118 and its parent antibody C0090 (A) and C0115 and its parent antibody C0083 (B) and the control antibody C0008 as well as an isotype IgG as negative control; GeoMean (mean fluorescence intensity for AF488) of viable cells is plotted against antibody concentration.

### Example 17: Strongly Reduced ADCC Effector Functions of the Newly Designed Fc Silenced Anti-oxMIF Antibodies C0115 and C0118 Determined by Reporter Assays

As described above, efforts can be made to decrease ADCC potential of antibodies by point mutations in the Fc portion, i.e. L234A/L235A. When the Fc portion of target-bound antibodies binds to Fc receptors on the cell surface of effector cells, multiple cross-linking of the two cell types occurs, leading to pathway activation of ADCC, which is not desired for target neutralizing antibodies to prevent functional Fc related adverse events.

Using engineered Jurkat cells stably expressing the human FcγRIIIA receptor, V158 (high affinity allotype) and a NFAT response element driving expression of firefly luciferase as effector cells, antibody biological ADCC activity is quantified through the luciferase produced as a result of NFAT pathway activation.

The ADCC reporter assay was essentially performed as recommended by the manufacturer (Promega #G7010).

To generate highly responsive target cells, HCT116 cells were transfected with a huMIF-pDisplay plasmid (Invitrogen), selected with geneticin, and sorted by FACS to generate cell lines stably expressing membrane-anchored monomeric human MIF (HCT116-pMIF), i.e. MIF is displayed as monomeric protein in which an oxMIF epitope is accessible to anti-oxMIF antibodies (Schinagl et al., 2018). This cell line shows increased presentation of oxMIF at the cellular surface and is therefore a more sensitive tool for in vitro analysis.

In brief, 1×10⁴ cells/well of HCT116-pMIF target cells in 100 μl assay medium (RPMI 1640 medium supplemented with Pen/Strept/L-Glutamin and 4% low IgG FBS) were seeded into 96-well flat-bottom white plates and left to adhere overnight at 37° C./5% CO₂ in a humidified incubator. On the next day, medium was replaced by 25 μl of the fresh assay medium. Twenty-five μl of serial dilutions of the newly designed anti-oxMIF antibodies C0115 & C0118 with silenced Fc or their parent antibodies C0083, C0090 (final concentration 0.01-100 nM) and 25 μl of the freshly-thawed effector cells (FcγRIIIA receptor effector cells of high responder genotype V158) at effector to target cell ratio of ˜6:1 were added to the plates. After incubation for 6 h at 37° C./5° CO₂ in a humidified incubator, assay plates were equilibrated to room temperature and Bio-Glo Luciferase reagent was added. Luminescence (RLU) was measured after 10-20 min incubation using a Tecan multiplate reader (0.5 s integration time).

Results: It is evident from FIG. 22 that the newly designed antibodies C0115 & C0118 harboring Fc silencing mutations did not show any activation of the effector cells in the ADCC reporter assay, whereas their parent antibodies with wt Fc, C0083 and C0090 induced strong FcγRIIIA mediated activation of effector cells.

Conclusion: The newly designed variants C0115 & C0118 harboring Fc silencing mutations (L234A/L235A) did not show any initiation of ADCC in a reporter bioassay. Thus, they show strongly reduced ADCC effector functions.

FIG. 22 shows the strongly reduced ADCC effector function of the newly designed Fc silenced antibodies C0115 & C0118 determined by reporter assays. ADCC reporter bioassay with the newly designed Fc silenced antibodies C0115 and/or C0118 using engineered Jurkat effector cells stably expressing FcγRIIIA and HCT116-pMIF target cells compared to their parent antibodies C0083 and C0090 with wt Fc. Mean and SEM are shown (n=2).

### Example 18: Biodistribution of Newly Designed Fc-Silenced Anti-oxMIF Antibody C0115 and Control Antibody C0008 in HCT116 Tumor Bearing Balb/c Mice Nude Mice

Materials and Methods: Biodistribution of newly designed Fc-silenced anti-oxMIF antibody C0115 was investigated in a head-to-head comparison to the reference anti-oxMIF antibody C0008 in a human colon adenocarcinoma cell line HCT116 xenograft mouse model. Female Balb/c nude mice received unilateral, subcutaneous injections of 5×10⁶ HCT116 cells in 50% PBS and 50% matrigel in a total injection volume of 100 μl. Upon reaching individual tumor volumes of 150-300 mm³, mice were assigned to treatment groups and received a single intravenous dose of 5 mg/kg IRDye 800CW-labeled C0115 or C0008.

C0115 and C0008 were labelled with IRDye 800CW using the IRDye 800CW Protein labeling kit-high MW from LI-COR Biosciences following the manufacturer's instructions. After the labelling process and prior to injection of labeled antibodies into mice, the protein concentration and labeling efficiency of the IRDye 800CW labeled antibody was determined using the Nanodrop technology, and mice were dosed based on the protein concentration after labelling. In vivo imaging was performed in a LI-COR Pearl® Trilogy imaging device upon administration of labelled antibodies at the following time-points: 1 h, 6-8 h, 24 h, 48 h, 72 h, 96 h, 168 h after dosing. Image analysis was performed to quantify the relative fluorescence intensity (RFU) of the antibody in the tumor (RFU/area=RFU tumor area/mm² tumor area−RFU background/mm² background area)

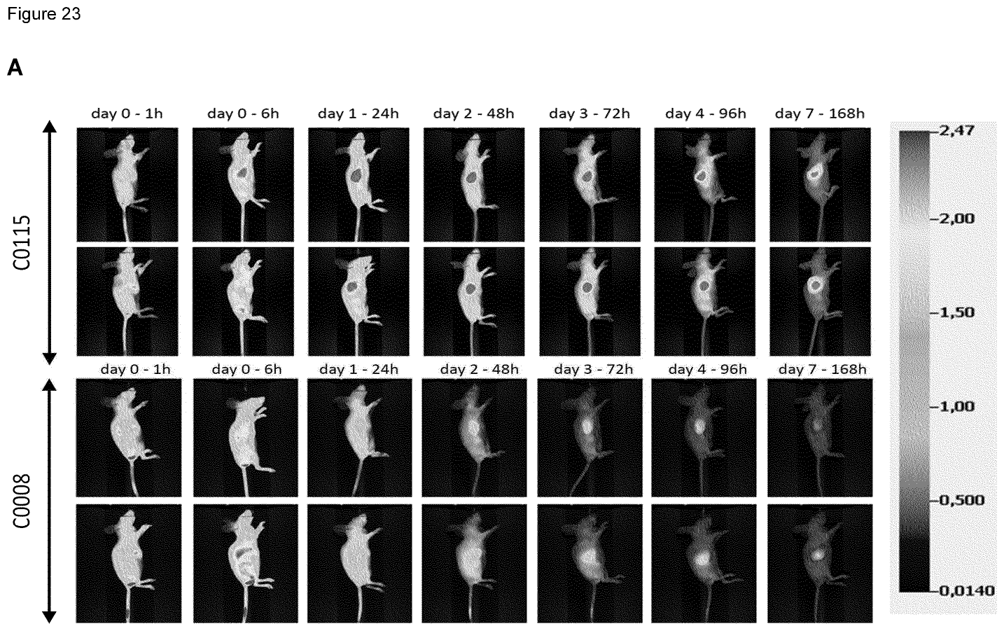

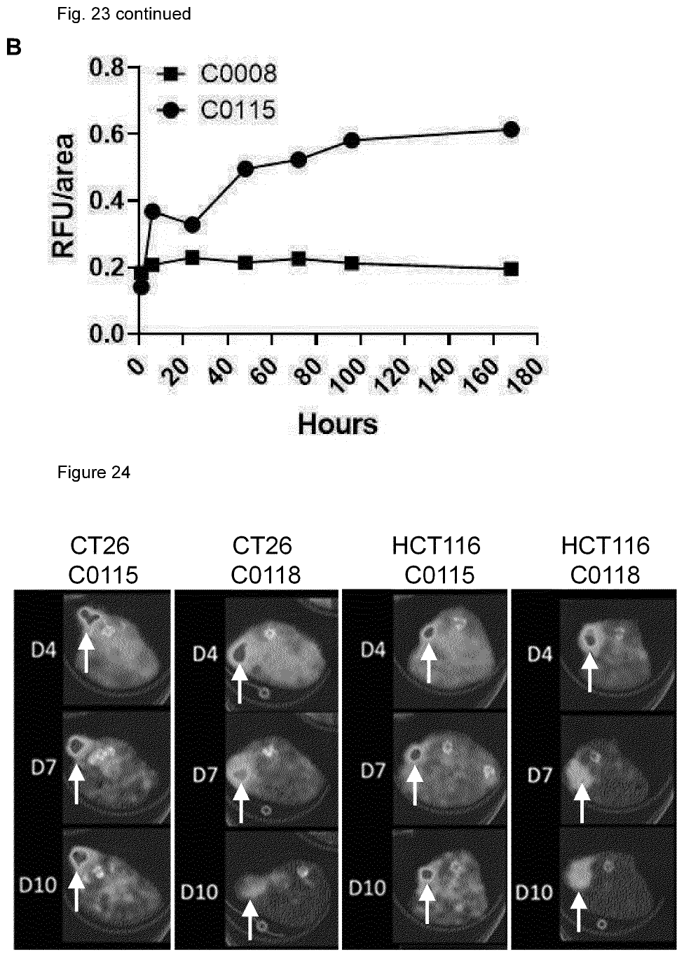

Results and Conclusion: FIG. 23 shows a significant intra-tumoral distribution of intravenously administered IRDye 800CW-labeled C0115 and C0008, respectively, with tumor retention up to 7 days. It is evident from FIG. 23A and quantitative image analysis (FIG. 23B) that the tumor uptake of newly designed Fc silenced anti-oxMIF antibody C0115 was strongly enhanced and increased over 7 days compared to the reference anti-oxMIF antibody C0008, which showed a peak at ˜24 h. Thus, the biodistribution study suggests the superiority of C0115 compared to C0008 for application as radioimmunoconjugate.

FIG. 23 shows the tumor penetration and retention of the newly designed anti-oxMIF Fc silenced antibody C0115 and the reference antibody C0008 by infra-red in vivo imaging of mice carrying subcutaneous HCT116 tumors. (A) Infra-red images of mice were taken 1 h, 6 h, 24 h, 48 h, 72 h, 96 h and 168 h post injection of the IRDye 800CW labeled antibodies C0115 (upper panel) and C0008 (lower panel) dosed at 5 mg/kg; (B) tumor penetration and retention of C0115 and C0008 quantified by digital image analysis.

### Example 19: Biodistribution of Newly Designed Fc-Silenced Anti-oxMIF Antibodies C0115 and C0118 Conjugated to DFO* and Radiolabeled with 89Zr in CT26 or HCT116 Tumor Bearing Balb/c or Balb/c Nude Mice

Materials and Methods: Biodistribution of newly designed Fc-silenced anti-oxMIF antibodies ⁸⁹Zr-DFO*-C0115 and ⁸⁹Zr-DFO*-C0118 were investigated by using a human colon adenocarcinoma cell line HCT116 xenograft and mouse colon adenocarcinoma cell line CT26 syngraft mouse model.

Antibodies C0115 and C0118 were conjugated with the bi-functional chelator DFO*-NCS (ABX advanced biochemical compounds GmbH). The buffer of antibodies was exchanged to 0.9% NaCl, 50 mM NaHCO₃, pH 9 (5.0 mg/mL). Thereafter the antibodies were reacted with 3 molar equivalents (excess) of DFO*-NCS in DMSO (1 mg/ml) at 37° C. for 45 min. After the reaction antibodies were purified with HiTrap-column (desalting column, GE 17-1408-01, 5 ml). During the purification the buffer was exchanged to DPBS w/o Ca²⁺ and Mg²⁺.

Radiolabeling of DFO*-Abs was performed with 89Zr (Perkin Elmer). First, approximately 100 MBq 89Zr (in 1 M oxalic acid) was mixed with 180 μl of 2M Na₂CO₃ solution and let to react for 3 min in room temperature (RT). The 89Zr solution was neutralized to pH 7 with 1 ml HEPES (pH 7) and 2 mg of DFO*-antibodies were added to the mixture and let to react 60 min at RT. The radiolabelled antibodies were purified with centrifugal filters and buffer exchanged to 0.01M PBS pH 6.5 with 50 mM Arginine. The radiochemical purity (RCP) was analyzed directly after the radiolabeling with iTLC using 0.05M DTPA as eluent and radio-HPLC (Agilent Infinity II, 1260) with UV detector (280 nm), Posiram radiodetector (Lablogic), size exclusion column SEC-2000 (Phenomenex, set to 30° C.), and 0.1M sodium phosphate pH 6.8 as mobile phase.

Female Balb/c nude mice received unilateral, subcutaneous injections of 5×10⁶ HCT116 cells in 50% PBS and 50% matrigel in a total injection volume of 100 μl. Female Balb/c mice received unilateral, subcutaneous injections of 3×10⁵ CT26 cells in 100 μl of PBS. Upon reaching individual tumor volumes of 150-250 mm³, mice were assigned to treatment groups and received a single intravenous dose of 10-11 MBq ⁸⁹Zr-DFO*-C0115 and ⁸⁹Zr-DFO*-C0118 antibodies. The radioactivity dose was determined by measuring the syringe pre- and post-dosing with cross calibrated dose calibrator (VDC-405, Veenstra instruments).

The animals were anesthetized with isoflurane and whole-body PET scanning (60 min) was performed 4-, 7- or 10-days post injection with BioPET small animal PET/CT (Sedecal, Madrid Spain) followed by CT image for anatomical reference. Images were reconstructed with 2DOSEM algorithm with CT-based attenuation correction. Image analysis was performed with PMOD software (v 3.7, PMOD Technologies LLC, Zürich, Switzerland).

Results and Conclusion: Both antibodies were successfully DFO*-modified and retained their affinity to oxMIF (FIG. 19B). ⁸⁹Zr-DFO*-C0115 was synthesized with approximately 80% radiochemical yield and a calculated specific activity of 19.7 MBq/mg. Injected radioactivity was 11.0±0.5 MBq (mean±SD, n=6) and injected mass was 0.56±0.03 mg (mean±SD, n=6). ⁸⁹Zr-DFO*-C0118 was synthesized with approximately 72% radiochemical yield and a calculated specific activity of 54.0 MBq/mg. Injected radioactivity was 11.0±0.2 MBq (mean±SD, n=6) and injected mass was 0.20±0.01 mg (mean±SD, n=6). It is evident from FIG. 24 that both newly designed Fc silenced anti-oxMIF antibodies accumulated in the tumor of both animal models and were retained in the tumor for at least 10 days. Thus, the biodistribution study suggests the suitability of the newly designed Fc silenced antibodies C0115 and C0118 for application as a diagnostic radioimmunoconjugate.

FIG. 24 shows the tumor penetration and retention of the newly designed anti-oxMIF antibodies C0115 and C0118 conjugated to DFO* and radiolabeled by ⁸⁹Zr by in vivo PET/CT imaging of mice carrying subcutaneous CT26 or HCT116 tumors. PET and CT images of mice were taken 4-, 7- and 10-days post injection of the ⁸⁹Zr-DFO* labeled antibodies dosed at ˜10 MBq per mouse; Representative digital vertical cross sections of the mice are shown, and the white arrows mark the tumor.

### REFERENCES

Bloom B. R. and Bennet, B., 1966, Mechanism of a reaction in vitro associated with delayed-type hypersensitivity, Science 153, 80-82
Brechbiel M. W. (2008). Bifunctional chelates for metal nuclides. The quarterly journal of nuclear medicine and molecular imaging: official publication of the Italian Association of Nuclear Medicine (AIMN) [and] the International Association of Radiopharmacology (IAR), [and] Section of the Society of . . . , 52(2), 166-173.
Brinkmann U. and Kontermann R. E., 2017, The making of bispecific antibodies, MABS, 9, 2, 182-212
Brüggemann, M, et al., 1987, Comparison of the effector functions of human immunoglobulins using a matched set of chimeric antibodies, J. Exp. Med. 166 1351-1361
Chomet, M., et al., (2021). Head-to-head comparison of DFO* and DFO chelators: selection of the best candidate for clinical ⁸⁹Zr-immuno-PET. European Journal of Nuclear Medicine and Molecular Imaging, 48(3), 694-707. https://doi.org/10.1007/s00259-020-05002-7
Schinagl, A., Kerschbaumer, R. J., Sabarth, N., Douillard, P., Scholz, P., Voelkel, D., Hollerweger, J. C., Goettig, P., Brandstetter, H., Scheiflinger, F., & Thiele, M. (2018). Role of the Cysteine 81 Residue of Macrophage Migration Inhibitory Factor as a Molecular Redox Switch. Biochemistry, 57(9), 1523-1532. https://doi.org/10.1021/acs.biochem. 7b01156
Clynes, et al., 1998, Fc receptors are required in passive and active immunity to melanoma, Proc. Nat'l Acad. Sci. USA 95 652-656.
David, J. R., 1966, Delayed hypersensitivity in vitro: its mediation by cell-free substances formed by lymphoid cell-antigen interaction, Proc. Natl. Acad. Sci. U.S.A. 56, 72-77
Carter L. M. et al., 2018, Preclinical optimization of antibody-based radiopharmaceuticals for cancer imaging and radionuclide therapy-Model, vector, and radionuclide selection, J Labelled Comp Radiopharm. 61(9), 611-635
Cragg, M. S., et al., 2003, Complement-mediated lysis by anti-CD20 mAb correlates with segregation into lipid rafts, Blood 101 1045-1052
Cragg, M. S., and Glennie, M. J., 2004, Antibody specificity controls in vivo effector mechanisms of anti-CD20 reagents, Blood 103 (2004) 2738-2743
Estep P. et al., 2015, An alternative assay to hydrophobic interaction chromatography for high-throughput characterization of monoclonal antibodies, MAbs, 7(3), 553-561
Ewert S. et al., 2003, Structure-based improvement of the biophysical properties of immunoglobulin VH domains with a generalizable approach, Biochemistry, 18, 42(6), 1517-28
Hellstrom, L, et al., 1986, Antitumor effects of L6, an IgG2a antibody that reacts with most human carcinomas, Proc. Nat'l Acad. Sci. USA 83 7059-7063
Hellstrom, L, et al., 1985, Strong antitumor activities of IgG3 antibodies to a human melanoma-associated ganglioside, Proc. Nat'l Acad. Sci. USA 82 (1985) 1499-1502
Hu S. Z. et al., 1996, Minibody: A novel engineered anti-carcinoembryonic antigen antibody fragment (single-chain Fv-CH3) which exhibits rapid, high-level targeting of xenografts, Cancer Research, 56, 3055-3061
Huang C-Y., et al., 2012, Optimizing radioimmunoconjugate delivery in the treatment of solid tumor, Cancer Treat Rev., 38(7): 854-60
Hussain F. et al., 2013, Human anti-macrophage migration inhibitory factor antibodies inhibit growth of human prostate cancer cells in vitro and in vivo, Mol Cancer Ther. Jul; 12(7): 1223-34
Ianello A. and Ahmad A., 2005, Role of antibody-dependent cell-mediated cytotoxicity in the efficacy of therapeutic anti-cancer monoclonal antibodies, Cancer and Metastasis Review, 24, 487-499
Jalkanen M. et al., 1985, Heparan sulfate proteoglycans from mouse mammary epithelial cells: localization on the cell surface with a monoclonal antibody, J. Cell. Biol. 101, 976-985
Jalkanen M. et al. 1987, Cell surface proteoglycan of mouse mammary epithelial cells is shed by cleavage of its matrix-binding ectodomain from its membrane-associated domain, J. Cell. Biol. 105, 3087-3096
Kabat et al., Sequences of Proteins of Immunological Interest, Fifth Edition, NIH Publication 91-3242, Bethesda MD (1991), vols. 1-3
Kerschbaumer R. J., et al. 2012 Neutralization of macrophage migration inhibitory factor (MIF) by fully human antibodies correlates with their specificity for the ß-sheet structure of MIF. J Biol Chem. March 2; 287(10):7446-55. doi: 10.1074/jbc.M111.329664. Epub 2012 Jan. 11. PMID: 22238348; PMCID: PMC3293543.
Krebs et al., “Lewin's Genes XI”, Jones & Bartlett Learning, (2017)
Liu S., 2008, Bifunctional coupling agents for radiolabeling of biomolecules and target-specific delivery of metallic radionuclides. Adv Drug Deliv Rev. Sep; 60(12): 1347-70. doi: 10.1016/j.addr.2008.04.006. Epub 2008 Apr. 23. PMID: 18538888; PMCID: PMC2539110.
Mahalingam D. et al., 2015, ASCO Abstract ID2518
Mahalingam D. et al. 2020, Phase I study of Imalumab (BAX69), a fully human recombinant antioxidized macrophage migration inhibitory factor antibody in advanced solid tumours, Br J Clin Pharmacol. 86(9): 1836-1848
Meares et al. 1984, Conjugation of antibodies with bifunctional chelating agents: isothiocyanate and bromoacetamide reagents, methods of analysis and subsequent addition of metal ions. Anal. Biochem. 142, 68-78.
Morrison, S. L., et al., 1984, Chimeric human antibody molecules: mouse antigen-binding domains with human constant region domains, Proc. Natl. Acad. Sci. 81, 6851-6855
Muda M. et al., 2011, Therapeutic assessment of SEED: a new engineered antibody platform designed to generate mono- and bispecific antibodies, Protein Eng. Des. Sel., 24(5), 447-54
Murphy & Weaver, “Janeway's Immunobiology” (9th Ed., or more recent editions), Taylor & Francis Inc, 2017
Natsume A. et al., 2009, “Improving effector functions of antibodies for cancer treatment: Enhancing ADCC and CDC”, Drug, Design, development and Therapy, 3, 7-16
Petkova, S. B., et al., 2006, Enhanced half-life of genetically engineered human IgG1 antibodies in a humanized FcRn mouse model: potential application in humorally mediated autoimmune disease, Int'l. Immunol. 18(12) 1759-1769
Phaeton R., et al., 2016, Beta emitters rhenium-188 and lutetium-177 are equally effective in radioimmunotherapy of HPV-positive experimental cervical cancer. Cancer Med., Jan; 5(1):9-16. doi: 10.1002/cam4.562.
Ravetch, and Kinet, 1991, Fc receptors, Annu. Rev. Immunol. 9, 457-492
Sambrook et al, “Molecular Cloning: A Laboratory Manual” (4th Ed.), Vols. 1-3, Cold Spring Harbor Laboratory Press (2012).
Schinagl, A., et al., 2018, Role of the Cysteine 81 Residue of Macrophage Migration Inhibitory Factor as a Molecular Redox Switch. Biochemistry, 57(9), 1523-1532. https://doi.org/10.1021/acs.biochem.7b01156
Schinagl. A. et al., 2016, Oxidized macrophage migration inhibitory factor is a potential new tissue marker and drug target in cancer, Oncotarget. November 8; 7(45):73486-73496
Spiess C. et al., 2015, Alternative molecular formats and therapeutic applications for bispecific antibodies, Mol. Immunol., 67, 95-106
Thiele M. et al., (2015), A fully human anti-oxMIF antibody penetrates colorectal metastasis and accumulates in cancer tissue, Annals of Oncology, 26, ii16
Thiele, M. et al., (2015). Selective Targeting of a Disease-Related Conformational Isoform of Macrophage Migration Inhibitory Factor Ameliorates Inflammatory Conditions. Journal of Immunology, 195(5), 2343-2352. https://doi.org/10.4049/jimmunol. 1500572
Van der Kant, R. et al., 2017, Prediction and Reduction of the Aggregation of Monoclonal Antibodies, J. Mol. Biol., 429(8), 1244-1261
Warram J. M. et al, 2014, Antibody-based imaging strategies for cancer, Cancer Metastasis Rev, 33(2-3), 809-22
Wei Li et al., 2016, Antibody Aggregation: Insights from Sequence and Structure, Antibodies, 5(3), 19
Willuda J. et al., 1999, High thermal stability is essential for tumor targeting of antibody fragments: engineering of a humanized anti-epithelial glycoprotein-2 (epithelial cell adhesion molecule) single-chain Fv fragment, Cancer Res., 15; 59(22), 5758-67
Wu G. Y. and Wu C. H., 1987, Receptor-mediated in vitro gene transformation by a soluble DNA carrier system, J. Biol. Chem. 262:4429-4432 (1987)).
Zeglis B. M. and Lewis, J. S. 2011, A practical guide to the construction of radiometallated bioconjugates for positron emission tomography, Dalton trans., 40(23), 6168-95

1. A radioimmunoconjugate comprising a recombinant anti-oxMIF antibody or an antigen binding fragment thereof, comprising:
(a) a light chain variable domain comprising:
(i) SEQ ID NO: 32 having at least one of amino acid substitutions M30L, F49Y, A51G, P80S, or W93F, or
(ii) SEQ ID NO: 32 with 1, 2, 3, 4, or 5 amino acid substitutions, further comprising a conserved tyrosine at position 36, and at least one of amino acid substitutions M30L, F49Y, A51G, P80S, or W93F; and
(b) a heavy chain variable domain comprising:
(i) SEQ ID NO: 29,
(ii) SEQ ID NO: 29 and the amino acid substitutions L5Q and/or W97Y, or
(iii) SEQ ID NO: 29 having at least one of amino acid substitutions L5Q or W97Y, and 1, 2, 3, 4 or 5 further amino acid substitutions,
wherein amino acid positions are numbered according to Kabat, and
wherein said antibody or antigen binding fragment thereof has reduced aggregation potential and reduced hydrophobicity compared to an antibody or an antigen binding fragment thereof comprising SEQ ID NO: 32 and SEQ ID NO: 29 lacking the amino acid substitutions.

2. The radioimmunoconjugate of claim 1, wherein the light chain variable domain comprises the amino acid substitution W93F and the heavy chain variable region comprises the amino acid substitution W97Y.

3. The radioimmunoconjugate of claim 1, wherein the radioisotope is selected from the group consisting of ¹¹C, ¹³N, ¹⁵O, ¹⁸F, ³²p, ⁶⁴Cu, ⁶⁷Cu, ⁶⁷Ga, ⁸⁹Zr, ⁹⁰Y, ⁹⁹mTc, ¹⁰³Pd, ¹⁰⁵Rh, ¹⁰⁹Pd, ¹¹¹ Ag, ¹¹¹In, ¹²³I, ¹²⁴I, ¹²⁵I, ¹³¹I, ¹⁴⁰La, ¹⁴⁹Tb, ¹⁴⁹Pm, ¹⁵³Sm, ¹⁵⁹Gd, ¹⁶⁵Dy, ¹⁶⁶Dy, ¹⁶⁶Ho, ¹⁶⁹Yb, ¹⁷⁵Yb, ¹⁷⁷Lu, ²²⁷Th, ¹⁸⁶Re, ¹⁸⁸Re, ¹⁹²Ir, ¹⁹³mPt, ¹⁹⁵mPt, ¹⁹⁸Au, ¹⁹⁹Au, ²¹¹At, ²¹²Pb, ²¹²Bi, ²¹³Bi, ²²⁵ Ac, ²²³Ra, and ²²⁷Th, specifically the radioisotope is selected from the group consisting of ⁶⁷Ga, ⁸⁹Zr, ¹¹¹In, ¹²⁴I, ¹³¹I, ¹⁷⁷Lu, and ²²⁵Ac.

4. The radioimmunoconjugate of claim 1, comprising a Fc variant domain of a wild-type human IgG1 constant domain having SEQ ID NO: 37 which comprises at least one amino acid substitution at any one of positions, L234, L235, G236, G237, N297, L328 or P329, wherein said radioimmunoconjugate exhibits decreased binding to Fcγ-receptors compared to a radioimmunoconjugate comprising the wildtype IgG1 Fc region.

5. The radioimmunoconjugate of claim 1, comprising an Fc variant domain of a human IgG1 constant domain and comprising one, two or three amino acid substitutions at any one of positions, I253, H310, H435, wherein said radioimmunoconjugate exhibits reduced affinity to the human FcRn compared to a radioimmunoconjugate comprising the wildtype IgG1 Fc region

6. The radioimmunoconjugate of claim 1, comprising an amino acid sequence selected from the group consisting of SEQ ID NOs: 31, 33, 34, 35, and 36.

7. The radioimmunoconjugate of claim 1, comprising SEQ ID NOs: 29 and 34, SEQ ID NOs: 30 and 33, SEQ ID NOs: 30 and 34, SEQ ID NOs: 31 and 34, SEQ ID NOs: 31 and 36, or SEQ ID NOs: 31 and 33, in combination with any one of SEQ ID NOs: 37, 38, 39, or 40.

8. The radioimmunoconjugate of claim 1, wherein the radioimmunoconjugate is a type of molecule selected from the group consisting of IgG, IgG-fusion protein, Fv, scFv, scFv fusion protein, diabody, diabody fusion protein, Fab, Fab-fusion protein, scFab, scFab-fusion protein, Fab′, and F(ab′)2, Fab′-SH, Fcab, Fcab fusion protein, a fusion protein of two or more single chain antibodies, minibody, and small immune protein (SIP).

9. The radioimmunoconjugate of claim 1, further comprising a chelating group selected from the group of DOTA, deferoxamine B (DFO), and DFO*.

10. (canceled)

11. The radioimmunoconjugate of claim 1, wherein the radioimmunoconjugate is combined with a pharmaceutical carrier or adjuvant to form a pharmaceutical composition.

12. (canceled)

13. An isolated nucleic acid encoding the recombinant anti-oxMIF antibody or antigen binding fragment thereof of the radioimmunoconjugate of claim 1.

14. The nucleic acid of claim 13, wherein the nucleic acid is present in an expression vector.

15. The nucleic acid of claim 14, wherein the vector is present in a host cell.

16. A method for producing the radioimmunoconjugate of claim 1, comprising expressing a nucleic acid encoding an anti-oxMIF antibody in a host cell and further labeling said antibody with a radioisotope.

17.-20. (canceled)

21. A kit for production of the radioimmunoconjugate of claim 1, comprising two or more vials, wherein one vial contains a conjugate comprising a chelator linked to an anti-oxMIF antibody and a second vial contains a radioisotope, wherein the radioisotope is 177Lu or 89Zr, and wherein the content of one or several of the vials is lyophilized or in solution.

22. The kit of claim 21, wherein the content of one or several of the vials is lyophilized or in solution.

23. A method for treating cancer comprising the step of administering a therapeutically effective amount of the radioimmunoconjugate of claim 1 to a subject in need thereof.

24. The method of claim 23, wherein the cancer is selected from the group consisting of colorectal cancer, ovarian cancer, breast cancer, prostate cancer, pancreas cancer, and lung cancer.

25. The method of claim 23, wherein a hematological tumor or solid tumor in the subject is treated.

26. The method of claim 23, further comprising the step of detecting tumor cells in a biological sample from the subject, thereby determining that the subject has cancer.

27. The radioimmunoconjugate of claim 11, wherein the pharmaceutical composition is formulated for intravenous administration.
